(19) United States
(12) Patent Application Publication
(10) Pub. No.: US 2026/0144883 A1
(43) Pub. Date: May 28, 2026

(54) ANTI-TROP2/EGFR ANTIBODIES AND USES THEREOF

(71) Applicant: XADCERA BIOPHARMACEUTICAL (SUZHOU) CO., LTD., Jiangsu (CN)

(72) Inventors: Zhuolin LI, Beijing (CN); Xuewa GUAN, Suzhou (CN); Chengzhang SHANG, Beijing (CN); Yuelei SHEN, Beijing (CN)

(21) Appl. No.: 19/070,990

(22) Filed: Mar. 5, 2025

Related U.S. Application Data
(63) Continuation-in-part of application No. PCT/CN2023/117376, filed on Sep. 7, 2023.

(30) Foreign Application Priority Data
Sep. 7, 2022 (WO) PCT/CN2022/117496
Mar. 23, 2023 (WO) PCT/CN2023/083228

(51) Int. Cl. A61K 47/68; A61K 45/06; A61P 35/00; C07K 16/28; C07K 16/30

(52) U.S. Cl. CPC A61K 47/68037; A61K 45/06; A61K 47/68031; A61K 47/6879; A61K 47/6889; A61P 35/00; C07K 16/2818; C07K 16/2863; C07K 16/30; C07K 2317/24; C07K 2317/31; C07K 2317/33; C07K 2317/565; C07K 2317/622; C07K 2317/92; C07K 2317/94

(57) ABSTRACT

This disclosure relates to anti-TROP2/EGFR antibodies, and antibody drug conjugates derived therefrom, wherein the antibodies specifically bind to at least two different antigens EGFR and TROP2.

### CROSS REFERENCE TO RELATED APPLICATIONS

This application is a continuation-in-part of International Application No. PCT/CN2023/117376 filed Sep. 7, 2023, which claims priority to PCT/CN2022/117496 filed on Sep. 7, 2022, and PCT/CN2023/083228, filed on Mar. 23, 2023. The entire contents of each of the foregoing applications is incorporated herein by reference.

### SEQUENCE LISTING

This application contains references to amino acid sequences and/or nucleic acid sequences which have been submitted concurrently herewith as the sequence listing file entitled “UNIT.P0028US.xml”, file size 35,973 bytes, created on Mar. 5, 2025. The aforementioned sequence listing is hereby incorporated by reference in its entirety.

### TECHNICAL FIELD

This disclosure relates to multi-specific anti-TROP2/EGFR antibodies (e.g., bispecific antibodies or antigen-binding fragments thereof), and antibody drug conjugates derived therefrom.

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications, there is a need to continue to develop various therapeutics based on bispecific antibodies.

### SUMMARY

This disclosure relates to anti-TROP2/EGFR antibodies or antigen-binding fragments thereof, wherein the antibodies or antigen-binding fragments thereof specifically bind to EGFR and TROP2. In some embodiments, the antibodies or antigen-binding fragments thereof have identical light chain variable regions. In some embodiments, the antibodies or antigen-binding fragments thereof have a common light chain. The disclosure also relates to antibody drug conjugates derived from these anti-TROP2/EGFR antibodies.

In one aspect, the disclosure provides an anti-TROP2/EGFR antibody or antigen-binding fragment thereof, comprising: a first antigen-binding domain that specifically binds to EGFR; and a second antigen-binding domain that specifically binds to TROP2.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2). In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively; and
(4) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively; and
(2) the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 4-6, respectively, and the selected VL2 CDRs 1, 2, 3 amino acid sequences are set forth in SEQ ID NOs: 1-3, respectively.

In some embodiments, the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 13-15, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95%, 99%, or 100% identical to SEQ ID NO: 23, the first light chain variable region comprises a sequence that is at least 80%, 85%, 90%, 95%, 99%, or 100% identical to SEQ ID NO: 22, the second heavy chain variable region comprises a sequence that is at least 80%, 85%, 90%, 95%, 99%, or 100% identical to SEQ ID NO: 25, and the second light chain variable region comprises a sequence that is at least 80%, 85%, 90%, 95%, 99%, or 100% identical to SEQ ID NO: 22.

In some embodiments, the first heavy chain variable region comprises a sequence that is at least 80%, 85%, 90%, 95%, 99%, or 100% identical to SEQ ID NO: 24, the first light chain variable region comprises a sequence that is at least 80%, 85%, 90%, 95%, 99%, or 100% identical to SEQ ID NO: 22, the second heavy chain variable region comprises a sequence that is at least 80%, 85%, 90%, 95%, 99%, or 100% identical to SEQ ID NO: 25, and the second light chain variable region comprises a sequence that is at least 80%, 85%, 90%, 95%, 99%, or 100% identical to SEQ ID NO: 22.

In some embodiments, the VH1 comprises an amino acid sequence that is at least 90% identical to a selected VH sequence, and the VL1 comprises an amino acid sequence that is at least 90% identical to a selected VL sequence, wherein the selected VH sequence and the selected VL sequence are one of the following:
(1) the selected VH sequence is SEQ ID NO: 23, and the selected VL sequence is SEQ ID NO: 22; and
(2) the selected VH sequence is SEQ ID NO: 24, and the selected VL sequence is SEQ ID NO: 22.

In some embodiments, the VH1 comprises VH CDR1, VH CDR2, and VH CDR3 that are identical to VH CDR1, VH CDR2, and VH CDR3 of a selected VH sequence; and the VL1 comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 23, and the selected VL sequence is SEQ ID NO: 22; and
(2) the selected VH sequence is SEQ ID NO: 24, and the selected VL sequence is SEQ ID NO: 22.

In some embodiments, the VH2 comprises an amino acid sequence that is at least 90% identical to a selected VH sequence, and the VL2 comprises an amino acid sequence that is at least 90% identical to a selected VL sequence, wherein the selected VH sequence is SEQ ID NO: 25, and the selected VL sequence is SEQ ID NO: 22.

In some embodiments, the VH2 comprises VH CDR1, VH CDR2, and VH CDR3 that are identical to VH CDR1, VH CDR2, and VH CDR3 of a selected VH sequence; and the VL2 comprising VL CDR1, VL CDR2, and VL CDR3 that are identical to VL CDR1, VL CDR2, and VL CDR3 of a selected VL sequence, wherein the selected VH sequence is SEQ ID NO: 25, and the selected VL sequence is SEQ ID NO: 22.

In some embodiments, the VH1 comprises the sequence of SEQ ID NO: 23 and the VL1 comprises the sequence of SEQ ID NO: 22.

In some embodiments, the VH1 comprises the sequence of SEQ ID NO: 24 and the VL1 comprises the sequence of SEQ ID NO: 22.

In some embodiments, the VH2 comprises the sequence of SEQ ID NO: 25 and the VL2 comprises the sequence of SEQ ID NO: 22.

In some embodiments, the first antigen-binding domain specifically binds to human or monkey EGFR; and/or the second antigen-binding domain specifically binds to human or monkey TROP2.

In some embodiments, the first antigen-binding domain is human or humanized; and/or the second antigen-binding domain is human or humanized.

In some embodiments, the anti-TROP2/EGFR antibody is a multi-specific antibody (e.g., a bispecific antibody).

In some embodiments, the first antigen-binding domain is a single-chain variable fragment (scFv); and/or the second antigen-binding domain is a scFv.

In some embodiments, the first light chain variable region and the second light chain variable region are identical.

In one aspect, the disclosure provides an anti-TROP2/EGFR antibody or antigen-binding fragment thereof that cross-competes with the anti-TROP2/EGFR antibody or antigen-binding fragment thereof as described herein.

In one aspect, the disclosure provides a nucleic acid comprising a polynucleotide encoding the anti-TROP2/EGFR antibody or antigen-binding fragment thereof as described herein.

In one aspect, the disclosure provides a vector comprising the nucleic acid as described herein.

In one aspect, the disclosure provides a cell comprising the vector as described herein. In some embodiments, the cell is a CHO cell.

In one aspect, the disclosure provides a cell comprising the nucleic acid as described herein.

In one aspect, the disclosure provides a method of producing an anti-TROP2/EGFR antibody or an antigen-binding fragment thereof, the method comprising
(a) culturing the cell as described herein under conditions sufficient for the cell to produce the anti-TROP2/EGFR antibody or the antigen-binding fragment thereof; and
(b) collecting the anti-TROP2/EGFR antibody or the antigen-binding fragment thereof produced by the cell.

In one aspect, the disclosure provides an anti-TROP2/EGFR antibody-drug conjugate (ADC) comprising a therapeutic agent covalently bound to the anti-TROP2/EGFR antibody or antigen-binding fragment thereof as described herein. In some embodiments, the therapeutic agent is a cytotoxic or cytostatic agent. In some embodiments, the therapeutic agent is MMAE or MMAF.

In some embodiments, the therapeutic agent is selected from

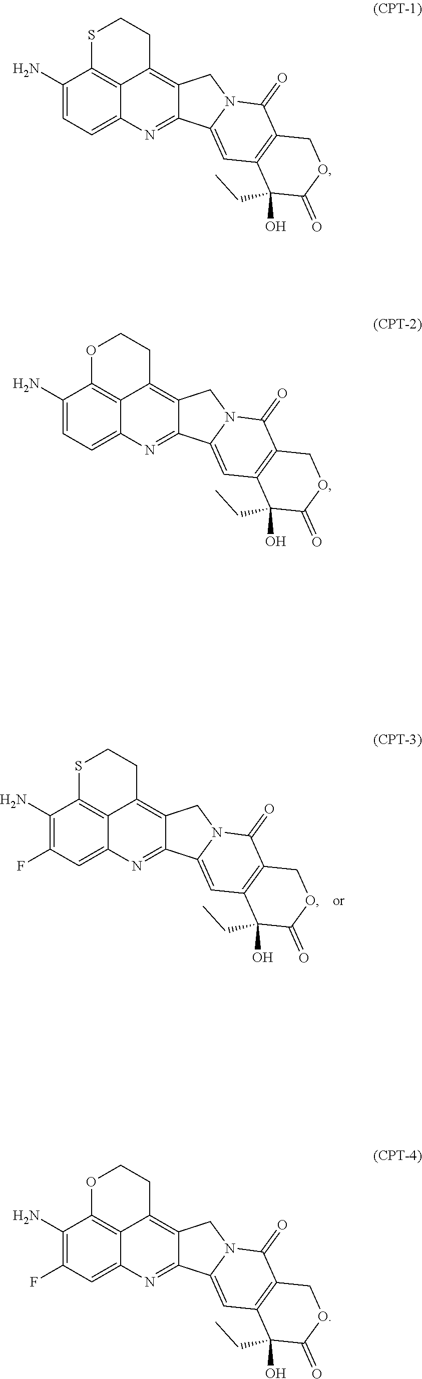 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

In some embodiments, the therapeutic agent is linked to the antibody or antigen-binding fragment thereof via a linker. In some embodiments, the linker has a structure of:

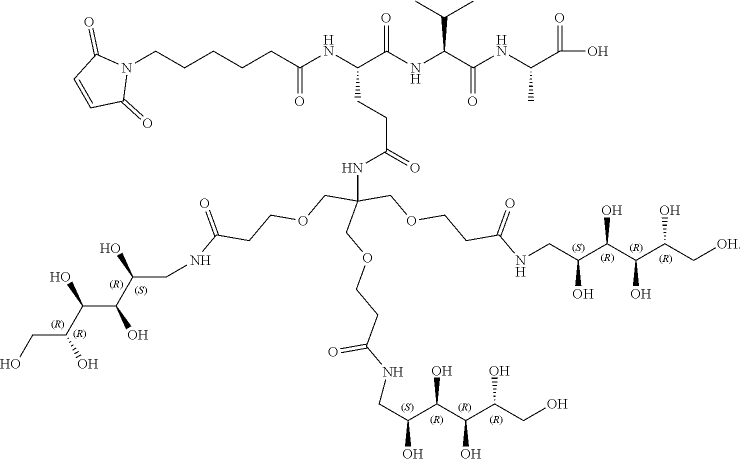

In some embodiments, the antibody-drug conjugate has a structure of:

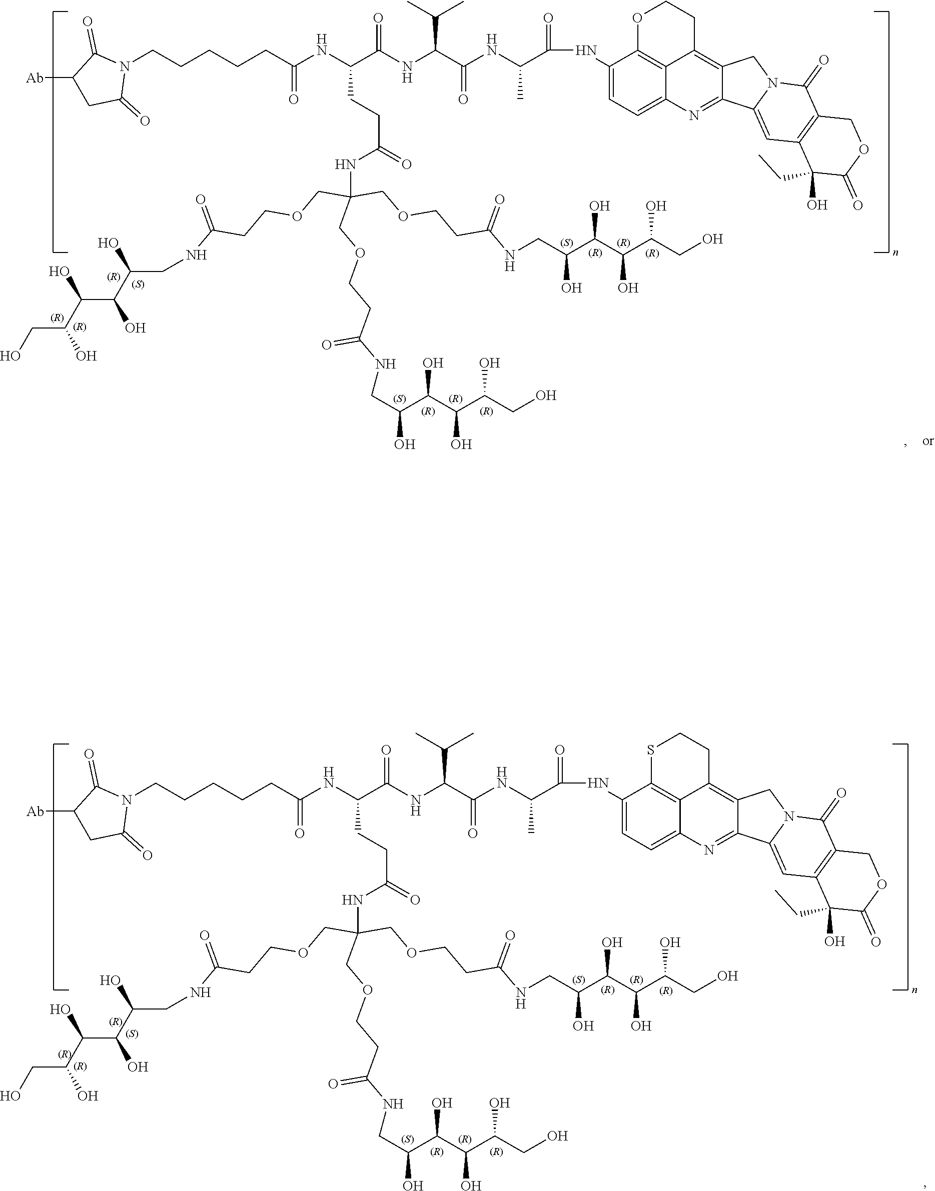

in some embodiments, n=1-8; in some embodiments, “Ab” represents the antibody or antigen-binding fragment thereof.

In some embodiments, the drug-to-antibody ratio (DAR) is about 4 or 8.

In one aspect, the disclosure provides a method of treating a subject having cancer, the method comprising administering a therapeutically effective amount of a composition comprising the anti-TROP2/EGFR antibody or antigen-binding fragment thereof as described herein, or the anti-TROP2/EGFR antibody-drug conjugate as described herein, to the subject. In some embodiments, the subject has a cancer expressing EGFR and/or TROP2.

In some embodiments, the cancer is a solid tumor, lung cancer (e.g., non-small cell lung cancer, lung adenocarcinoma, or lung carcinoma), gastric cancer (e.g., gastric carcinoma), skin cancer (e.g., skin carcinoma), colorectal cancer, breast cancer, head and neck cancer, ovarian cancer, prostate cancer, thyroid cancer, pancreatic cancer, CNS cancer, liver cancer, nasopharynx cancer, brain cancer, colon cancer, bladder cancer, oral squamous cell carcinoma, cervical cancer, or oesophageal cancer.

In some embodiments, the subject is a human.

In some embodiments, the method further comprises administering an anti-PD1 antibody to the subject.

In some embodiments, the method further comprises administering a chemotherapy to the subject.

In one aspect, the disclosure provides a method of decreasing the rate of tumor growth, the method comprising contacting a tumor cell with an effective amount of a composition comprising the anti-TROP2/EGFR antibody or antigen-binding fragment thereof one as described herein, or the anti-TROP2/EGFR antibody-drug conjugate one as described herein.

In one aspect, the disclosure provides a method of killing a tumor cell, the method comprising contacting a tumor cell with an effective amount of a composition comprising the anti-TROP2/EGFR antibody or antigen-binding fragment thereof as described herein, or the anti-TROP2/EGFR antibody-drug conjugate as described herein.

In one aspect, the disclosure provides a pharmaceutical composition comprising a pharmaceutically acceptable carrier and (a) the anti-TROP2/EGFR antibody or antigen-binding fragment thereof as described herein, and/or (b) the anti-TROP2/EGFR antibody-drug conjugate as described herein.

In one aspect, the disclosure provides an anti-TROP2/EGFR antibody-drug conjugate (ADC) comprising a therapeutic agent covalently bound to a bispecific antibody or antigen-binding fragment thereof comprising: a first antigen-binding domain that specifically binds to EGFR; and a second antigen-binding domain that specifically binds to TROP2. In some embodiments, the drug-to-antibody ratio (DAR) is about 4.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or control antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a control antigen). In other examples, an antigen-binding domain can bind to two different antigens (e.g., an effector antigen and a control antigen).

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, bispecific antibodies, and multi-specific antibodies. One example of an antibody is a protein complex that includes two heavy chains and two light chains. Additional examples of an antibody are described herein.

As used herein, the term “multispecific antibody” is an antibody that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multi-specific antibody binds two different epitopes (i.e., a “bispecific antibody”). In some aspects, a multi-specific antibody binds three different epitopes (i.e., a “trispecific antibody”). In some aspects, a multi-specific antibody binds four different epitopes (i.e., a “quadspecific antibody”). In some aspects, a multi-specific antibody binds five different epitopes (i.e., a “quintspecific antibody”). Each binding specificity may be present in any suitable valency. Non-limiting examples of multi-specific antibodies are described herein.

As used herein, the term “bispecific antibody” refers to an antibody that binds to two different epitopes. The epitopes can be on the same antigen or on different antigens.

As used herein, the term “common light chain” refers to a light chain that can interact with two or more different heavy chains, forming different antigen-binding sites, wherein these different antigen-binding sites can specifically bind to different antigens or epitopes. Similarly, the term “common light chain variable region” refers to a light chain variable region that can interact with two or more different heavy chain variable regions, forming different antigen-binding sites, wherein these different antigen-binding sites can specifically bind to different antigens or epitopes. In some embodiments, the antibody or antigen-binding fragment thereof can have a common light chain. In some embodiments, the anti-TROP2/EGFR antibody or antigen-binding fragment thereof can have a common light chain variable region.

As used herein, the term “anti-TROP2/EGFR antibody or antigen-binding fragment thereof” refers to an antibody or antigen-binding fragment that binds to both TROP2 and EGFR.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing a bispecific anti-TROP2/EGFR antibody having the knobs-into-holes structure with common light chain.

FIG. 2 shows the average tumor volume in different groups of B-NDG mice that were injected with A431 cells, and were treated with phosphate buffer saline (PBS), ADCs or antibodies.

FIG. 3 shows the average tumor volume in different groups of B-NDG mice that were injected with Panc 02.03 cells, and were treated with PBS or ADCs.

FIG. 4 lists heavy chain variable region CDR sequences of anti-EGFR antigen binding domain (E-1G11 and E-6C4) and an anti-TROP2 antigen binding domain (T-6F7) in anti-TROP2/EGFR antibodies as defined by Kabat numbering.

FIG. 5 lists heavy chain variable region CDR sequences of anti-EGFR antigen binding domain (E-1G11 and E-6C4) and an anti-TROP2 antigen binding domain (T-6F7) in anti-TROP2/EGFR antibodies as defined by as defined by Chothia numbering.

FIG. 6 lists CDR sequences for the common light chain as defined by Kabat and Chothia numbering.

FIG. 7 lists anti-TROP2/EGFR antibody heavy chain and light chain variable region sequences discussed in the disclosure.

FIG. 8 lists additional amino acid sequences discussed in the disclosure.

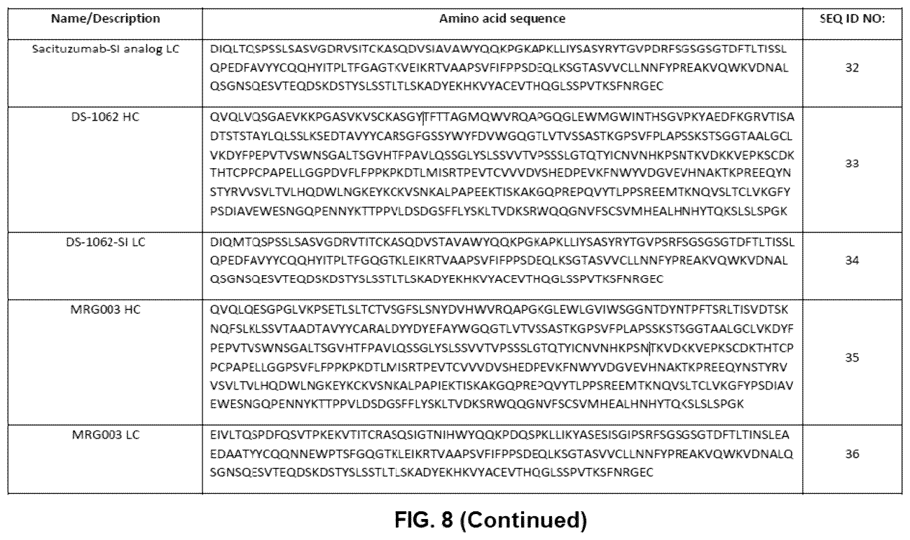

FIG. 9A shows the killing efficacy of T-6F7-E-6C4-ADC against BxPC-3 cells+NCI-H520 cells, BxPC-3 cells or NCI-H520 cells.

FIG. 9B shows the killing efficacy of T-6F7-E-6C4-ADC (0.1 μg/mL) against BxPC-3 cells+NCI-H520 cells after co-incubating for 72 hours.

FIG. 10 shows the average tumor volume in different groups of B-NDG mice that were injected with NCI-H292 cells, and were treated with PBS, antibodies, or ADCs.

FIG. 11 shows the average tumor volume in different groups of B-NDG mice that were injected with NUGC-4 cells, and were treated with PBS, antibodies, or ADCs.

FIGS. 12A-12B show the serum concentration of ADC and total antibody after administration of ISO-ADC (FIG. 12A) or T-6F7-E-6C4-ADC (FIG. 12B) to B-hFcRn mice.

FIG. 13 shows the ratios of free MMAE to ADC in plasma of human, Macaca fascicularis, or SD rat in 0 day, 1 day, 2 days, 6 days, 8 days, 11 days and 14 days after the adding of T-6F7-E-6C4-ADC to the plasma.

FIGS. 14A-14B show endocytosis activities of anti-TROP2/EGFR bispecific antibody and ADCs in A431 cells (FIG. 14A) or NCI-H292 cells (FIG. 14B). ISO-CPT2(DAR8) was used as an isotype control. Sacituzumab govitecan and Cetuximab were used as controls.

FIG. 15 shows the average tumor volume in different groups of B-NDG mice that were injected with patient-derived breast tumor fragments, and were treated with PBS or ADCs.

FIG. 16 shows the average tumor volume in different groups of B-NDG mice that were injected with SKOV-3 cells, and were treated with PBS or ADCs.

FIG. 17 shows the average tumor volume in different groups of B-NDG mice that were injected with A431 cells, and were treated with PBS, antibody, or ADCs.

FIG. 18A shows the average tumor volume in different groups of B-NDG mice that were injected with head and neck squamous cell carcinoma patient-derived tumor fragments, and were treated with T-6F7-E-6C4-CPT2(DAR8). Saline was used as a control.

FIG. 18B shows the average tumor volume in different groups of B-NDG mice that were injected with esophageal cancer patient-derived tumor fragments, and were treated with T-6F7-E-6C4-CPT2(DAR8). Saline was used as a control.

FIG. 18C and FIG. 18D show the average tumor volume in different groups of B-NDG mice that were injected with colorectal cancer patient-derived tumor fragments, and were treated with T-6F7-E-6C4-CPT2(DAR8). Saline was used as a control.

FIG. 18E and FIG. 18F show the average tumor volume in different groups of B-NDG mice that were injected with gastric cancer patient-derived tumor fragments, and were treated with T-6F7-E-6C4-CPT2(DAR8). Saline was used as a control.

FIGS. 19A-19B show the serum concentration of total antibody (FIG. 19A) and CPT2 (FIG. 19B) after administration of T-6F7-E-6C4-CPT2(DAR4) or T-6F7-E-6C4-CPT2(DAR8) to B-NDG mice.

FIGS. 19C-19D show the tumor tissue concentration of total antibody (FIG. 19C) and CPT2 (FIG. 19D) after administration of T-6F7-E-6C4-CPT2(DAR4) or T-6F7-E-6C4-CPT2(DAR8) to B-NDG mice.

FIGS. 20A-20B show the ratios of free CPT2 to total ADC in plasma of human, Macaca fascicularis, or SD rat in 0 day, 1 day, 2 days, 6 days, 8 days, 11 days and 14 days after the adding of T-6F7-E-6C4-CPT2(DAR4) (FIG. 20A) or T-6F7-E-6C4-CPT2(DAR8) (FIG. 20B) to the plasma.

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms. Each arm can have one heavy chain variable region and one light chain variable region, forming an antigen-binding domain (or an antigen-binding region). In some embodiments, the bispecific antibody has a common light chain.

The present disclosure relates to anti-TROP2/EGFR antibodies (e.g., bispecific antibodies or antigen-binding fragments thereof) that specifically bind to EGFR and TROP2, and antibody drug conjugates derived from these anti-TROP2/EGFR antibodies.

### Anti-TROP2/EGFR Antibody

Epidermal growth factor receptor (EGFR, ErbBI or HER1) is a Type 1 transmembrane glycoprotein of 170 kDa that is encoded by the c-erbB1 proto-oncogene. The epidermal growth factor receptor is a member of the ErbB family of receptors, a subfamily of four closely related receptor tyrosine kinases: EGFR (ErbB-1), HER2/neu (ErbB-2), Her3 (ErbB-3) and Her4 (ErbB-4). In many cancer types, mutations affecting EGFR expression or activity could result in cancer. EGFR signaling is initiated by ligand binding followed by induction of conformational change, homodimerization or heterodimerization of the receptor with other ErbB family members, and trans-autophosphorylation of the receptor, which initiates signal transduction cascades that ultimately affect a wide variety of cellular functions, including cell proliferation and survival, increases in expression or kinase activity of EGFR have been linked with a range of human cancers, making EGFR an attractive target for therapeutic intervention. Increases in both the EGFR gene copy number and protein expression have been associated with favorable responses to the EGFR tyrosine kinase inhibitor, IRESSA™ (gefitinib), in non-small cell lung cancer.

Binding of a ligand such as EGF (epidermal growth factor) to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Inhibition of EGFR signaling may result in inhibition in one or more EGFR. In some embodiments, the EGFR ligands include EGF, TGFα, heparin binding EGF (HB-EGF), amphiregulin (AR), and epiregulm (EPI).

A detailed review of EGFR can be found in Sabbah, Dima A., Rima Hajjo, and Kamal Sweidan. “Review on epidermal growth factor receptor (EGFR) structure, signaling pathways, interactions, and recent updates of EGFR inhibitors.” Current topics in medicinal chemistry (2020); which is incorporated herein by reference in its entirety.

Trophoblast cell-surface antigen 2 (TROP2), also known as Tumor-associated calcium signal transducer 2 (TACSTD2), is a cell surface glycoprotein encoded and expressed by the TACSTD2 gene. It has high structural sequence similarity with epithelial adhesion molecule Epcam. TROP2 is a protein closely related to tumors. It mainly promotes tumor cell growth, proliferation and metastasis by regulating calcium ion signaling pathways, cyclin expression, and reducing fibronectin adhesion. Studies have found that TROP2 protein is highly expressed in breast cancer, colon cancer, bladder cancer, gastric cancer, oral squamous cell carcinoma and ovarian cancer. The protein can promote tumor cell proliferation, invasion, metastasis, spread and other processes. In addition, in breast cancer and other cancers, the high expression of TROP2 has also been found to be closely related to more aggressive diseases and poor clinical prognosis of tumors.

TROP2 is an intracellular calcium signal transducer that is differentially expressed in many cancers. It signals cells for self-renewal, proliferation, invasion, and survival. It has stem cell-like qualities. TROP2 is expressed in many normal tissues, though in contrast, it is overexpressed in many cancers and the overexpression of TROP2 is of prognostic significance. Several ligands have been proposed that interact with TROP2. TROP2 signals the cells via different pathways and it is transcriptionally regulated by a complex network of several transcription factors. TROP2 expression in cancer cells has been correlated with drug resistance.

A detailed review of TROP2 and its overexpression in cancers can be found in Shvartsur, Anna, and Benjamin Bonavida. “TROP2 and its overexpression in cancers: regulation and clinical/therapeutic implications.” Genes & cancer 6.3-4 (2015): 84; which is incorporated herein by reference in its entirety.

In some embodiments, the bispecific anti-TROP2/EGFR antibody described herein can be designed to have an IgG1 subtype structure with knobs-into-holes (KIH) mutations, which can promote heterodimerization and avoid mispairing between the two heavy chains. In some embodiments, the bispecific anti-TROP2/EGFR antibody has a higher endocytosis rate than the corresponding monoclonal antibodies or the control bispecific antibodies.

In some embodiments, the bispecific anti-TROP2/EGFR antibody described herein can be conjugated with a therapeutic agent, forming an antibody drug conjugate (ADC). In some embodiments, the drug-to-antibody ratio (DAR) of the ADCs described herein is about 3.8, about 3.9, about 4.0, about 4.1, about 4.2, about 4.3, about 4.4, about 4.5, about 4.6, or about 4.7. In some embodiments, the DAR of the ADCs described herein is about 3.5 to about 4.5, about 3.6 about 4.5, about 3.7 to about 4.5, about 3.8 to about 4.5, about 3.9 to about 4.5, about 4.0 to about 4.5, about 4.1 to about 4.5, about 4.2 to about 4.5, about 4.3 to about 4.5, about 4.4 to about 4.5, about 3.5 to about 4.4, about 3.6 to about 4.4, about 3.7 to about 4.4, about 3.8 to about 4.4, about 3.9 to about 4.4, about 4.0 to about 4.4, about 4.1 to about 4.4, about 4.2 to about 4.4, about 4.3 to about 4.4, about 3.5 to about 4.3, about 3.6 to about 4.3, about 3.7 to about 4.3, about 3.8 to about 4.3, about 3.9 to about 4.3, about 4.0 to about 4.3, about 4.1 to about 4.3, about 4.2 to about 4.3, about 3.5 to about 4.2, about 3.6 to about 4.2, about 3.7 to about 4.2, about 3.8 to about 4.2, about 3.9 to about 4.2, about 4.0 to about 4.2, about 4.1 to about 4.2, about 3.5 to about 4.1, about 3.6 to about 4.1, about 3.7 to about 4.1, about 3.8 to about 4.1, about 3.9 to about 4.1, about 4.0 to about 4.1, about 3.5 to about 4.0, about 3.6 to about 4.0, about 3.7 to about 4.0, about 3.8 to about 4.0, about 3.9 to about 4.0, about 3.5 to about 3.9, about 3.6 to about 3.9, about 3.7 to about 3.9, about 3.8 to about 3.9, about 3.5 to about 3.8, about 3.6 to about 3.8, about 3.7 to about 3.8, about 3.5 to about 3.7, about 3.6 to about 3.7, or about 3.5 to about 3.6. In some embodiments, the DAR of the ADCs described herein is about 7.5 to about 8.5, about 7.6 to about 8.5, about 7.7 to about 8.5, about 7.8 to about 8.5, about 7.9 to about 8.5, about 8.0 to about 8.5, about 8.1 to about 8.5, about 8.2 to about 8.5, about 8.3 to about 8.5, about 8.4 to about 8.5, about 7.5 to about 8.4, about 7.6 to about 8.4, about 7.7 to about 8.4, about 7.8 to about 8.4, about 7.9 to about 8.4, about 8.0 to about 8.4, about 8.1 to about 8.4, about 8.2 to about 8.4, about 8.3 to about 8.4, about 7.5 to about 8.3, about 7.6 to about 8.3, about 7.7 to about 8.3, about 7.8 to about 8.3, about 7.9 to about 8.3, about 8.0 to about 8.3, about 8.1 to about 8.3, about 8.2 to about 8.3, about 7.5 to about 8.2, about 7.6 to about 8.2, about 7.7 to about 8.2, about 7.8 to about 8.2, about 7.9 to about 8.2, about 8.0 to about 8.2, about 8.1 to about 8.2, about 7.5 to about 8.1, about 7.6 to about 8.1, about 7.7 to about 8.1, about 7.8 to about 8.1, about 7.9 to about 8.1, about 8.0 to about 8.1, about 7.5 to about 8.0, about 7.6 to about 8.0, about 7.7 to about 8.0, about 7.8 to about 8.0, about 7.9 to about 8.0, about 7.5 to about 7.9, about 7.6 to about 7.9, about 7.7 to about 7.9, about 7.8 to about 7.9, about 7.5 to about 7.8, about 7.6 to about 7.8, about 7.7 to about 7.8, about 7.5 to about 7.7, about 7.6 to about 7.7, or about 7.5 to about 7.6.

In some embodiments, the anti-TROP2/EGFR ADC described herein can effectively inhibit in vitro cancer cell growth at a concentration of less than 10 μg/mL, less than 3.33 μg/mL, less than 1.11 μg/mL, less than 0.37 μg/mL, less than 0.12 μg/mL, less than 0.04 μg/mL, or less than 0.01 μg/mL. In some embodiments, the anti-TROP2/EGFR ADC described herein can inhibit in vivo cancer cell growth (e.g., lung cancer, gastric cancer, or skin cancer) in a xenograft mouse model at a dose level of less than 30 mg/kg, 25 mg/kg, 20 mg/kg, 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

In some embodiments, the anti-TROP2/EGFR antibody described herein has a common light chain. In some embodiments, the anti-TROP2/EGFR antibody includes an anti-EGFR antigen-binding domain (e.g., E-1G11 (“1G11”), E-6C4 (“6C4”)) or an anti-TROP2 antigen-binding domain (e.g., T-6F7 (“6F7”)). In some embodiments, the anti-TROP2/EGFR antibodies have a heavy chain variable region targeting EGFR (e.g., any one of the VH targeting EGFR described herein), a heavy chain variable region targeting TROP2 (e.g., any one of the VH targeting TROP2 described herein), and two identical common light chain variable regions.

The CDR sequences for 1G11 antigen-binding domain include CDRs of the heavy chain variable domain, SEQ ID NOs: 7-9, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 16-18, and CDR sequences of the light chain variable domain are set forth in SEQ ID NOs: 1-3. The human light chain variable region and human heavy chain variable region for 1G11 are shown in SEQ ID NO: 22 and SEQ ID NO: 23, respectively.

The CDR sequences for 6C4 antigen-binding domain include CDRs of the heavy chain variable domain, SEQ ID NOs: 10-12, and CDRs of the light chain variable domain, SEQ ID NOs: 1-3, as defined by Kabat numbering. Under Chothia numbering, the CDR sequences of the heavy chain variable domain are set forth in SEQ ID NOs: 19-21, and CDRs of the light chain variable domain are set forth in SEQ ID NOs: 1-3. The human light chain variable region and human heavy chain variable region for 6C4 are shown in SEQ ID NO: 22 and SEQ ID NO: 24, respectively.

In some embodiments, the anti-TROP2/EGFR antibodies described herein can contain one, two, or three heavy chain variable region CDRs selected from the group of SEQ ID NOs: 7-9, SEQ ID NOs: 10-12, SEQ ID NOs: 16-18, and SEQ ID NOs: 19-21; and/or one, two, or three light chain variable region CDRs selected from the group of SEQ ID NOs: 1-3.

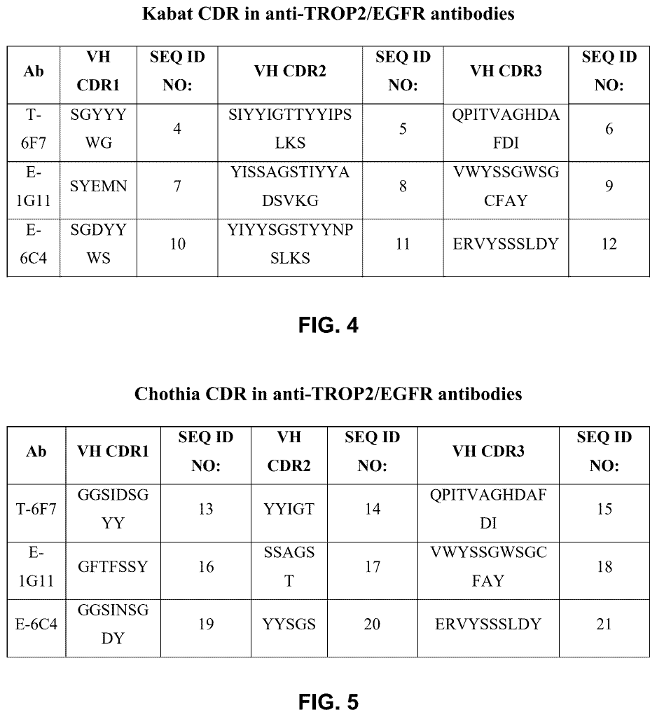

In some embodiments, the anti-TROP2/EGFR antibodies or antigen-binding fragments thereof can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 4-6.

In some embodiments, the anti-TROP2/EGFR antibody or antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the anti-TROP2/EGFR antibody or antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the anti-TROP2/EGFR antibody or antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the anti-TROP2/EGFR antibody or antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the anti-TROP2/EGFR antibody or antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence.

In some embodiments, the anti-TROP2/EGFR antibodies contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 23 or 24, and the selected VL sequence is SEQ ID NO: 22.

The CDR sequences for 6F7 antigen-binding domain include CDRs of the heavy chain variable domain, SEQ ID NOs: 4-6, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 13-15, and CDR sequences of the light chain variable domain are set forth in SEQ ID NOs: 1-3. The human light chain variable region and human heavy chain variable region for 6F7 are shown in SEQ ID NO: 22 and SEQ ID NO: 25, respectively.

Furthermore, in some embodiments, the anti-TROP2/EGFR antibodies or antigen-binding fragments thereof described herein can also contain one, two, or three heavy chain variable region CDRs selected from the group of SEQ ID NOs: 4-6 and SEQ ID NOs: 13-15; and/or one, two, or three light chain variable region CDRs selected from the group of SEQ ID NOs: 1-3.

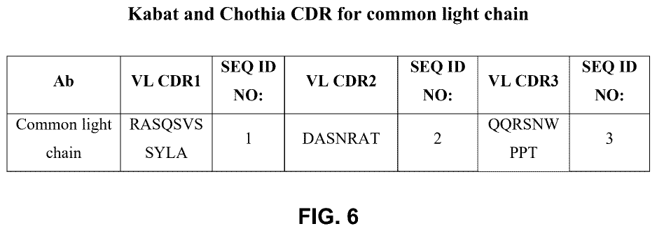

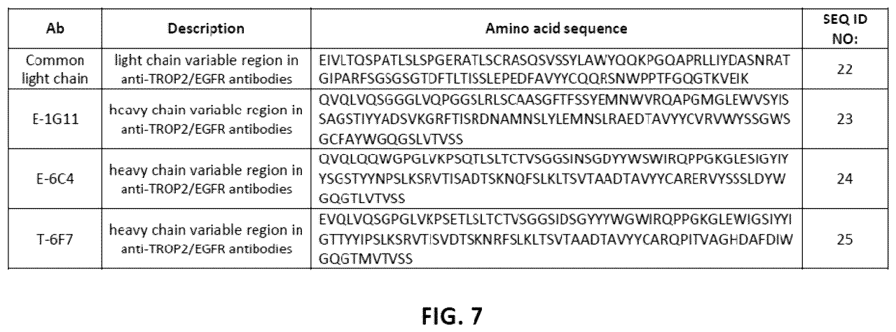

In some embodiments, the anti-TROP2/EGFR antibodies or antigen-binding fragments thereof can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 4 and 6 (Kabat CDR) and FIGS. 5 and 6 (Chothia CDR).

In some embodiments, the anti-TROP2/EGFR antibody or antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the anti-TROP2/EGFR antibody or antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the anti-TROP2/EGFR antibody or antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence.

In some embodiments, the anti-TROP2/EGFR antibodies contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 25, and the selected VL sequence is SEQ ID NO: 22.

In some embodiments, the anti-TROP2/EGFR antibody or antigen-binding fragment can have 3 VH CDRs that are identical to the CDRs of any VH sequences as described herein. In some embodiments, the anti-TROP2/EGFR antibody or antigen-binding fragment can have 3 VL CDRs that are identical to the CDRs of any VL sequences as described herein.

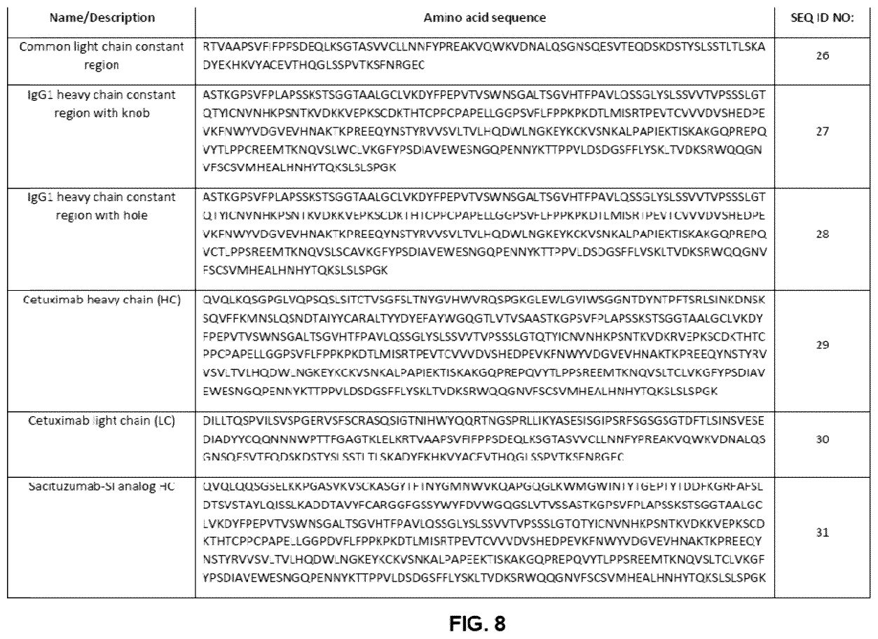

The disclosure also provides nucleic acid comprising a polynucleotide encoding an anti-TROP2/EGFR antibody. The immunoglobulin heavy chain or immunoglobulin light chain in the anti-TROP2/EGFR antibody comprises CDRs as shown in FIG. 4, FIG. 5, or FIG. 6, or have sequences as shown in FIG. 7. When the polypeptides are paired with corresponding polypeptide (e.g., a corresponding heavy chain variable region or a corresponding light chain variable region), the paired polypeptides bind to TROP2 and/or EGFR.

The anti-TROP2/EGFR antibodies can also be anti-TROP2/EGFR antibody variants (including derivatives and conjugates) of anti-TROP2/EGFR antibodies or antibody fragments. Additional anti-TROP2/EGFR antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-TROP2/EGFR antibodies can be of any type (e.g., IgG, IgE, IgM, IgD, IgA, and IgY), class (e.g., IgG1, IgG2, IgG3, IgG4, IgAQ1, and IgA2), or subclass. In some embodiments, the anti-TROP2/EGFR antibody or antigen-binding fragment is an IgG (e.g., IgG1) antibody or antigen-binding fragment thereof.

Fragments of anti-TROP2/EGFR antibodies are suitable for use in the methods provided so long as they retain the desired affinity and specificity to both TROP2 and EGFR. Thus, a fragment of an anti-TROP2/EGFR antibody will retain an ability to bind to TROP2 and EGFR.

### Antibodies and Antigen Binding Fragments Thereof

In some embodiments, the multi-specific anti-TROP2/EGFR antibody (e.g., bispecific antibody) includes an antigen-binding domain that is derived from an anti-EGFR antibody, and an antigen-binding domain that is derived from an anti-TROP2 antibody. These anti-TROP2/EGFR antibodies and antigen-binding fragments thereof can have various forms.

In general, antibodies (also called immunoglobulins) can be made up of two classes of polypeptide chains, light chains and heavy chains. A non-limiting anti-TROP2/EGFR antibody of the present disclosure can be an intact, four immunoglobulin chain antibody comprising two heavy chains and two light chains. The heavy chain of the anti-TROP2/EGFR antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain.

The hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding domain.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen-binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the anti-TROP2/EGFR antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The anti-TROP2/EGFR antibody can also be an immunoglobulin molecule that is derived from any species (e.g., human, rodent, mouse, rat, camelid). The antigen-binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an anti-TROP2/EGFR antibody or antigen binding fragment thereof can comprise e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen-binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv in an anti-TROP2/EGFR antibody has two heavy chain variable domains, and two light chain variable domains. In some embodiments, the anti-TROP2/EGFR scFv has two antigen binding regions (Antigen binding regions: A and B), and the two antigen binding regions can bind to the respective target antigens with different affinities.

In some embodiments, the anti-TROP2/EGFR antibodies or antigen-binding fragments thereof can comprises one, two, or three heavy chain variable region CDRs selected from FIGS. 4-5. In some embodiments, the anti-TROP2/EGFR antibodies or antigen-binding fragments thereof can comprises one, two, or three light chain variable region CDRs selected from FIG. 6.

In some embodiments, the anti-TROP2/EGFR antibodies described herein can be conjugated to a therapeutic agent. The anti-TROP2/EGFR antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicine, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF. In some embodiments, the therapeutic agent is conjugated via a linker, e.g., a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

In some embodiments, the anti-TROP2/EGFR antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-TROP2/EGFR antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-TROP2/EGFR antibody or an antigen-binding fragment in vitro (e.g., in tissue culture or when stored as a pharmaceutical composition) or in vivo (e.g., in a human).

The anti-TROP2/EGFR antibodies or antigen-binding fragments thereof can also have various forms. Many different formats of bispecific antibodies or antigen-binding fragments thereof are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the anti-TROP2/EGFR antibody is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the anti-TROP2/EGFR antibody is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a 1 mmTAC, an IgG-IgG conjugate, a Cov-X-Body, or a scFv1-PEG-scFv2.

In some embodiments, the anti-TROP2/EGFR antibody can be a TrioMab. In a TrioMab, the two heavy chains are from different species, wherein different sequences restrict the heavy-light chain pairing.

In some embodiments, the anti-TROP2/EGFR antibody has two different heavy chains and one common light chain. Heterodimerization of heavy chains can be based on the knobs-into-holes or some other heavy chain pairing technique.

In some embodiments, CrossMAb technique can be used produce bispecific anti-TROP2/EGFR antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multi-specific antibodies.” MAbs. Vol. 8. No. 6. Taylor & Francis, 2016, which is incorporated by reference in its entirety. In some embodiments, the CH1 in the heavy chain and the CL domain in the light chain are swapped.

The anti-TROP2/EGFR antibody can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the anti-TROP2/EGFR antibody can be have a IgG-IgG format. Two intact IgG antibodies are conjugated by chemically linking the C-terminals of the heavy chains.

The anti-TROP2/EGFR antibody can also have a Fab-scFv-Fc format. In Fab-scFv-Fc format, a light chain, heavy chain and a third chain containing the Fc region and the scFv are assembled. It can ensure efficient manufacturing and purification.

In some embodiments, the anti-TROP2/EGFR antibody can be a TF. Three Fab fragments are linked by disulfide bridges. Two fragments target the tumor associated antigen (TAA) and one fragment targets a hapten. The TF format does not have an Fc region.

ADAPTIR has two scFvs bound to each side of an Fc region. It abandons the intact IgG as a basis for its construct, but conserves the Fc region to extend the half-life and facilitate purification.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The scFv-scFv-toxin includes toxin and two scFv with a stabilizing linker. It can be used for specific delivery of payload.

In some embodiments, the anti-TROP2/EGFR antibody is a bispecific antibody. In some embodiments, the bispecific antibody in present disclosure is designed to be 1+1 (monovalent for each target) and has an IgG1 subtype structure. This can reduce the avidity to cells with low expression levels of EGFR and TROP2, and increase the avidity to cells that co-express EGFR and TROP2, to achieve enhanced targeting function.

In some embodiments, the anti-TROP2/EGFR antibodies or antigen-binding fragments thereof have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 26, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 27 and 28.

In some embodiments, the anti-TROP2/EGFR antibodies include KIH mutations. In some embodiments, the anti-TROP2/EGFR antibody includes a first antigen-binding domain that specifically binds to EGFR, and a second antigen-binding domain that specifically binds to TROP2. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that including one or more hole mutations (a hole heavy chain), and the second antigen-binding domain includes a heavy chain including one or more knob mutations (a knob heavy chain). In some embodiments, the anti-TROP2/EGFR antibody includes a knob heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 27. In some embodiments, the anti-TROP2/EGFR antibody includes a hole heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 28.

### Antibody and ADC Characteristics

The anti-TROP2/EGFR antibodies can include an anti-EGFR antigen-binding domain and any anti-TROP2 antigen-binding domain as described herein.

The disclosure provides anti-TROP2/EGFR antibodies and antigen-binding fragments thereof that can specifically bind to EGFR. These anti-TROP2/EGFR antibodies can be agonists or antagonists. The anti-TROP2/EGFR antibodies, or antigen-binding fragments thereof described herein can bind to EGFR, and block the binding between EGFR and EGF, and/or the binding between EGFR and TGFα. By blocking the binding between EGFR and EGF, and/or the binding between EGFR and TGFα, the anti-TROP2/EGFR antibodies can inhibit the EGFR-associated signaling pathway and thus treating cancer (e.g., NSCLC). In some embodiments, the anti-TROP2/EGFR antibodies, or antigen-binding fragments thereof can initiate CMC or ADCC.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the anti-TROP2/EGFR antibodies or antigen-binding fragments thereof can bind to EGFR (e.g., human EGFR, monkey EGFR, mouse EGFR, and/or chimeric EGFR) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

In some embodiments, the anti-TROP2/EGFR antibodies or antigen-binding fragments thereof can bind to EGFR (e.g., human EGFR, monkey EGFR, mouse EGFR, and/or chimeric EGFR) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

The anti-TROP2/EGFR antibodies or antigen-binding fragments thereof can also include an antigen-binding domain that can specifically bind to TROP2. The anti-TROP2/EGFR antibodies or antigen-binding fragments thereof described herein can block the binding between TROP2 and its ligands (e.g., claudin-1, claudin-7, cyclin D1, and IGF-1). In some embodiments, by binding to TROP2, the anti-TROP2/EGFR antibody can also inhibit TROP2-associated signaling pathways, thereby inhibiting cell proliferation, differentiation, and/or metastasis. Thus, in some embodiments, the anti-TROP2/EGFR antibodies as described herein are TROP2 agonist. In some embodiments, the anti-TROP2/EGFR antibodies are TROP2 antagonist.

In some implementations, the anti-TROP2/EGFR antibodies or antigen-binding fragments thereof can bind to TROP2 (e.g., human TROP2, monkey TROP2, mouse TROP2, and/or chimeric TROP2) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

Because the anti-TROP2/EGFR antibody (e.g., bispecific antibody) binds to both TROP2 and EGFR, for cells that express both TROP2 and EGFR, the antibody has a higher binding affinity to these cells. Avidity can be used to measure the binding affinity of an antibody to these cells. Avidity is the accumulated strength of multiple affinities of individual non-covalent binding interactions.

In some embodiments, the anti-TROP2/EGFR antibody or ADC described herein can bind to cells expressing TROP2 and/or EGFR (e.g., A431 cells or human lung cancer HCC827 cells) with a EC50 value that is less than 3 nM, less than 2.5 nM, less than 2 nM, less than 1.9 nM, less than 1.8 nM, less than 1.7 nM, less than 1.6 nM, or less than 1.5 nM.

Thermal stabilities can also be determined. The anti-TROP2/EGFR antibodies or antigen-binding fragments thereof as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicate the denaturation of the Fc domains (Tm D1) and Fab domains (Tm D2), respectively. When there are two peaks, Tm usually refers to Tm D2. Thus, in some embodiments, the anti-TROP2/EGFR antibodies or antigen-binding fragments thereof as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the anti-TROP2/EGFR antibodies or antigen-binding fragments thereof as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the anti-TROP2/EGFR antibodies or antigen-binding fragments thereof can bind to human EGFR or monkey EGFR. In some embodiments, the anti-TROP2/EGFR antibodies or antigen-binding fragments thereof cannot bind to human EGFR or monkey EGFR. In some embodiments, the anti-TROP2/EGFR antibodies or antigen-binding fragments thereof can bind to human TROP2 or monkey TROP2. In some embodiments, the anti-TROP2/EGFR antibodies or antigen-binding fragments thereof cannot bind to human TROP2 or monkey TROP2.

In some embodiments, the anti-TROP2/EGFR antibody, antigen-binding fragment, or ADC has a purity that is greater than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC. In some embodiments, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC.

In some embodiments, the anti-TROP2/EGFR antibody, antigen-binding fragment, or ADC has a tumor growth inhibition rate or percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the anti-TROP2/EGFR antibody, antigen-binding fragment, or ADC has a tumor growth inhibition percentage that is less than 60%, 70%, 80%, 90%, 100%, 110%, 120%, 130%, 140%, or 150%. The TGI (%) can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 days after the treatment starts. As used herein, the tumor growth inhibition rate or percentage (TGI %) is calculated using the following formula:

TGI (%)=[1−(Ti−T0)/(Vi−V0)]×100%

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the anti-TROP2/EGFR antibody, antigen-binding fragment, or ADC has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the anti-TROP2/EGFR antibody, antigen-binding fragment, or ADC does not have a functional Fc region. For example, the anti-TROP2/EGFR antibodies or antigen-binding fragments thereof are Fab, Fab′, F(ab′)₂, and Fv fragments. In some embodiments, the anti-TROP2/EGFR antibodies or antigen-binding fragments thereof as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the main peak of HPLC-SEC accounts for at least 70%, 75%, 80%, 85%, 90%, 95%, 96%, 97%, 98%, 99%, 99.5%, 100% of the protein complex described herein after purification by protein A-based affinity chromatography and/or size-exclusive chromatography.

In some embodiments, the anti-TROP2/EGFR ADC described herein has an IC50 for in vitro killing of cancer cells (e.g., human epidermoid carcinoma cell line A431, human breast cancer cell line MCF-7, human lung cancer cell line NCI-H226 or NCI-H520) of less than 5 μg/mL, less than 4.5 μg/mL, less than 4 μg/mL, less than 3.5 μg/mL, less than 3 μg/mL, less than 2.5 μg/mL, less than 2 μg/mL, less than 1.5 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, less than 0.1 μg/mL, less than 0.05 μg/mL, less than 0.025 μg/mL, less than 0.0125 μg/mL, less than 0.005 μg/mL, or less than 0.0025 μg/mL. In some embodiments, the anti-TROP2/EGFR ADC described herein has an IC50 for in vitro killing of cancer cells (e.g., HCC827 cells, NCI-H292 cells, A431 cells, or Panc 02.03 cells) of less than 15 μg/mL, less than 10 μg/mL, less than 5 μg/mL, less than 1 μg/mL, less than 0.9 μg/mL, less than 0.8 μg/mL, less than 0.7 μg/mL, less than 0.6 μg/mL, or less than 0.5 μg/mL.

In some embodiments, the anti-TROP2/EGFR antibody or ADC described herein has a higher endocytosis rate than the corresponding monoclonal antibodies and/or control bispecific antibodies described herein. In some embodiments, the anti-TROP2/EGFR antibody or ADC described herein has a higher endocytosis rate than Cetuximab analog. In some embodiments, the anti-TROP2/EGFR antibody or ADC described herein has a higher endocytosis rate than DS-1062 analog and/or Sacituzumab analog. In some embodiments, the bispecific anti-TROP2/EGFR antibody or ADC described herein has a higher endocytosis rate than Amivantamab analog. In some embodiments, the anti-TROP2/EGFR ADC described herein has a higher endocytosis rate than an isotype control ADC (e.g., ISO-CPT2).

In some embodiments, the anti-TROP2/EGFR ADC described herein has a half-life of at least 3 days, at least 4 days, at least 5 days, at least 6 days, at least 7 days, at least 8 days, at least 9 days, at least 10 days, at least 11 days, at least 12 days, or at least 13 days, when administer at 1-20 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg) and detected 15 minutes, 2 hours, 6 hours, 1 day, 3 days, 5 days, 7 days, 10 days, 14 days or 21 days after administration. In some embodiments, the anti-TROP2/EGFR ADC described herein has a half-life that is at least 60%, at least 70%, at least 80%, at least 90%, at least 100%, at least 110%, at least 120%, at least 130%, or at least 140% as compared to an isotype control ADC (e.g., ISO-ADC). In some embodiments, the anti-TROP2/EGFR ADC described herein has a clearance rate of less than 25 mL/day/kg, 24 mL/day/kg, 23 mL/day/kg, 22 mL/day/kg, 21 mL/day/kg, 20 mL/day/kg, 19 mL/day/kg, 18 mL/day/kg, 17 mL/day/kg, 16 mL/day/kg, 15 mL/day/kg, 14 mL/day/kg, 13 mL/day/kg, 12 mL/day/kg, 11 mL/day/kg, or 10 mL/day/kg. In some embodiments, the PK profile of the anti-TROP2/EGFR ADC described herein is determined based on either the serum concentration of the administered anti-TROP2/EGFR ADC, or the total antibody derived from the anti-TROP2/EGFR ADC.

In some embodiments, the ratio of free therapeutic agent (e.g., MMAE or CPTx) derived from the administered anti-TROP2/EGFR ADC described herein is less than 10%, less than 5%, less than 4%, less than 3%, less than 2%, or less than 1%, after the anti-TROP2/EGFR ADC is added to plasma (e.g., human, monkey, or rat plasma) for at least 1 day, 2 days, 6 days, 8 days, 11 days, 14 days, 18 days, or 21 days. In some embodiments, the terminal concentration of the ADC is about 10-500 μg/mL (e.g., 100 μg/mL).

### Antibody Drug Conjugates (ADC)

The anti-TROP2/EGFR antibodies or antigen-binding fragments thereof described herein can be conjugated to a therapeutic agent (a drug). The therapeutic agent can be covalently or non-covalently bind to the anti-TROP2/EGFR antibody. In some embodiments, the anti-TROP2/EGFR antibody is an anti-TROP2/EGFR bispecific antibody. In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicine, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

Definitions of specific functional groups and chemical terms are described in more detail below. For purpose of this invention, the chemical elements are identified in accordance with the Periodic Table of the Elements, CAS version, Handbook of Chemistry and Physics, 75^{th} Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

All ranges cited herein are inclusive, unless expressly stated to the contrary. When a range of values is listed, it is intended to encompass each value and sub-range within the range. For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The compounds or any formula depicting and describing the compounds of the present disclosure may have one or more chiral (asymmetric) centers. The present invention encompasses all stereoisomeric forms of the compounds or any formula depicting and describing the compounds of the present invention. Centers of asymmetry that are present in the compounds or any formula depicting and describing the compounds of the present invention can all independently of one another have (R) or (S) configuration. When bonds to a chiral carbon are depicted as straight lines in the structural formulas, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the formula or by the name.

The disclosure includes all possible enantiomers and diastereomers and mixtures of two or more stereoisomers, for example mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism the disclosure includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds or any formula depicting and describing the compounds of the present invention. Examples of isotopes suitable for inclusion in the compounds of the present invention include, but not limited to, isotopes of hydrogen, such as ²H (i.e., D) and ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopic variants of the compounds or any formula depicting and describing the compounds of the present disclosure, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (²H, or D), can afford certain therapeutic advantages, for example, resulting from greater metabolic stability, increased in vivo half-life, or reduced dosage requirements and, hence, may be utilized in some particular circumstances. Isotopic variants of compounds or any formula depicting and describing the compounds of the present disclosure can generally be prepared by techniques known to those skilled in the art or by processes analogous to those described in the accompanying examples and synthesis using an appropriate isotopically-labeled reagent in place of the non-labeled reagent previously employed.

The compounds as provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present disclosure contain one or more acidic or basic groups, the disclosure also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present disclosure include but are not limited to copper (I), copper (II), iron (II), iron (III), manganese (II), and zinc salts. Compounds of the present disclosure which contain one or more basic groups, e.g., groups which can be protonated, can be present in salt form, and can be used according to the disclosure in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the disclosure may moreover be an integral or non-integral multiple of one.

Compounds of the present disclosure which contain basic nitrogen-containing groups can be quaternized using agents such as C₁₋₄alkyl halides, for example, methyl, ethyl, isopropyl, and tert-butyl chloride, bromide, and iodide; diC₁₋₄alkyl sulfates, for example, dimethyl, diethyl, and diamyl sulfate; C₁₀₋₁₈alkyl halides, for example, decyl, dodecyl, lauryl, myristyl, and stearyl chloride, bromide, and iodide; and arylC₁₋₄alkyl halides, for example, benzyl chloride and phenethyl bromide.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound or any formula depicting and describing the compounds of the present disclosure and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present disclosure may include those wherein the solvent of crystallization may be isotopically substituted, e.g., D₂O, d₆-acetone, d₆-DMSO.

### Linker (Linking Agent Compound)

In some embodiments, the therapeutic agent is conjugated via a linker (or a linking agent compound). As used herein, the term “linker” or “linking agent compound” refers to a compound that can connect a ligand (e.g., the antibodies or the antigen-binding fragments thereof described herein) and a therapeutic agent (e.g., any of the therapeutic agents described herein) together to form a ligand-drug conjugate by reacting with a group of the ligand compound and the therapeutic agent compound respectively by, for example, a coupling reaction.

In some embodiments, the linker described herein is a compound having the following formula:

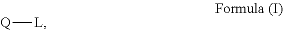

or a pharmaceutically acceptable salt, solvate, stereoisomer, or isotopic variant thereof, wherein Q denotes to a junction moiety capable of being coupled to a ligand via a bond selected from the group consisting of carbonyl, thioether, amide, disulfide and hydrazone bond; L denotes to a linker moiety capable of connecting Q to a therapeutic agent.

In some embodiments, the junction moiety (Q in Formula (I)) has the following structure:

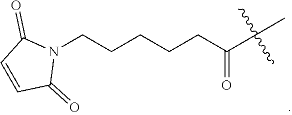 [SMILES: *C(=O)CCCCCN1C(=O)C=CC1=O]

In some embodiments, the linker moiety (L in Formula (I)) has the following formula:

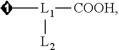

where L₁ is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O) (CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and
[symbol] denotes to the N-terminal side of the polypeptide residue covalently attached to the junction moiety Q.

In some embodiments, the polypeptide residue L₁ is ^{NH}-Glu-Val-Ala-^{COOH}. In some embodiments, the hydrophilic group L₂ has the following structure:

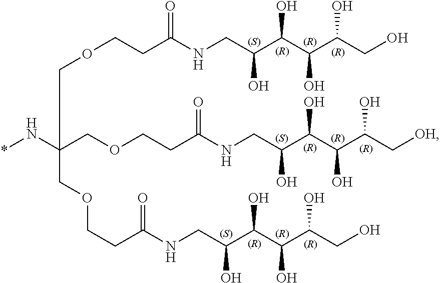

wherein “*” denotes the site covalently attached to polypeptide residue L₁, e.g., side chain of the Glu residue in ^{NH}-Glu-Val-Ala-^{COOH}.

In some embodiments, the linker described herein is a compound having the following structure:

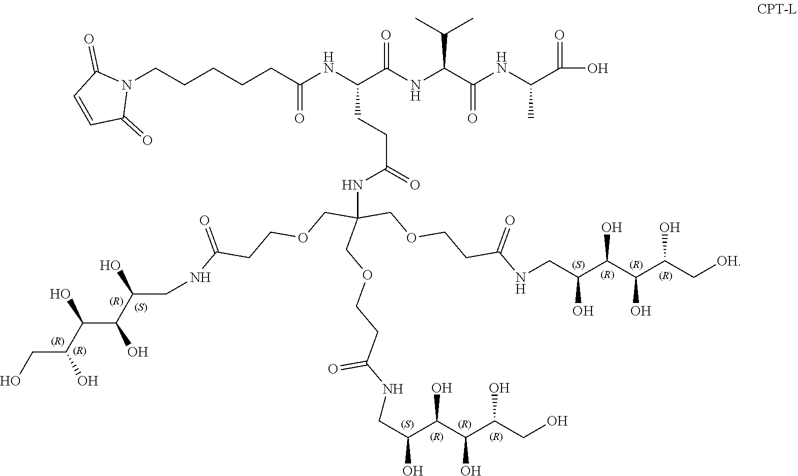

CPT-L is an exemplary linker for coupling therapeutic agent CPT-1/2/3/4 as described in the present disclosure.

In some embodiments, the linker is a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

### Therapeutic Agent

In some embodiments, the therapeutic agent that is conjugated to the antibodies or the antigen-binding fragments thereof described herein is discussed as follows.

In some embodiments, the therapeutic agent described herein is a cytotoxic agent. In some embodiments, the cytotoxic agent is a camptothecin compound, an analogue or a derivative thereof. In some preferred embodiments, the camptothecin compound is a compound having the following structure:

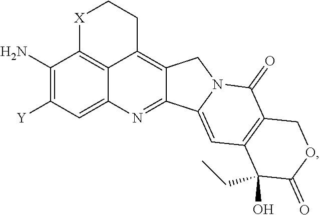

wherein X is selected from the group consisting of —CH2-, O and S; Y is selected from the group consisting of H, D, and F.

In some embodiments, the therapeutic agent is (S)-4-amino-9-ethyl-9-hydroxy-1,9,12,15-tetrahydro-13H-pyrano[3′,4′:6,7]indolizino[1,2-b]thiopyrano[4,3,2-de]quinoline-10,13(2H)-dione) (CPT-1). The structure of CPT-1 is shown below:

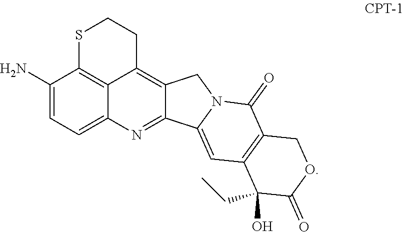 [SMILES: CC[C@@]1(O)C(=O)OCc2c1cc1n(c2=O)Cc2c-1nc1ccc(N)c3c1c2CCS3]

In some embodiments, the therapeutic agent is (S)-4-amino-9-ethyl-9-hydroxy-1,9,12,15-tetrahydro-13H-pyrano[4,3,2-de]pyrano[3′,4′:6,7]indolizino[1,2-b]quinoline-10,13(2H)-dione (CPT-2). The structure of CPT-2 is shown below:

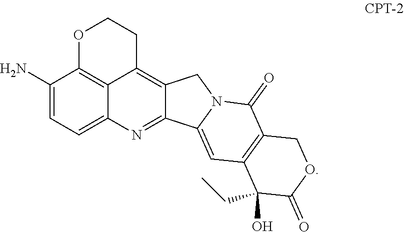 [SMILES: CC[C@@]1(O)C(=O)OCc2c1cc1n(c2=O)Cc2c-1nc1ccc(N)c3c1c2CCO3]

In some embodiments, the therapeutic agent is CPT3. The structure of CPT-3 is shown below:

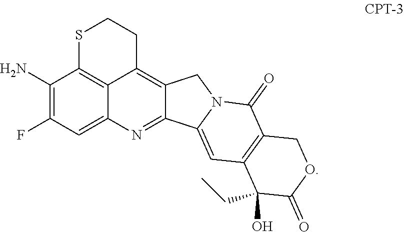 [SMILES: CC[C@@]1(O)C(=O)OCc2c1cc1n(c2=O)Cc2c-1nc1cc(F)c(N)c3c1c2CCS3]

In some embodiments, the therapeutic agent is (S)-4-amino-9-ethyl-5-fluoro-9-hydroxy-1,9,12,15-tetrahydro-13H-pyrano[4,3,2-de]pyrano[3′,4′:6,7]indolizino[1,2-b]quinoline-10,13(2H)-dione (CPT-4). The structure of CPT-4 is shown below:

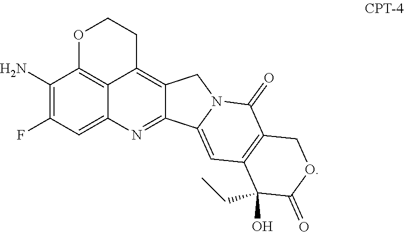 [SMILES: CC[C@@]1(O)C(=O)OCc2c1cc1n(c2=O)Cc2c-1nc1cc(F)c(N)c3c1c2CCO3]

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

### Linker-Therapeutic Agent Compound

In some embodiments, a linker (e.g., any of the linkers described herein) and a therapeutic agent (e.g., any of the therapeutic agents described herein) can be linked to form a “linker-therapeutic agent” compound. Several specific examples of methods of preparing “linker-therapeutic agent” compounds are known in the art and are described in PCT/CN2023/093976, PCT/CN2023/117368, PCT/CN2023/120684, and PCT/CN2024/092145; which are incorporated herein by reference in the entirety.

In some embodiments, the linker-therapeutic agent compound has the following structure:

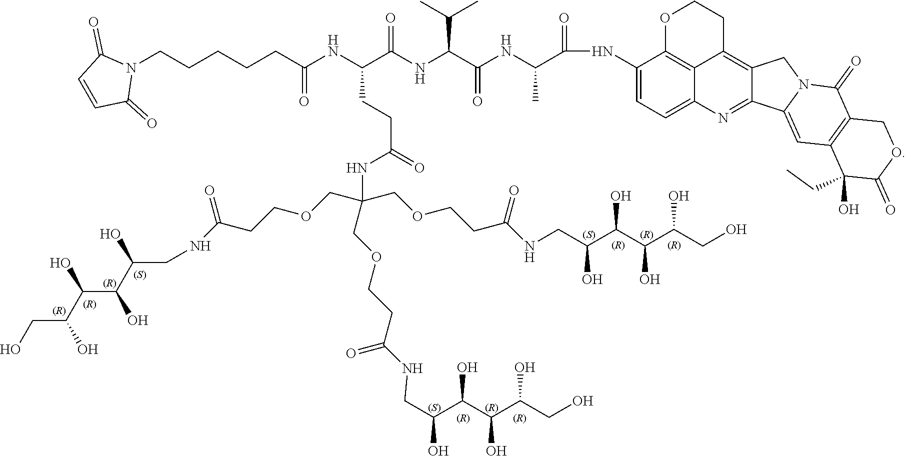

In some embodiments, the linker-therapeutic agent compound has the following structure:

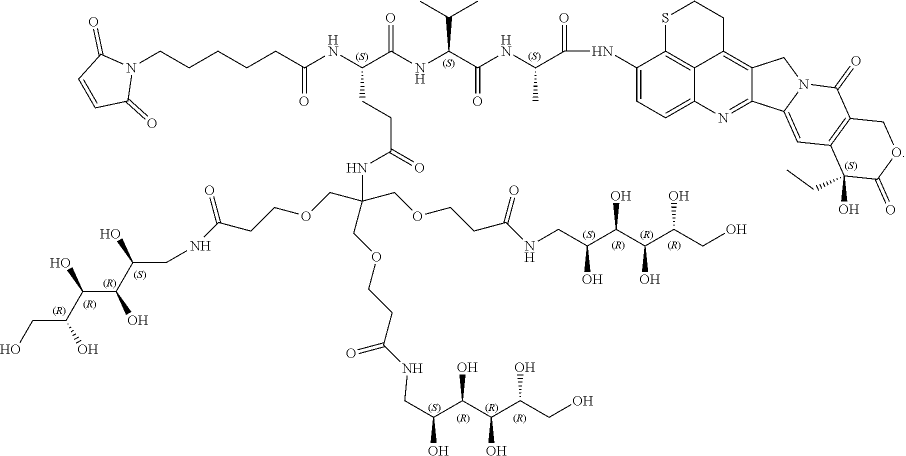

In some embodiments, an antibody (“Ab”), e.g., any of the antibodies or the antigen-binding fragments thereof described herein, can be linked to a linker-therapeutic agent compound (e.g., any of the linker-therapeutic agent compounds described herein) to generate an antibody-drug conjugate. In some embodiments, the antibody-drug conjugate has the following structure:

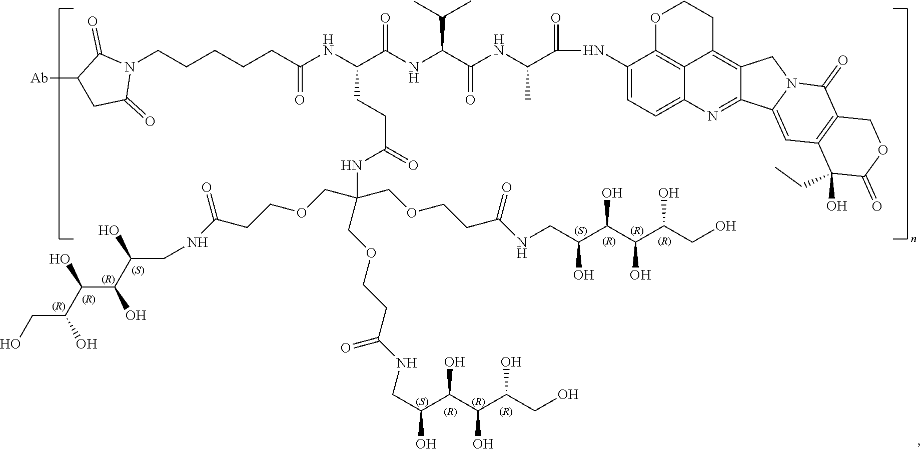

wherein n=1-8. In some embodiments, n=1-8. In some embodiments, n is about 1, about 2, about 3, about 4, about 5, about 6, about 7, about 8. In some embodiments, n is about 1-8, 1-7, 1-6, 1-5, 1-4, 1-3, 1-2, 2-8, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8. In some embodiments, n is an integral or non-integral multiple of one.

In some embodiments, the anti-TROP2/EGFR antibody is coupled to the drug via a cleavable linker e.g. a SPBD linker or a maleimidocaproyl-valine-citrulline-p-aminobenzyloxycarbonyl (VC) linker. In some embodiments, the anti-TROP2/EGFR antibody is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the anti-TROP2/EGFR antibody, any structural constraints of the drug and the hydrophobicity of the drug (see, for example, review in Nolting, Chapter 5, Antibody-Drug Conjugates: Methods in Molecular Biology, 2013, Ducry (Ed.), Springer). A number of specific linker-toxin combinations have been described and may be used with the anti-TROP2/EGFR antibodies or antigen-binding fragments thereof described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the anti-TROP2/EGFR antibodies or antigen-binding fragments thereof through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The anti-TROP2/EGFR ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the anti-TROP2/EGFR ADCs described here. Various prepared linkers, linker components and toxins are commercially available or may be prepared using standard synthetic organic chemistry techniques. These methods are described e.g., in March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected anti-TROP2/EGFR antibody or antigen-binding fragment are also available commercially, for example, linker-toxins comprising DM1, DM4, MMAE, MMAF or Duocarmycin SA are available from Creative BioLabs (Shirley, N.Y.).

Several specific examples of methods of preparing anti-TROP2/EGFR ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an anti-TROP2/EGFR antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the DAR for an anti-TROP2/EGFR ADC composition is about or at least 1, 2, 3, 4, 5, 6, 7, or 8. In some embodiments, the average DAR in the anti-TROP2/EGFR ADC composition is about 1˜about 2, about 2˜about 3, about 3˜about 4, about 3˜about 5, about 4˜about 5, about 5˜about 6, about 6˜about 7, or about 7˜about 8.

In some embodiments, anti-TROP2/EGFR antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the anti-TROP2/EGFR antibody can be further engineered to replace the Asparagine at position 297 with Alanine (N297A).

In some embodiments, to facilitate production efficiency by avoiding Fab-arm exchange, the Fc region of the anti-TROP2/EGFR antibodies or antigen-binding fragments thereof was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific anti-TROP2/EGFR antibody. Bispecific anti-TROP2/EGFR antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knobs-into-holes (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of anti-TROP2/EGFR antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an anti-TROP2/EGFR antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., an anti-TROP2/EGFR antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to EGFR. In some embodiments, a pair of vectors is provided, wherein each vector comprises one of the nucleic acids as described herein, wherein together the pair of vectors encodes the VL region and the VH region that together bind to EGFR.

In some embodiments, the vector includes two of the nucleic acids as described herein, wherein the vector encodes the VL region and the VH region that together bind to TROP2. In some embodiments, a pair of vectors is provided, wherein each vector comprises one of the nucleic acids as described herein, wherein together the pair of vectors encodes the VL region and the VH region that together bind to TROP2.

### Methods of Treatment

The methods described herein include methods for the treatment of disorders associated with cancer. Generally, the methods include administering a therapeutically effective amount of anti-TROP2/EGFR antibodies or anti-TROP2/EGFR antibody-drug conjugates as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of anti-TROP2/EGFR antibodies or an anti-TROP2/EGFR antibody drug conjugates disclosed herein to a subject in need thereof, e.g., a subject having, or identified or diagnosed as having, a cancer, e.g., solid tumor, lung cancer (e.g., non-small cell lung cancer, lung adenocarcinoma, or lung carcinoma), gastric cancer (e.g., gastric carcinoma), skin cancer (e.g., skin carcinoma), colorectal cancer, breast cancer, head and neck cancer, ovarian cancer, prostate cancer, thyroid cancer, pancreatic cancer, CNS cancer, liver cancer, nasopharynx cancer, brain cancer, colon cancer, bladder cancer, oral squamous cell carcinoma, cervical cancer, or oesophageal cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the anti-TROP2/EGFR antibody, anti-TROP2/EGFR antigen binding fragment, anti-TROP2/EGFR antibody-drug conjugates, anti-TROP2/EGFR antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an anti-TROP2/EGFR antibody, an anti-TROP2/EGFR antigen binding fragment, or an anti-TROP2/EGFR antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an anti-TROP2/EGFR antibody, anti-TROP2/EGFR antigen binding fragment, or anti-TROP2/EGFR antibody-drug conjugate may vary, depending on, inter alia, patient history as well as other factors such as the type (and/or dosage) of the agent used.

Effective amounts and schedules for administering the anti-TROP2/EGFR antibodies, anti-TROP2/EGFR antigen-binding fragments thereof, anti-TROP2/EGFR antibody-encoding polynucleotides, anti-TROP2/EGFR antibody-drug conjugates, and/or compositions disclosed herein may be determined empirically, and making such determinations is within the skill in the art. Those skilled in the art will understand that the dosage that must be administered will vary depending on, for example, the mammal that will receive the anti-TROP2/EGFR antibodies, anti-TROP2/EGFR antigen-binding fragments thereof, anti-TROP2/EGFR antibody-encoding polynucleotides, anti-TROP2/EGFR antibody-drug conjugates, and/or compositions disclosed herein, the route of administration, the particular type of the agent or compositions disclosed herein used and other drugs being administered to the mammal.

A typical daily dosage of an effective amount of an anti-TROP2/EGFR antibody or anti-TROP2/EGFR ADC is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one anti-TROP2/EGFR antibody, the anti-TROP2/EGFR antigen-binding fragment thereof, anti-TROP2/EGFR antibody-drug conjugates, or pharmaceutical composition (e.g., comprising any of the anti-TROP2/EGFR antibodies, anti-TROP2/EGFR antigen-binding antibody fragments, or anti-TROP2/EGFR ADC) and, optionally, at least one additional therapeutic agent can be administered to the subject (e.g., once a week, twice a week, three times a week, four times a week, once a day, twice a day, or three times a day).

In some embodiments, the one or more additional therapeutic agents can be administered to the subject prior to, or after administering the at least one anti-TROP2/EGFR antibody, anti-TROP2/EGFR antigen-binding antibody fragment, anti-TROP2/EGFR antibody-drug conjugate, or pharmaceutical composition (e.g., comprising any of the anti-TROP2/EGFR antibodies, anti-TROP2/EGFR antigen-binding antibody fragments, or anti-TROP2/EGFR ADC). In some embodiments, the one or more additional therapeutic agents and the at least one anti-TROP2/EGFR antibody, anti-TROP2/EGFR antigen-binding antibody fragment, or anti-TROP2/EGFR antibody-drug conjugate are administered to the subject such that there is an overlap in the bioactive period of the one or more additional therapeutic agents and the at least one anti-TROP2/EGFR antibody, anti-TROP2/EGFR antigen-binding fragment, or anti-TROP2/EGFR ADC in the subject.

In some embodiments, the subject can be administered the at least one anti-TROP2/EGFR antibody, anti-TROP2/EGFR antigen-binding antibody fragment, anti-TROP2/EGFR antibody-drug conjugate, or pharmaceutical composition (e.g., comprising any of the anti-TROP2/EGFR antibodies, anti-TROP2/EGFR antigen-binding antibody fragments, or anti-TROP2/EGFR ADC) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of anti-TROP2/EGFR antibodies or anti-TROP2/EGFR antigen-binding antibody fragments, anti-TROP2/EGFR antibody-drug conjugates (and/or one or more additional therapeutic agents) administered to the subject and can also adjust (e.g., increase or decrease) the dosage or frequency of administration of at least one anti-TROP2/EGFR antibody, anti-TROP2/EGFR antigen-binding antibody fragment, or anti-TROP2/EGFR ADC (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of TROP2, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomib, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, anti-PD-L2 antibody, an anti-LAG-3 antibody, an anti-TIGIT antibody, an anti-BTLA antibody, an anti-CTLA4 antibody, an anti-CD40 antibody, an anti-OX40 antibody, an anti-4-1BB antibody, an anti-TIM3 antibody, or an anti-GITR antibody.

### Pharmaceutical Compositions and Routes of Administration

Also provided herein are pharmaceutical compositions that contain at least one (e.g., one, two, three, or four) of the anti-TROP2/EGFR antibodies (e.g., bispecific antibodies), anti-TROP2/EGFR antigen-binding fragments, or anti-TROP2/EGFR antibody-drug conjugates described herein. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the anti-TROP2/EGFR antibody, anti-TROP2/EGFR antigen-binding fragment thereof, or the anti-TROP2/EGFR ADC can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the anti-TROP2/EGFR antibodies, anti-TROP2/EGFR antigen-binding fragments, anti-TROP2/EGFR antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the anti-TROP2/EGFR antibodies, an anti-TROP2/EGFR antigen-binding fragment thereof, or an anti-TROP2/EGFR ADC will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the anti-TROP2/EGFR antibodies, the anti-TROP2/EGFR antigen-binding fragment thereof, or the anti-TROP2/EGFR ADC described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the anti-TROP2/EGFR antibodies, the anti-TROP2/EGFR antigen-binding fragments thereof, or the anti-TROP2/EGFR ADCs described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). In some embodiments, the dose level is between 5-30 mg/kg, 5-25 mg/kg, 5-20 mg/kg, 5-15 mg/kg, 5-10 mg/kg, 10-30 mg/kg, 10-25 mg/kg, 10-20 mg/kg, 10-15 mg/kg, 15-30 mg/kg, 15-25 mg/kg, 15-20 mg/kg, 20-30 mg/kg, 20-25 mg/kg, or 25-30 mg/kg. In some embodiments, the dose level is about 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, or 30 mg/kg. In some embodiments, the dose levels described herein do not induce severe toxic effects to the subject. While these doses cover a broad range, one of ordinary skill in the art will understand that therapeutic agents vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the therapeutic agent in vivo.

The pharmaceutical compositions can be included in a container, pack, or dispenser together with instructions for administration. The disclosure also provides methods of manufacturing the anti-TROP2/EGFR antibodies, the anti-TROP2/EGFR antigen-binding fragment thereof, or the anti-TROP2/EGFR ADC for various uses as described herein.

### EXAMPLES

The invention is further described in the following examples, which do not limit the scope of the invention described in the claims.

### Example 1. Preparation and Analysis of Anti-TROP2/EGFR Bispecific Antibodies

Provided herein are bispecific antigen-binding molecules targeting TROP2 and EGFR. These antigen-binding molecules are referred to as anti-TROP2/EGFR bispecific antibodies below.

### Preparation of Anti-TROP2/EGFR Bispecific Antibodies

Anti-TROP2/EGFR bispecific antibodies can have anti-TROP2 antigen binding domains (T-6F7, VH: SEQ ID NO: 25, VL: SEQ ID NO: 22) and anti-EGFR antigen binding domains (E-1G11, VH: SEQ ID NO: 23, VL: SEQ ID NO: 22; or E-6C4, VH: SEQ ID NO: 24, VL: SEQ ID NO: 22). These antigen binding domains can be paired to form bispecific antibodies. Vectors encoding the light chain and heavy chain of the anti-TROP2/EGFR antibodies were constructed. CHO-S cells were co-transduced with three vectors, including a first vector encoding the heavy chain of an anti-TROP2 binding arm, a second vector encoding the heavy chain of an anti-EGFR binding arm, and a third vector encoding the common light chain. After 14 days of culture, the cell supernatant was collected and purified by Protein A affinity chromatography.

Various methods can be used to reduce the chance of mispairing between the two heavy chains. For example, knobs-into-holes mutations were introduced in the Fc regions of the anti-TROP2 arm heavy chain and the anti-EGFR arm heavy chain. Exemplary bispecific antibodies obtained include T-6F7-E-1G11 and T-6F7-E-6C4. To verify the binding affinity of bispecific antibodies, anti-TROP2 or anti-EGFR control bispecific antibodies were also generated, in which one arm of the control bispecific antibody recognizes TROP2 or EGFR, and the other arm recognizes CD28. Similar methods were used to generate these control bispecific antibodies, e.g., obtaining VH sequences by immunizing RenLite™ mice. Exemplary control bispecific antibodies are named as T-6F7-CD28, CD28-T-6F7, CD28-E-1G11, CD28-E-6C4, E-1G11-CD28 and E-6C4-CD28.

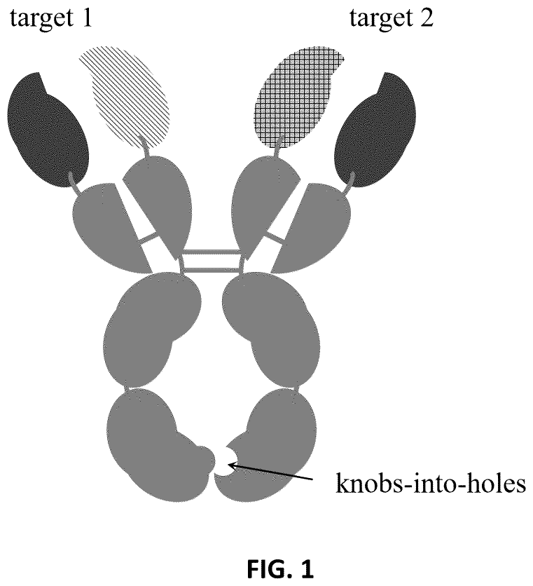

Knobs-into-holes mutations were introduced to all the bispecific antibodies. For example, in T-6F7-E-1G11, the heavy chain constant region of T-6F7 includes knob mutations, and the heavy chain constant region of E-1G11 includes hole mutations. In T-6F7-CD28, the heavy chain constant region of T-6F7 includes knob mutations, and the heavy chain constant region of CD28 includes hole mutations. An exemplary antibody structure is shown in FIG. 1, in which target 1 and target 2 can be TROP2 and EGFR, respectively; EGFR and TROP2, respectively; TROP2 and CD28, respectively; CD28 and TROP2, respectively; EGFR and CD28, or CD28 and EGFR, respectively.

The constant region can also include one or more mutations. For example, when SI mutations (EU numbering: S239D and I332E mutations) are introduced into the Fc region of T-6F7, the resulting antibody is named as T-6F7-SI.

The sequences of the light chain constant region, the heavy chain constant region with knob mutations, and the heavy chain constant region with hole mutations are shown in SEQ ID NO: 26, SEQ ID NO: 27 and SEQ ID NO: 28, respectively.

Internalization of Antibodies Targeting TROP2 and/or EGFR

Anti-TROP2 antibodies, anti-EGFR antibodies, anti-TROP2/EGFR bispecific antibodies, or anti-TROP2/CD28 bispecific antibodies together with the pHAb-Goat anti-human IgG secondary antibody were added to NCI-H292 cells (ATCC, Cat #: CRL-1848), respectively, and incubated for 1 hour. The cells were centrifuged and washed with FACS buffer. Mean fluorescence intensity (MFI) was measured using a flow cytometer. Endocytosis rates of antibodies were calculated. For isotype control (ISO), human IgG1 protein (hIgG1) was used. The results are shown in the following table.

TABLE 1

NCI-H292

| Antibodies | MFI | Positive Population |
|---|---|---|
| ISO | 6400 | 0.76% |
| Negative control | 5999 | 0.88% |
| Sacituzumab-SI analog | 86359 | 97.8% |
| Cetuximab analog | 17019 | 93.5% |
| T-6F7 | 35991 | 92.9% |
| E-1G11 | 13298 | 63.9% |
| E-6C4 | 12320 | 51.4% |
| T-6F7-CD28 | 6847 | 1.38% |
| E-6C4-CD28 | 9320 | 7.76% |
| E-1G11-CD28 | 9430 | 9.45% |
| T-6F7-E-6C4 | 41197 | 98.3% |
| T-6F7-E-1G11 | 27875 | 98.5% |

Cetuximab is an EGFR-targeting chimeric monoclonal IgG1 antibody originally developed by ImClone Systems and first launched in Switzerland in 2003 as Erbitux™ by Merck KGaA as a monotherapy and in combination with irinotecan for the treatment of irinotecan-refractory metastatic colorectal cancer. The heavy chain and light chain sequences of Cetuximab analog are shown in SEQ ID NO: 29 and SEQ ID NO: 30, respectively.

Sacituzumab-SI analog is a humanized anti-TROP2 monoclonal IgG1 antibody with the SI mutations in the constant region. The heavy chain and light chain sequences of Sacituzumab-SI analog are shown as SEQ ID NO: 31 and SEQ ID NO: 32, respectively.

The results showed that the bispecific antibodies T-6F7-E-6C4 and T-6F7-E-1G11 showed higher endocytosis rates than the corresponding monoclonal antibody T-6F7, E-6C4 or E-1G11. In addition, the control bispecific antibodies T-6F7-CD28, E-6C4-CD28 and E-1G11-CD28 showed lower endocytosis rates than the corresponding bispecific antibodies or monoclonal antibodies.

### Purity Analysis of Antibodies

Purified anti-TROP2/EGFR bispecific antibodies were analyzed by a non-reducing SDS-PAGE (sodium dodecyl sulphate-polyacrylamide gel electrophoresis) and SEC-HPLC (size exclusion chromatography-high performance liquid chromatography).

Non-reducing SDS-PAGE was performed using a 4-12% acrylamide gel. The protein samples were prepared as follows. First, 2.4 μL of the protein sample was mixed with 6 μL Tris-Glycine SDS Sample Buffer (2×) (Invitrogen; Cat #: LC2676) and 3.6 μL distilled water. The mixture was then boiled for 2 minutes and instantly centrifuged before loading. 4 μg of each sample was loaded to the gel.

In the SEC-HPLC method, the antibody samples were diluted to 1 mg/mL with PBS (pH 7.2-7.4, 0.01 M) and an Agilent 1290 chromatography system (connected with Xbridge™ Protein BEH SEC column (200 Å, Waters Corporation)) was used. The following parameters were used: mobile phase: 25 mmol/L phosphate buffer (PB)+300 mmol/L NaCl, pH 6.8; flow rate: 1.8 mL/min; column temperature: 25° C.; detection wavelength: 280 nm; injection volume: 10 mL; sample tray temperature: about 4° C.; and running time: 7 minutes. Results are summarized in the table below.

TABLE 2
Non-reducing SDS-PAGE and SEC-HPLC analysis results

| | Theoretical | Detected | | | Purified protein | SEC-HPLC |
|---|---|---|---|---|---|---|
| | molecular | molecular | | SDS-PAGE | expression level | Main peak |
| Sample | weight (KD) | weight (KD) | | result | (μg/mL) | (%) |
| T-6F7-SI | ~150 KD | ~180 | KD | Pass | 481.27 | 98.11% |
| E-6C4 | ~150 KD | ~180 | KD | Pass | 129.23 | 95.64% |
| E-1G11 | ~150 KD | ~180 | KD | Pass | 349.62 | 100% |
| CD28-T-6F7 | ~150 KD | ~180-245 | KD | Pass | 231.00 | 81.17% |
| CD28-E-6C4 | ~150 KD | ~180 | KD | Pass | 230.54 | 74.52% |
| E-1G11-CD28 | ~150 KD | ~180 | KD | Pass | 512.88 | 88.09% |
| T-6F7-E-6C4 | ~150 KD | ~180 | KD | Pass | 156.15 | 97.49% |
| T-6F7-E-1G11 | ~150 KD | ~180 | KD | Pass | 522.31 | 97.99% |

### Binding Affinities of Anti-TROP2/EGFR Bispecific Antibodies

The binding affinities of anti-TROP2/EGFR bispecific antibodies to human TROP2, human EGFR, monkey TROP2, and monkey EGFR were verified by surface plasmon resonance (SPR) using Biacore™ (Biacore, Inc., Piscataway N.J.) 8K biosensor equipped with pre-immobilized Protein A sensor chips.

Specifically, hTROP2-His (ACROBiosystems Inc., Cat #: TR2-H5223), hEGFR-His (ACROBiosystems Inc., Cat #: EGR-H5222), fasTROP2-His (ACROBiosystems Inc., Cat #: TR2-R52H3) and fasEGFR-His (ACROBiosystems Inc., Cat #: EGR-C52H1) were diluted to 200 nM with 1×HBS-EP+ buffer (pH 7.4). Purified antibodies were injected into the Biacore™ 8K biosensor at 10 mL/min for about 50 seconds to achieve a desired protein density (e.g., about 350 response units (RU)) and the diluted antigen protein at a concentration of 200 nM was then injected at 30 mL/min for 180 seconds. Dissociation was monitored for 400 seconds. The chip was regenerated after the last injection of each titration with a glycine solution (pH 1.5) at 30 mL/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 3
Affinity test results

| | Analyte 1 | kon | koff | KD | |
|---|---|---|---|---|---|
| | Solution | (1/Ms) | (1/s) | (M) | Results |
| T-6F7- | hTROP2-His | 1.08E+05 | 3.43E−04 | 3.18E−09 | positive |
| E-6C4 | fasTROP2-His | 1.12E+05 | 8.49E−04 | 7.62E−09 | positive |
| | hEGFR-His | 4.85E+05 | 4.13E−02 | 8.52E−08 | positive |
| | fasEGFR-His | 1.19E+05 | 6.95E−03 | 5.86E−08 | positive |
| T-6F7- | hTROP2-His | 1.09E+05 | 5.69E−04 | 5.24E−09 | positive |
| E-1G11 | fasTROP2-His | 9.49E+04 | 1.18E−03 | 1.24E−08 | positive |
| | hEGFR-His | 3.64E+04 | 1.50E−03 | 4.11E−08 | positive |
| | fasEGFR-His | 3.62E+04 | 1.42E−03 | 3.93E−08 | positive |

The results showed that the anti-TROP2/EGFR bispecific antibodies T-6F7-E-6C4 and T-6F7-E-1G11 had good binding affinity to human TROP2, monkey TROP2, human EGFR and monkey EGFR.

### Stability of Anti-TROP2/EGFR Bispecific Antibodies

Anti-TROP2/EGFR bispecific antibodies T-6F7-E-6C4 and T-6F7-E-1G11 were diluted to 5 mg/mL using a buffer (3 mg/mL histidine, 80 mg/mL sucrose, and 0.2 mg/mL Tween™ 80) at pH 6.0. The diluted antibodies were kept in sealed Eppendorf tubes at 4±3° C. (hereinafter referred to as 4° C.) for 7 days, or at 40±3° C. (hereinafter referred to as 40° C.) for 7 days, and their thermal stability was evaluated. Alternatively, the bispecific antibodies were also incubated at low pH conditions. Specifically, the antibodies were incubated in 1 mol/L acetic acid at pH 3.5 for 0 hour or 6 hours to determine its stability in acidic conditions.

After the above treatments, the following tests were performed: (1) observing the solution appearance and presence of visible non-soluble objects; (2) detecting the purity changes of antibodies by Size-Exclusion Ultra Performance Liquid Chromatography (SEC-UPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min)); (4) detecting the purity changes of antibodies by capillary electrophoresis-sodium dodecyl sulfate (CE-SDS) under non-reducing (CE-SDS(NR)) conditions (indicated as the percentage of the main peak area to the sum of all peak areas (Purity, %)); (5) detecting charge variants in the antibodies by the Capillary Isoelectric Focusing (Cief) method (indicated as the percentages of the main component, acidic component, and alkaline component).

In the SEC-UPLC experiments, the antibody samples were diluted to 1 mg/mL with purified water and an Agilent 1290 chromatography system (connected with Xbridge™ Protein BEH SEC column (200 Å, Waters Corporation)) was used. The following parameters were used: mobile phase: 100 mmol/L phosphate buffer (“PB”) (pH 7.4)+0.2 mol/L NaCl+10% acetonitrile; flow rate: 1.8 mL/min; column temperature: 25° C.; detection wavelength: 280 nm; injection volume: 10 mL; sample tray temperature: about 6° C.; and running time: 7 minutes.

In the HIC-HPLC experiments, an Agilent 1260 chromatography system (connected with ProPac™ HIC-10 column (4.6×250 mm, Thermo Scientific)) was used, and samples were diluted using mobile phase A to 0.5 mg/mL. The following parameters were used: mobile phase A: 1.0 M PB, 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 mL; sample tray temperature: about 6° C.; and running time: 45 minutes.

In the Cief experiments, a Maurice Cief Method Development Kit (Protein Simple, Cat #: PS-MDK01-C) was used for sample preparation. Specifically, 40 μg protein sample was mixed with the following reagents in the kit: 1 mL Maurice Cief Pi Marker-4.05, 1 mL Maurice Cief Pi Marker-9.99, 35 mL 1% Methyl Cellulose Solution, 2 mL Maurice Cief 500 mM Arginine, 4 mL Ampholytes (Pharmalyte pH ranges 3-10), and water (added to make a final volume of 100 mL). On the Maurice analyzer (Protein Simple, Santa Clara, CA), Maurice Cief Cartridges (PS-MC02-C) were used to generate imaging capillary isoelectric focusing spectra. The sample was focused for a total of 10 minutes. The analysis software installed on the instrument was used to integrate the absorbance of the 280 nm-focused protein.

In the CE-SDS(NR) experiments, Maurice (Protein simple, Maurice™) and Maurice CE-SDS Size Application Kit (Protein simple, Cat #: PS-MAK02-S) were used. 54 mL Sample Buffer, 6 mL antibody sample, 2.4 mL 25× internal standard, 3 mL 250 nM Iodoacetamide (SIGMA, Cat #: 16125) were add to a microcentrifuge tube, followed by centrifugation at 3000 rpm for 1 minute and heating in a 70° C. water bath for 10 minutes. The samples were then cooled to room temperature followed by centrifugation at 10000 rpm for 3 minutes. Supernatant sample preparations were then transferred to a 96-well plate and tested in Maurice. The following parameters were used: injection voltage: 4.6 kV; injection time: 20 seconds; separation voltage: 5.75 kV; and separation time: 40 minutes.

Detailed results of anti-TROP2/EGFR bispecific antibodies are shown in the table below. The results showed that T-6F7-E-6C4 and T-6F7-E-1G11 had better stability as well as physical and chemical properties than other tested antibodies.

TABLE 4

| | | | | | | | | | Cief | | |
|---|---|---|---|---|---|---|---|---|---|---|---|
| | | | | CE-SDS (NR) | | | | | Acidic | Main | alkaline |
| | | SEC | HIC | | HL | HH | HHL | | peak | peak | peak |
| Antibody | Treatment | Purity(%) | (min) | Purity (%) | (%) | (%) | (%) | U | (%) | (%) | (%) |
| T-6F7-E-6C4 | 0 d | 95.25 | 12.65 | 94.3 | — | — | 1 | 4.7 | 25.5 | 63.8 | 10 |
| | 4° C. 7 d | 97.76 | 12.63 | 94.0 | — | — | 1.1 | 4.8 | 25.5 | 65.4 | 9.1 |
| | 40° C. 7 d | 97.63 | 12.66 | 96.7 | — | — | 1.4 | 1.9 | 32.0 | 58.8 | 9.0 |
| | Ph 3.5 0 h | 100 | 12.66 | 94.9 | — | — | 5.1 | — | 22.8 | 69.0 | 3.1 |
| | Ph 3.5 6 h | 100 | 12.73 | 100 | — | — | — | — | 40.3 | 51.8 | 6.5 |
| T-6F7-E-1G11 | 0 d | 97.63 | 11.10 | 92.2 | 2.0 | 0.7 | 0.9 | 4.3 | 26 | 69.4 | 4.6 |
| | 4° C. 7 d | 97.45 | 11.07 | 92.4 | 1.8 | 0.7 | 0.9 | 4.2 | 24.4 | 70.9 | 4.7 |
| | 40° C. 7 d | 97.36 | 11.04 | 91.4 | 1.8 | 0.6 | 1.2 | 5.0 | 28.1 | 65.5 | 5.7 |
| | Ph 3.5 0 h | 100 | 11.08 | 96.1 | 2.0 | 0.8 | — | 1.2 | 23 | 63.6 | 12.5 |
| | Ph 3.5 6 h | 100 | 10.85 | 97.2 | 2.1 | 0.7 | — | — | 26.9 | 59.1 | 12.5 |

### Example 2. Antibody Drug Conjugates

After Protein A purification, bispecific antibodies T-6F7-E-6C4 and T-6F7-E-1G11 were dialyzed and concentrated in PBS buffer by ultrafiltration. The concentration was determined by UV absorption. These antibodies were used for the subsequent antibody drug coupling reactions.

Coupling of Antibodies with Drug Molecules

The purified antibodies were coupled with MMAE (monomethyl auristatin E) or MMAF (monomethyl auristatin F) through a maleimidocaproyl-valine-citrulline-p-aminobenzyloxycarbonyl (VC) linker.

For the names of antibody-drug conjugates, “ADC” is added directly after the antibody name when the antibody is coupled to MMAE. For example, if T-6F7-E-6C4 with IgG1 constant region is coupled to MMAE, it is named as T-6F7-E-6C4-ADC. Similarly, if Cetuximab analog with IgG1 constant region is coupled to MMAE, it is named as Cetuximab analog-ADC. If the antibody portion analog of MRG003 with IgG1 constant region is coupled to MMAE, it is named as MRG003-ADC. “SI” is added to the name if the constant region contains SI mutations, leading to names such as Sacituzumab-SI analog-ADC, DS-1062-SI analog (DXd) and DS-1062-SI analog (MMAE).

HIC-HPLC was used to detect the coupling of antibodies with drug molecules. In the HIC-HPLC experiments, an Agilent 1260 chromatography system (connected with ProPac™ HIC-10 column (4.6×250 mm, Thermo Scientific)) was used, and samples were diluted using mobile phase A to 0.5 mg/mL. The following parameters were used: mobile phase A: 0.9 M ammonium sulfate, 0.1 M phosphate buffer (PB), 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 mL; sample tray temperature: about 6° C.; and running time: 45 minutes.

For isotype control, human IgG1 was coupled to MMAE to form isotype-ADC (ISO-ADC). The HIC-HPLC detection results showed that the drug-to-antibody ratio (DAR) of ADC is about 4.

### In Vitro Killing Activity

Different concentrations of purified antibodies (10 μg/mL, 3.333 μg/mL, 1.111 μg/mL, 0.370 μg/mL, 0.123 μg/mL, 0.041 μg/mL, 0.014 μg/mL, and 0.005 μg/mL) and corresponding ADCs were used to treat human epidermoid carcinoma cell line A431 (ATCC, Cat #: CRL-1555), human breast cancer cell line MCF-7, human lung cancer cell line NCI-H226 or NCI-H292 (5×10³) cultured in a cell culture plate, and the killing activity was detected after 3 days of incubation in IncuCyte (Sartorius AG, IncuCyte® S3). The results are shown in the table below.

DS-1062 (Datopotamab deruxtecan) is a TROP2-targeting antibody drug conjugate containing the drug deruxtecan (DXd). SI mutations (EU numbering: S239D and I332E mutations) were introduced in the constant region of DS-1062, resulting in a DS-1062-SI analog (DXd). We further replaced the drug DXd with MMAE, resulting in a DS-1062-SI analog (MMAE). The heavy chain and light chain sequences of the DS-1062-SI analog (DXd or MMAE) are shown as SEQ ID NO: 33 and SEQ ID NO: 34, respectively.

TABLE 5

| | IC50 (μg/mL) | | | |
|---|---|---|---|---|
| | | | NCI- | NCI- |
| ADCs | A431 | MCF-7 | H292 | H226 |
| ISO-ADC | 1.7630 | NA | 4.0820 | NA |
| DS-1062-SI analog (DXd) | 0.7576 | NA | — | NA |
| T-6F7-E-1G11-ADC | 0.0369 | 0.3117 | 0.0974 | 1.2820 |
| T-6F7-E-6C4-ADC | 0.0022 | 1.0060 | 0.0728 | 0.3130 |

(“NA” means no in vitro killing activity; “—” means not tested)

The above results showed that T-6F7-E-1G11-ADC and T-6F7-E-6C4-ADC had good in vitro killing activity.

In another experiment, different concentrations of antibody or ADCs (10 μg/mL, 3.333 μg/mL, 1.111 μg/mL, 0.370 μg/mL, 0.123 μg/mL, 0.041 μg/mL, 0.014 μg/mL, 0.004 μg/mL and 0.0015 μg/mL) were used to treat pancreatic cancer Pan.02.03 cells (TROP2⁺EGFR8⁺), human pancreas adenocarcinoma BxPC-3 cells (TROP2⁺EGFR⁺), human lung cancer NCI-H292 cells (TROP2⁺EGFR⁺) cultured in a cell culture plate, and the killing activity was detected after 72 hours of incubation using PrestoBlue™ Cell Viability Reagent. The results are shown in the table below.

Sacituzumab govitecan, from Immunomedics, Inc, is a humanized anti-TROP2 monoclonal antibody-drug conjugate.

TABLE 6

| | | IC50 (μg/mL) | | |
|---|---|---|---|---|
| Groups | ADCs | Panc 02.03 | BxPC-3 | NCI-H292 |
| G1 | ISO-ADC | 7.9810 | 3.4390 | 3.2020 |
| G2 | Sacituzumab govitecan | 0.2689 | 5.3110 | 0.1910 |
| G3 | T-6F7-SI-ADC | 0.0290 | 0.0332 | 0.0458 |
| G4 | E-6C4-ADC | 0.0139 | 0.0484 | 0.0552 |
| G5 | T-6F7-E-6C4-ADC | 0.0182 | 0.0205 | 0.0368 |

(“NA” means no in vitro killing activity; “—” means not tested)

The results showed that T-6F7-E-6C4-ADC (G5) showed tumor killing efficacy on several cell lines, comparable to its parental TROP2 or EGFR ADCs (G3, G4).

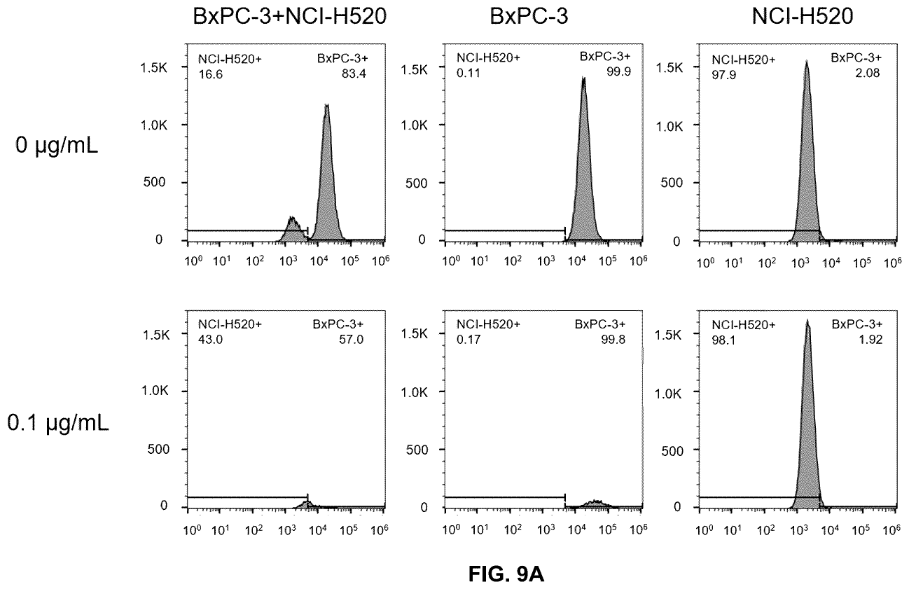

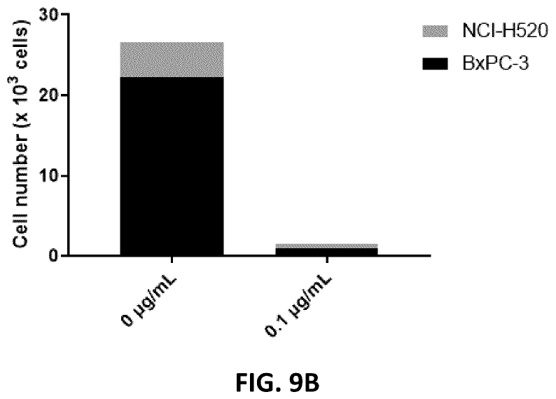

In another experiment, 0.1 μg/mL T-6F7-E-6C4-ADC was added to 3 groups of cells: group 1: BxPC-3 cell+NCI-H520 cell; group 2: BxPC-3 cell; and group 3: NCI-H520 cell (TROP2⁻ EGFR⁻) to test the tumor killing efficacy. In the control group, no T-6F7-E-6C4-ADC was added. After co-incubating for 72 hours at 37° C. in 5% CO₂, cell pellets were stained with dead and viable dye (eBioscience™ Fixable Viability Dye eFluor™ 780, eBioscience, Cat #: 65-0865-14) for flow cytometry analysis. The results are shown in FIG. 9A, which showed that T-6F7-E-6C4-ADC exhibited strong tumor killing activity for TROP2-EGFR double positive BxPC-3 cell, but didn't exhibit tumor killing activity for TROP2-EGFR double negative NCI-H520 cell. However, in group 1 when BxPC-3 cells and NCI-H520 cells were co-cultured (FIGS. 9A-9B), T-6F7-E-6C4-ADC exhibited a strong tumor killing activity to NCI-H520 cell, indicating that T-6F7-E-6C4-ADC showed a clear in vitro bystander killing effect.

### Example 3. Anti-Tumor Activity in A431 Xenograft Model

The antibodies or ADCs were tested for their effects on tumor growth in vivo in a model of epidermoid carcinoma. Specifically, about 5×10⁶ A431 cells were injected subcutaneously in B-NDG mice (Biocytogen Pharmaceuticals (Beijing) Co., Ltd., Cat #: B-CM-002). When the tumors in the mice reached a volume of about 300 mm³, the mice were randomly placed into different groups based on tumor volume. The mice were then injected with phosphate buffer saline (PBS), ADCs or antibodies. Details are shown in the table below.

The lengths of the long axis and the short axis of the tumor were measured and the volume of the tumor was calculated as 0.5×(long axis)×(short axis)². The tumor growth inhibition (TGI) was calculated using the following formula: TGI (%)=[1−(Ti−T0)/(Vi−V0)]×100%. Ti is the average tumor volume in the treatment group on day i. T0 is the average tumor volume in the treatment group on day zero. Vi is the average tumor volume in the control group on day i. V0 is the average tumor volume in the control group on day zero. T-test was performed for statistical analysis. A TGI higher than 60% indicates clear suppression of tumor growth. P<0.05 is a threshold to indicate significant difference.

TABLE 7

| | No. of | | | | | Total No. of |
|---|---|---|---|---|---|---|
| Group | mice | Antibodies/ADCs | Dosage | Route | Frequency | administration |
| G1 | 5 | PBS | — | i.v. | [symbol] W | 2 |
| G2 | 5 | ISO-ADC | 3 mg/kg | i.v. | [symbol] W | 2 |
| G3 | 5 | Sacituzumab-SI analog-ADC | 3 mg/kg | i.v. | [symbol] W | 2 |
| G4 | 5 | Cetuximab analog-ADC | 3 mg/kg | i.v. | [symbol] W | 2 |
| G5 | 5 | T-6F7-E-1G11-ADC | 3 mg/kg | i.v. | [symbol] W | 2 |
| G6 | 5 | T-6F7-E-6C4-ADC | 3 mg/kg | i.v. | [symbol] W | 2 |
| G7 | 5 | DS-1062-SI analog (DXd) | 3 mg/kg | i.v. | [symbol] W | 2 |
| G8 | 5 | T-6F7-E-1G11 | 10 mg/kg | i.p. | BIW | 4 |
| G9 | 5 | T-6F7-E-6C4 | 10 mg/kg | i.p. | BIW | 4 |

[symbol]

indicates data missing or illegible when filed

The body weight of the mice was also measured twice a week. On the day of grouping (Day 0), the average body weight of each group was in the range of 20.7 g-22.1 g. At the end of the experiment (Day 21), the average weight of each group was in the range of 20.4 g-22.9 g. Thus, the average weight change of each group was in the range of 98.5%-107.9%. The results showed that the tested antibodies were well tolerated and were not obviously toxic to the mice.

The table below summarizes the results for this experiment, including the tumor volumes on the day of grouping (Day 0), 14 days after the grouping (Day 14) and at the end of the experiment (Day 21); the survival rate of the mice; TGI (%); and the statistical differences (P value) of body weight and tumor volume between the treatment and control groups.

TABLE 8

| | | | | | | P value | |
|---|---|---|---|---|---|---|---|
| | Tumor volume (mm³) | | | | TGI | Body | Tumor |
| Group | Day 0 | Day 14 | Day 21 | Survival | (%) | weight | volume |
| G1 | 298 ± 9 | 1398 ± 187 | 2169 ± 244 | 5/5 | NA | NA | NA |
| G2 | 299 ± 11 | 1402 ± 217 | 2139 ± 340 | 5/5 | 1.6 | 0.633 | 0.944 |
| G3 | 299 ± 15 | 931 ± 225 | 1665 ± 444 | 5/5 | 27.0 | 1.000 | 0.349 |
| G4 | 299 ± 22 | 922 ± 96 | 1775 ± 270 | 5/5 | 21.1 | 0.562 | 0.311 |
| G5 | 299 ± 17 | 394 ± 100 | 715 ± 240 | 5/5 | 77.8 | 0.308 | 0.003 |
| G6 | 299 ± 19 | 262 ± 22 | 299 ± 60 | 5/5 | 100 | 0.698 | 7.3E−05 |
| G7 | 299 ± 8 | 1057 ± 162 | 1795 ± 352 | 5/5 | 20.0 | 0.694 | 0.407 |
| G8 | 298 ± 25 | 626 ± 87 | 933 ± 174 | 5/5 | 66.1 | 0.191 | 0.003 |
| G9 | 299 ± 32 | 541 ± 59 | 721 ± 85 | 5/5 | 77.5 | 0.359 | 0.001 |

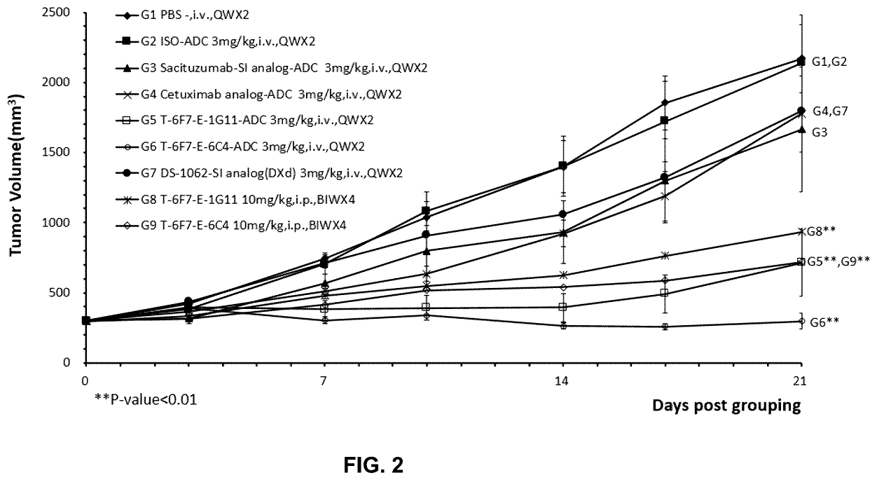

The tumor volume of mice in different groups treated with the antibodies, ADCs, or PBS are shown in FIG. 2. The treatment groups (G3-G9) showed better tumor inhibitory effects compared with the control groups (G1-G2), which were treated with PBS or ISO-ADC.

In addition, the anti-TROP2/EGFR bispecific antibody ADCs (G5-G6) and the anti-TROP2/EGFR bispecific antibodies (G8-G9) showed better tumor inhibitory effects compared with Sacituzumab-SI analog-ADC, Cetuximab analog-ADC or DS-1062-SI analog (DXd).

### Example 4. Anti-Tumor Activity in Panc 02.03 Xenograft Model

The ADCs were tested for their effects on tumor growth in vivo in a xenograft model of pancreatic adenocarcinoma. Specifically, about 2×10⁶ pancreatic adenocarcinoma epithelial Panc 02.03 (ATCC, Cat #: CRL-2553) cells were injected subcutaneously in B-NDG mice. When the tumors in the mice reached a volume of about 200 mm³, the mice were randomly placed into different groups based on tumor volume. The mice were then injected with PBS or ADCs by intravenous (i.v.) administration. The frequency of administration was once a week (1 administrations in total). Details are shown in the table below.

TABLE 9

| | No. of | | | | | Total No. of |
|---|---|---|---|---|---|---|
| Group | mice | ADCs | Dosage | Route | Frequency | administration |
| G1 | 5 | PBS | — | i.v. | [symbol] W | 1 |
| G2 | 5 | ISO-ADC | 10 mg/kg | i.v. | [symbol] W | 1 |
| G3 | 5 | DS-1062-SI analog (MMAE) | 10 mg/kg | i.v. | [symbol] W | 1 |
| G4 | 5 | Cetuximab analog-ADC | 10 mg/kg | i.v. | [symbol] W | 1 |
| G5 | 5 | T-6F7-E-1G11-ADC | 3 mg/kg | i.v. | [symbol] W | 1 |
| G6 | 5 | T-6F7-E-1G11-ADC | 10 mg/kg | i.v. | [symbol] W | 1 |
| G7 | 5 | T-6F7-E-6C4-ADC | 3 mg/kg | i.v. | [symbol] W | 1 |
| G8 | 5 | T-6F7-E-6C4-ADC | 10 mg/kg | i.v. | [symbol] W | 1 |

[symbol]

indicates data missing or illegible when filed

During the experimental period, little difference was observed in the body weight of mice across the groups.

The table below summarizes the results for this experiment, including the tumor volumes on the day of grouping (Day 0), 23 days after the grouping (Day 23) and at the end of the experiment (Day 40); the survival rate of the mice; TGI (%); and the statistical differences (P value) of body weight and tumor volume between the treatment and control groups.

TABLE 10

| | | | | | | P value | |
|---|---|---|---|---|---|---|---|
| | Tumor volume (mm³) | | | | TGI | Body | Tumor |
| Group | Day 0 | Day 23 | Day 40 | Survival | (%) | weight | volume |
| G1 | 192 ± 7 | 1209 ± 67 | 2747 ± 202 | 5/5 | NA | NA | NA |
| G2 | 192 ± 12 | 569 ± 40 | 1664 ± 165 | 5/5 | 42.4 | 0.600 | 0.003 |
| G3 | 192 ± 8 | 396 ± 60 | 1339 ± 208 | 5/5 | 55.1 | 0.827 | 0.001 |
| G4 | 192 ± 11 | 247 ± 13 | 832 ± 33 | 5/5 | 75.0 | 0.983 | 1.4E−05 |
| G5 | 192 ± 8 | 674 ± 120 | 2058 ± 298 | 5/5 | 27.0 | 0.683 | 0.092 |
| G6 | 192 ± 12 | 194 ± 15 | 574 ± 69 | 5/5 | 85.1 | 0.494 | 3.7E−05 |
| G7 | 192 ± 11 | 623 ± 72 | 1809 ± 313 | 5/5 | 36.7 | 0.328 | 0.036 |
| G8 | 192 ± 11 | 225 ± 15 | 643 ± 45 | 5/5 | 82.3 | 0.736 | 7.4E−06 |

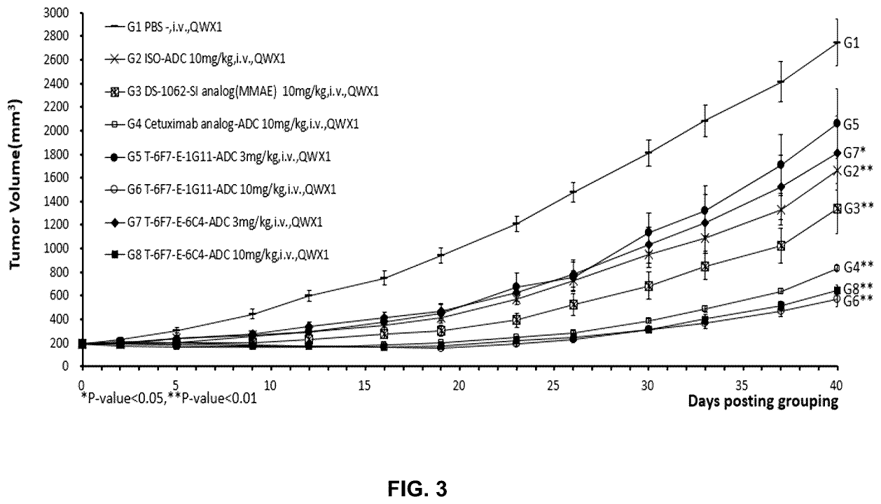

The tumor size in groups treated with the ADCs are shown in FIG. 3. The treatment groups showed different tumor inhibitory effects. Overall, the anti-TROP2/EGFR bispecific antibody ADCs (T-6F7-E-1G11-ADC and T-6F7-E-6C4-ADC) showed better anti-tumor activities compared with the controls (ISO-ADC, Cetuximab analog-ADC and DS-1062-SI analog (MMAE) at a dose of 10 mg/kg. The Anti-TROP2/EGFR Bispecific Antibody ADCs (T-6F7-E-1G11-ADC and T-6F7-E-6C4-ADC) showed dose-dependent antitumor activities.

### Example 5. Anti-Tumor Activity in Pancreatic Adenocarcinoma PDX Model

The ADCs were tested for their effects on tumor growth in vivo in a xenograft model of pancreatic adenocarcinoma. Specifically, tumor fragments derived from patients with Pancreatic Adenocarcinoma were inoculated subcutaneously in B-NDG mice. When the tumors in the mice reached a volume of about 250-300 mm³, the mice were randomly placed into different groups based on tumor volume. The mice were then injected with PBS or ADCs by intravenous (i.v.) administration. Details are shown in the table below.

TABLE 11

| | No. of | | | | | Total No. of |
|---|---|---|---|---|---|---|
| Group | mice | ADCs | Dosage | Route | Frequency | administration |
| G1 | 5 | PBS | — | i.v. | [symbol] W | 2 |
| G2 | 5 | ISO-ADC | 3 mg/kg | i.v. | [symbol] W | 2 |
| G3 | 5 | Cetuximab analog-ADC | 3 mg/kg | i.v. | [symbol] W | 2 |
| G4 | 5 | Sacituzumab govitecan | 3 mg/kg | i.v. | [symbol] W | 2 |
| G5 | 5 | T-6F7-E-1G11-ADC | 3 mg/kg | i.v. | [symbol] W | 2 |
| G6 | 5 | T-6F7-E-6C4-ADC | 3 mg/kg | i.v. | [symbol] W | 2 |
| G7 | 5 | T-6F7-SI-ADC | 3 mg/kg | i.v. | [symbol] W | 2 |

[symbol]

indicates data missing or illegible when filed

During the experimental period, almost no difference was observed in the body weight of mice across the groups.

The table below summarizes the results for this experiment, including the tumor volumes on the day of grouping (Day 0), 31 days after the grouping (Day 31) and at the end of the experiment (Day 41); the survival rate of the mice; TGI (%); and the statistical differences (P value) of tumor volumes between the treatment and control groups.

TABLE 12

| | Tumor volume (mm³) | | | | TGI | P |
|---|---|---|---|---|---|---|
| Group | Day 0 | Day 31 | Day 41 | Survival | (%) | value |
| G1 | 252 ± 14 | 2272 ± 231 | 2555 ± 262 | 3/6 | NA | NA |
| G2 | 252 ± 17 | 1529 ± 145 | 2375 ± 265 | 6/6 | 7.8 | 0.685 |
| G3 | 252 ± 18 | 192 ± 17 | 697 ± 61 | 6/6 | 80.7 | 2.8E−05 |
| G4 | 252 ± 16 | 2084 ± 302 | 2214 ± 464 | 3/6 | 14.8 | 0.557 |
| G5 | 253 ± 17 | 173 ± 80 | 422 ± 72 | 6/6 | 92.7 | 1.5E−05 |
| G6 | 253 ± 15 | 78 ± 27 | 268 ± 83 | 6/6 | 99.3 | 1.2E−05 |
| G7 | 252 ± 15 | 290 ± 57 | 675 ± 92 | 6/6 | 81.7 | 5.7E−05 |

The tumor volumes in all treatment groups (G3-G7) were smaller than those in the control group (G1 and G2). The treatment groups had different tumor inhibitory effects. The TROP2/EGFR bispecific antibody ADCs at a dose level of 3 mg/kg (G5-G6) showed sustained and potent tumor suppression effects. T-6F7-E-6C4-ADC (G6) had the highest TGI of 99.3%. The TGI values of all tested TROP2/EGFR bispecific antibody ADCs (G5-G6) were higher than that of the control (Cetuximab analog-ADC or Sacituzumab govitecan).

### Example 6. Anti-Tumor Activity in Patient-Derived Lung Cancer Xenograft Model

The ADCs were tested for their effects on tumor growth in vivo in a xenograft model of lung cancer. Specifically, tumor fragments derived from patients with lung cancer were inoculated subcutaneously in B-NDG mice. When the tumors in the mice reached a volume of about 250-300 mm³, the mice were randomly placed into different groups based on tumor volume. The mice were then injected with PBS, ADCs or antibodies by intravenous (i.v.) administration. The frequency of administration was once a week (2 administrations in total). Details are shown in the table below.

TABLE 13

| | No. of | | | | | Total No. of |
|---|---|---|---|---|---|---|
| Group | mice | ADCs | Dosage | Route | Frequency | administration |
| G1 | 5 | PBS | — | i.v. | [symbol] W | 2 |
| G2 | 5 | ISO-ADC | 3 mg/kg | i.v. | [symbol] W | 2 |
| G3 | 5 | Sacituzumab govitecan | 3 mg/kg | i.v. | [symbol] W | 2 |
| G4 | 5 | Cetuximab analog-ADC | 3 mg/kg | i.v. | [symbol] W | 2 |
| G5 | 5 | T-6F7-E-6C4-ADC | 3 mg/kg | i.v. | [symbol] W | 2 |

[symbol]

indicates data missing or illegible when filed

During the experimental period, almost no difference was observed in the body weight of mice across the groups.

The table below summarizes the results for this experiment, including the tumor volumes on the day of grouping (Day 0), 13 days after the grouping (Day 13) and at the end of the experiment (Day 20); the survival rate of the mice; TGI (%); and the statistical differences (P value) of tumor volumes between the treatment and control groups.

TABLE 14

| | | | | | | P value | |
|---|---|---|---|---|---|---|---|
| | Tumor volume (mm³) | | | | TGI | Body | Tumor |
| Group | Day 0 | Day 13 | Day 20 | Survival | (%) | weight | volume |
| G1 | 301 ± 17 | 1146 ± 95 | 1624 ± 222 | 4/5 | NA | NA | NA |
| G2 | 301 ± 23 | 750 ± 117 | 1542 ± 111 | 5/5 | 6.2 | 0.694 | 0.737 |
| G3 | 301 ± 24 | 857 ± 131 | 1553 ± 262 | 5/5 | 5.4 | 0.223 | 0.848 |
| G4 | 301 ± 23 | 898 ± 173 | 1380 ± 190 | 5/5 | 18.4 | 0.808 | 0.430 |
| G5 | 301 ± 25 | 400 ± 42 | 801 ± 83 | 5/5 | 62.2 | 0.372 | 0.007 |

The treatment groups had different tumor inhibitory effects. The TGI value of TROP2/EGFR bispecific antibody ADC at a dose level of 3 mg/kg (G5) was higher than those of the positive control ISO-ADC, Cetuximab analog-ADC and Sacituzumab govitecan.

### Example 7. Anti-Tumor Activity in NCI-H1292 Xenograft Model

The antibodies or ADCs were tested for their effects on tumor growth in vivo in a xenograft model of lung cancer. Specifically, about 5×10⁶ NCI-H292 cells were injected subcutaneously in B-NDG mice. When the tumors in the mice reached a volume of about 200 mm³, the mice were randomly placed into different groups based on tumor volume. The mice were then injected with PBS, antibodies, or ADCs by intravenous (i.v.) administration. Details are shown in the table below.

Cetuximab is an EGFR-targeting chimeric monoclonal IgG1 antibody from Merck.

TABLE 15

| | | | | | | | Total |
|---|---|---|---|---|---|---|---|
| | No. of | Antibodies/ | | | | | No. of |
| Group | mice | ADCs | Dosage | | Route | Frequency | ad [symbol] inistration |
| G1 | 6 | PBS | — | | i.v. | [symbol] | 2 |
| G2 | 6 | ISO-ADC | 10 | mg/kg | i.v. | [symbol] | 2 |
| G3 | 6 | Cetuximab | 10 | mg/kg | i.v. | [symbol] | 4 |
| G4 | 6 | MRG003-ADC | 10 | mg/kg | i.v. | [symbol] | 2 |
| G5 | 6 | T-6F7-E-6C4-ADC | 1 | mg/kg | i.v. | [symbol] | 2 |
| G6 | 6 | T-6F7-E-6C4-ADC | 3 | mg/kg | i.v. | [symbol] | 2 |
| G7 | 6 | T-6F7-E-6C4-ADC | 10 | mg/kg | i.v. | [symbol] | 2 |
| G8 | 6 | T-6F7-SI-ADC | 3 | mg/kg | i.v. | [symbol] | 2 |
| G9 | 6 | E-6C4-ADC | 3 | mg/kg | i.v. | [symbol] | 2 |

[symbol]

indicates data missing or illegible when filed

MRG003 is an antibody-drug conjugate consisting of fully human IgG1 monoclonal antibody targeting EGFR conjugated to monomethyl auristatin E (MMAE) for the treatment of solid tumors, which is in early clinical development at Shanghai Miracogen. The heavy chain and light chain sequences of antibody of MRG003 are shown in SEQ ID NO: 35 and SEQ ID NO: 36, respectively.

The table below summarizes the results for this experiment, including the tumor volumes on the day of grouping (Day 0), 18 days after grouping (Day 18) and at the end of the experiment (Day 35); the survival rate of the mice; TGI (%); and the statistical differences (P value) of tumor volume between the treatment and control groups.

TABLE 16

| | | | | | | | P value |
|---|---|---|---|---|---|---|---|
| | Tumor volume (mm³ ) | | | Survival | Tumor- | TGI | (Tumor |
| Group | Day 0 | Day 18 | Day 35 | Day 35 | free | (%) | volume) |
| G1 | 199 ± 7 | 1105 ± 15 | 1930 ± 183 | 3/6 | 0 | NA | NA |
| G2 | 199 ± 11 | 720 ± 162 | 1680 ± 383 | 5/6 | 0 | 14.4 | 0.653 |
| G3 | 199 ± 10 | 26 ± 11 | 352 ± 79 | 6/6 | 1 | 91.1 | 3.07E−05 |
| G4 | 199 ± 9 | 0 | 208 ± 40 | 6/6 | 0 | 99.5 | 3.82E−06 |
| G5 | 199 ± 10 | 495 ± 39 | 1115 ± 93 | 4/6 | 0 | 47.1 | 0.008 |
| G6 | 199 ± 11 | 156 ± 27 | 575 ± 53 | 6/6 | 0 | 78.3 | 3.01E−05 |
| G7 | 199 ± 9 | 0 | 0 | 6/6 | 6 | 111.5 | 8.52E−07 |
| G8 | 198 ± 9 | 344 ± 17 | 842 ± 82 | 6/6 | 0 | 62.8 | 3.56E−04 |
| G9 | 199 ± 7 | 277 ± 19 | 844 ± 50 | 6/6 | 0 | 62.7 | 1.11E−04 |

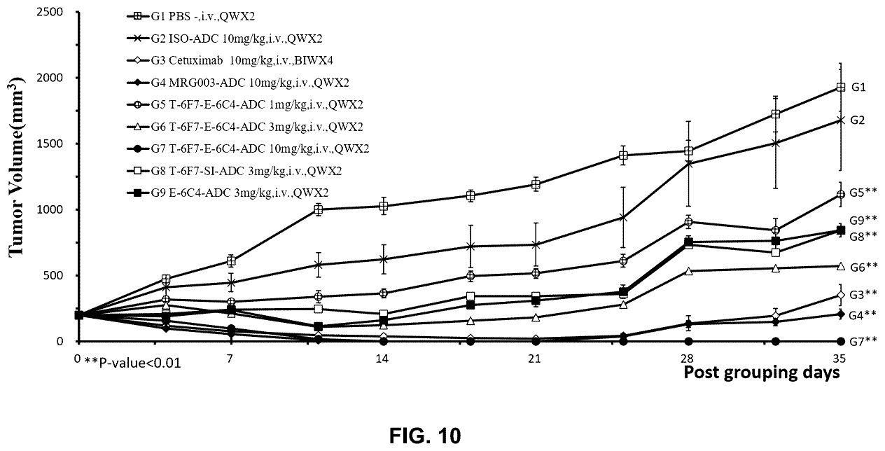

The tumor size in groups treated with the antibodies or ADCs are shown in FIG. 10. The anti-TROP2/EGFR bispecific antibody ADC T-6F7-E-6C4-ADC (G5-G7) showed a better anti-tumor activity compared with the controls (Cetuximab and MRG003-ADC) at a dose level of 10 mg/kg, and obtained a better tumor inhibitory effect than the corresponding parental ADCs (T-6F7-SI-ADC and E-6C4-ADC) at a dose level of 3 mg/kg. In addition, T-6F7-E-6C4-ADC showed a dose-dependent antitumor activity. Specifically, T-6F7-E-6C4-ADC (G7) showed a sustained anti-tumor activity in all 6 mice, which were tumor-free on Day 35 post grouping.

### Example 8. Anti-Tumor Activity in NUGC-4 Xenograft Model

The antibodies or ADCs were tested for their effects on tumor growth in vivo in a xenograft model of gastric cancer. Specifically, about 5×10⁶ NUGC-4 cells were injected subcutaneously in B-NDG mice. When the tumors in the mice reached a volume of about 200 mm³, the mice were randomly placed into different groups based on tumor volume. The mice were then injected with PBS, antibodies, or ADCs by intravenous (i.v.) administration. Details are shown in the table below.

TABLE 17

| | | | | | | | Total |
|---|---|---|---|---|---|---|---|
| | No. of | Antibodies/ | | | | | No. of |
| Group | mice | ADCs | Dosage | | Route | Frequency | ad [symbol] inistration |
| G1 | 6 | PBS | — | | i.v. | [symbol] | 2 |
| G2 | 6 | ISO-ADC | 3 | mg/kg | i.v. | [symbol] | 2 |
| G3 | 6 | Sacituzumab govitecan | 10 | mg/kg | i.v. | BI [symbol] | 4 |
| G4 | 6 | Cetuximab | 10 | mg/kg | i.v. | BI [symbol] | 4 |
| G5 | 6 | MRG003-ADC | 1 | mg/kg | i.v. | [symbol] | 2 |
| G6 | 6 | MRG003-ADC | 3 | mg/kg | i.v. | [symbol] | 2 |
| G7 | 6 | T-6F7-E-6C4-ADC | 1 | mg/kg | i.v. | [symbol] | 2 |
| G8 | 6 | T-6F7-E-6C4-ADC | 3 | mg/kg | i.v. | [symbol] | 2 |
| G9 | 6 | T-6F7-E-6C4-ADC | 10 | mg/kg | i.v. | [symbol] | 2 |

[symbol]

indicates data missing or illegible when filed

The table below summarizes the results for this experiment, including the tumor volumes on the day of grouping (Day 0), 13 days after grouping (Day 13) and at the end of the experiment (Day 27); TGI (%); and the statistical differences (P value) of tumor volume between the treatment and control groups.

TABLE 18

| | | | | | P value |
|---|---|---|---|---|---|
| | Tumor volume (mm³) | | | TGI | (Tumor |
| Group | Day 0 | Day 13 | Day 27 | (%) | volume) |
| G1 | 193 ± 12 | 868 ± 57 | 1985 ± 183 | NA | NA |
| G2 | 193 ± 14 | 575 ± 65 | 1737 ± 149 | 13.9 | 0.316 |
| G3 | 192 ± 19 | 521 ± 57 | 1566 ± 143 | 23.4 | 0.101 |
| G4 | 193 ± 12 | 807 ± 70 | 2323 ± 250 | −18.8 | 0.301 |
| G5 | 194 ± 17 | 844 ± 143 | 2122 ± 264 | −7.6 | 0.679 |
| G6 | 193 ± 10 | 338 ± 54 | 1328 ± 139 | 36.7 | 0.017 |
| G7 | 193 ± 18 | 610 ± 68 | 1482 ± 163 | 28.1 | 0.067 |
| G8 | 193 ± 14 | 171 ± 48 | 680 ± 169 | 72.9 | 3.77E−04 |
| G9 | 193 ± 11 | 27 ± 6 | 11 ± 3 | 110.2 | 7.79E−07 |

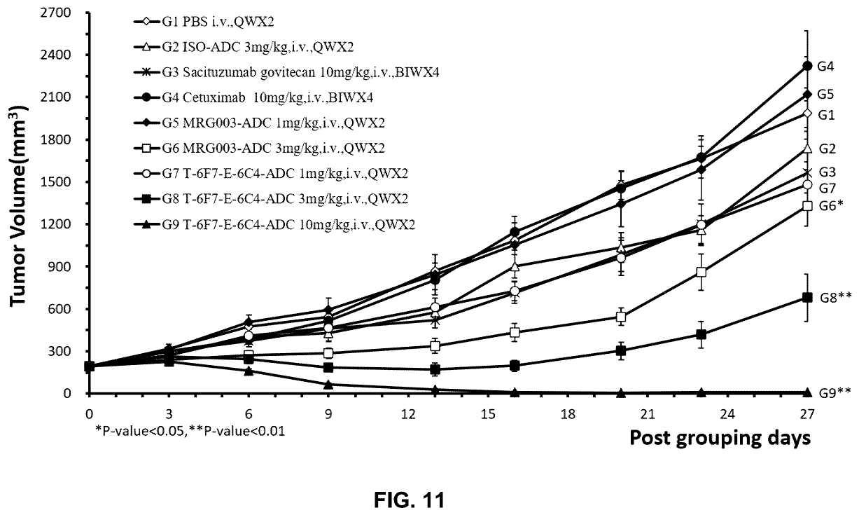

The tumor size in groups treated with the antibodies or ADCs are shown in FIG. 11. ADC T-6F7-E-6C4-ADC (G7, G8, G9) showed a better anti-tumor activity compared with the controls Cetuximab (G4), Sacituzumab govitecan (G3), and MRG003-ADC (G5, G6). In addition, T-6F7-E-6C4-ADC showed a dose-dependent antitumor activity.

### Example 9. Pharmacokinetic Profiles and Plasma Stability

The pharmacokinetic clearance rates of the anti-EGFR/TROP2 bispecific antibody ADC were determined in B-hFcRn mice (Biocytogen Pharmaceuticals (Beijing) Co., Ltd., Cat #: 110001). Specifically, the mice were placed into four groups (6 mice per group), and administered with ISO-ADC (G1, 3 mg/kg; G2, 10 mg/kg) or T-6F7-E-6C4-ADC (G3, 3 mg/kg; G4, 10 mg/kg) by intravenous injection. Blood samples were collected before administration and 15 minutes, 1 day, 3 days, 7 days, 10 days, 14 days and 21 days after administration.

The serum levels of total antibody and ADC were determined by sandwich ELISA. Briefly, Goat Anti-Human IgG (H+L) (Jackson ImmunoResearch Inc., Cat #: 109-005-088) or anti-MMAE mIgG (ACRO Biosystems Inc., Cat #: MME-M5252) was diluted to a final concentration of 2000 ng/mL, added to a 96-well plate (ELISA plate) at 100 μL/well, and then incubated overnight at 2-8° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of blocking buffer (2% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μL/well to each well of the plate, and incubated at 37° C. for 1 hour for determining the serum concentration of total antibody. Alternatively, G-h-IgG κ L-HRP (Abcam, Cat #: ab202549) was added for determining the serum concentration of ADCs. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. Luminescent signals of the plate were measured at 450 nm and 630 nm to calculate the concentrations. The absorbance value and corresponding concentration of the calibration sample prepared by each test product was used to create a standard curve with four parameters (i.e., T_{1/2}, Cₘₐₓ, AUC_{0-21 day}, and CL). The standard curve was used to calculate the total antibody or ADC concentration of each serum sample. A drug concentration-time curve was created using the calculated sample concentration at each time point. Phoenix™ WinNolin 8.3 was used to calculate the pharmacokinetic parameters.

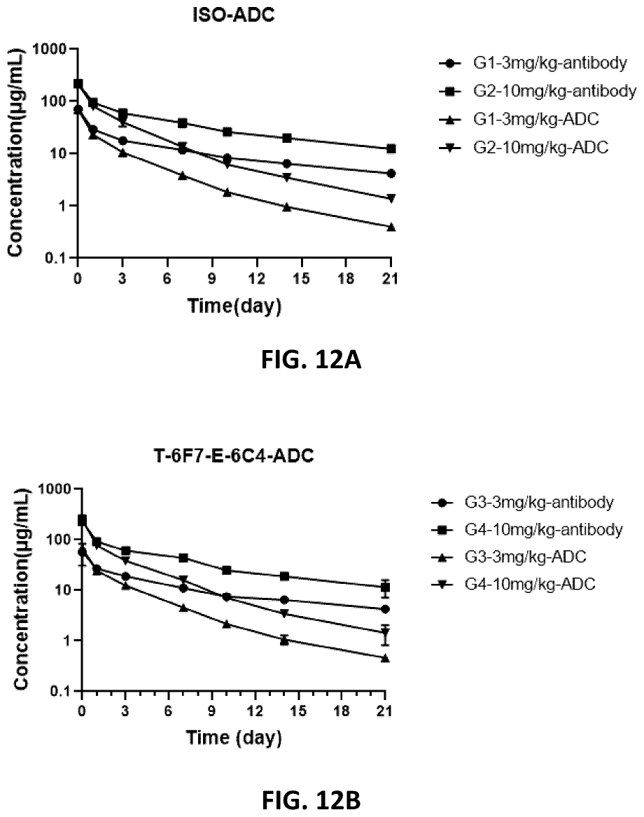

The results are shown in the table below and FIGS. 12A-12B, which showed that T-6F7-E-6C4-ADC exhibited a similar half-life to the isotype control.

TABLE 19

| | | Dose | | T_{1/2} | Cₘₐₓ | AUC_{0-21 day} | CL |
|---|---|---|---|---|---|---|---|
| Group | ADCs | (mg/kg) | LBA | (day) | (μg/mL) | (day*μg/mL) | (mL/day/kg) |
| G1 | ISO-ADC | 3 | ADC | 5.069 | 70.435 | 128.429 | 22.86 |
| | | | Antibody | 11.365 | 71.15 | 253.356 | 9.333 |
| G2 | ISO-ADC | 10 | ADC | 5.088 | 218.129 | 444.176 | 22.089 |
| | | | Antibody | 10.335 | 221.353 | 812.886 | 10.042 |
| G3 | T-6F7-E- | 3 | ADC | 5.053 | 69.081 | 139.525 | 21.051 |
| | 6C4-ADC | | Antibody | 13.318 | 67.243 | 246.488 | 9.182 |
| G4 | T-6F7-E- | 10 | ADC | 4.818 | 252.129 | 461.152 | 21.28 |
| | 6C4-ADC | | Antibody | 10.581 | 230.834 | 821.148 | 10.032 |

T_{1/2}: Terminal Half Life ;
Cₘₐₓ: Max Concentration ;
AUC_{0-21 day}: Area under Blood Concentration-time Curve 0-21 day
CL: Clearance

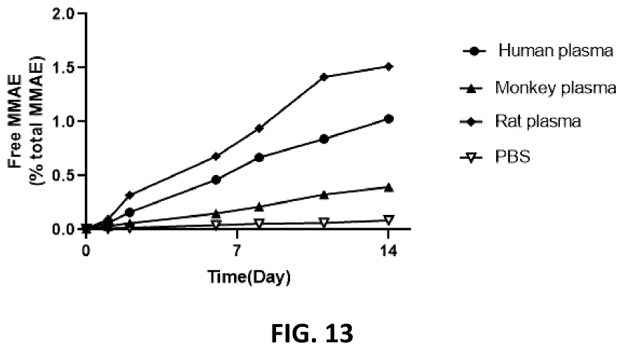

In another experiment, plasma stability of T-6F7-E-6C4-ADC was determined in human plasma, monkey (Macaca fascicularis) plasma, and rat (SD rat) plasma. Specifically, T-6F7-E-6C4-ADC was added to plasma of human, Macaca fascicularis, and SD rat, respectively, to a terminal concentration of 100 μg/mL. In the control group, the plasma was replaced by PBS with 0.5% BSA. The contents of free MMAE and ADC were determined in 0 day, 1 day, 2 days, 6 days, 8 days, 11 days and 14 days after adding the T-6F7-E-6C4-ADC and the ratios of free MMAE and ADC were calculated. The results are shown in FIG. 13.

The results showed that the percentages of free MMAE to the total MMAE in human plasma, monkey plasma and rat plasma were all less than 2% after 14 days, indicating that T-6F7-E-6C4-ADC was relatively stable in human, monkey and rat plasma.

### Example 10. Antibody Drug Conjugates

Coupling of Antibodies with Drug Molecules

The purified antibodies were coupled with CPT-1, CPT-2, CPT-3, or CPT-4 through a CPT-L linker. For the names of antibody-drug conjugates, CPTx (x=1, 2, 3, or 4) is added directly after the antibody name. For example, when T-6F7-E-6C4 is coupled to CPT-1, it is named as T-6F7-E-6C4-CPT1. As another example, when T-6F7-E-6C4 is coupled to CPT-2, it is named as T-6F7-E-6C4-CPT2. Exemplary ADCs obtained by this method included: T-6F7-E-6C4-CPT1 and T-6F7-E-6C4-CPT2.

MS (Mass Spectrometry) was used to detect the coupling of antibodies with drug molecules. A human IgG1 molecule was coupled to CPT-2 to form isotype-CPT2 (ISO-CPT2), as an isotype control. The MS detection results showed that the drug-to-antibody ratio (DAR) of the ADCs was about 4 or 8. With regard to the ADC names, if the DAR of T-6F7-E-6C4-CPT2 is about 4, the ADC is named T-6F7-E-6C4-CPT2 (DAR4). If the DAR of T-6F7-E-6C4-CPT2 is about 8, the ADC is named T-6F7-E-6C4-CPT2 (DAR8).

Preparation Linker-Therapeutic agent (II′)-2 below (CPT-2 linked with CPT-L):

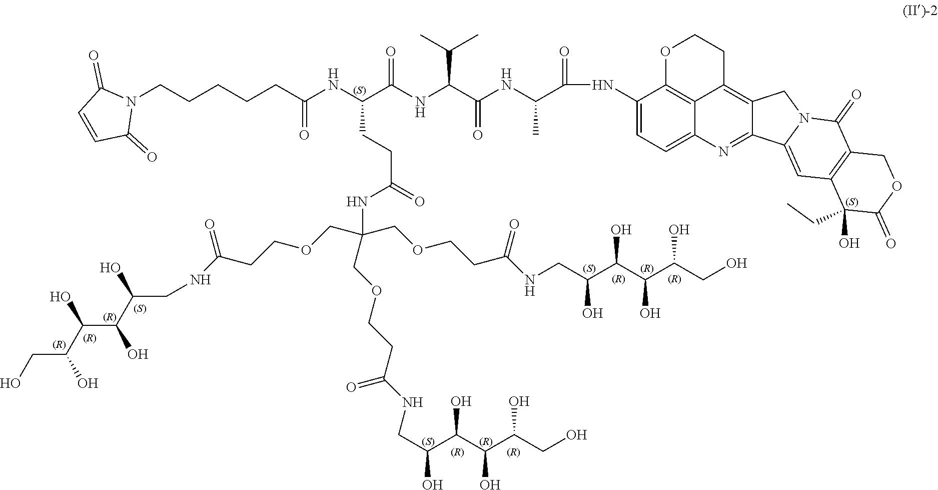

### Synthesis of (S)—N5-((2R,3R,4R,5S,21S,22R,23R,24R)-1,2,3,4,5,21,22,23,24,25-decahydroxy-8,18-dioxo-13-((3-oxo-3-(((2S,3R,4R,5R)-2,3,4,5,6-pentahydroxyhexyl)amino)propoxy)methyl)-11,15-dioxa-7,19-diazapentacosan-13-yl)-2-(6-(2,5-dioxo-2,5-dihydro-1H-pyrrol-1-yl)hexanamido)-N1-((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)pentanediamide (Compound (II′)-2)

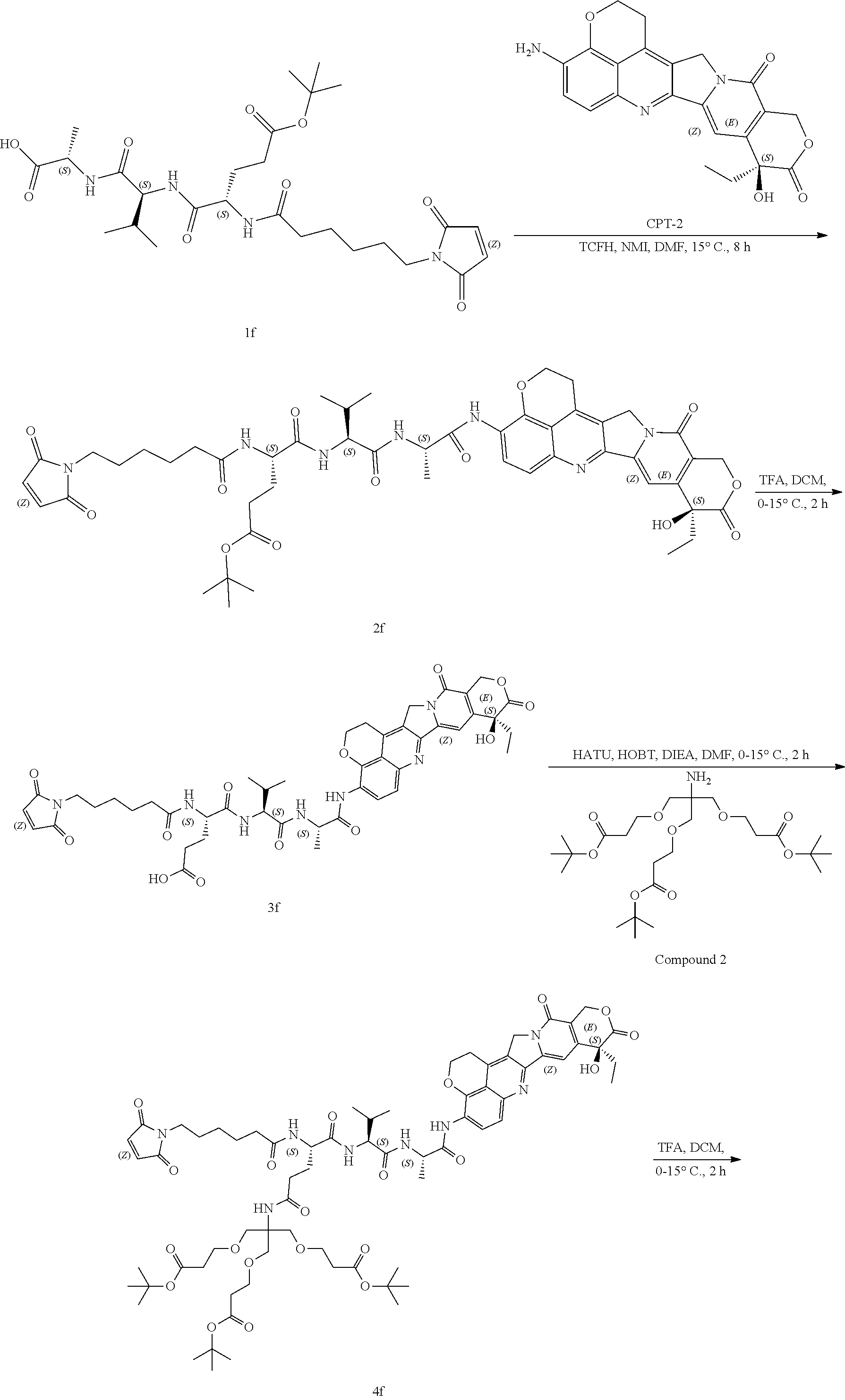

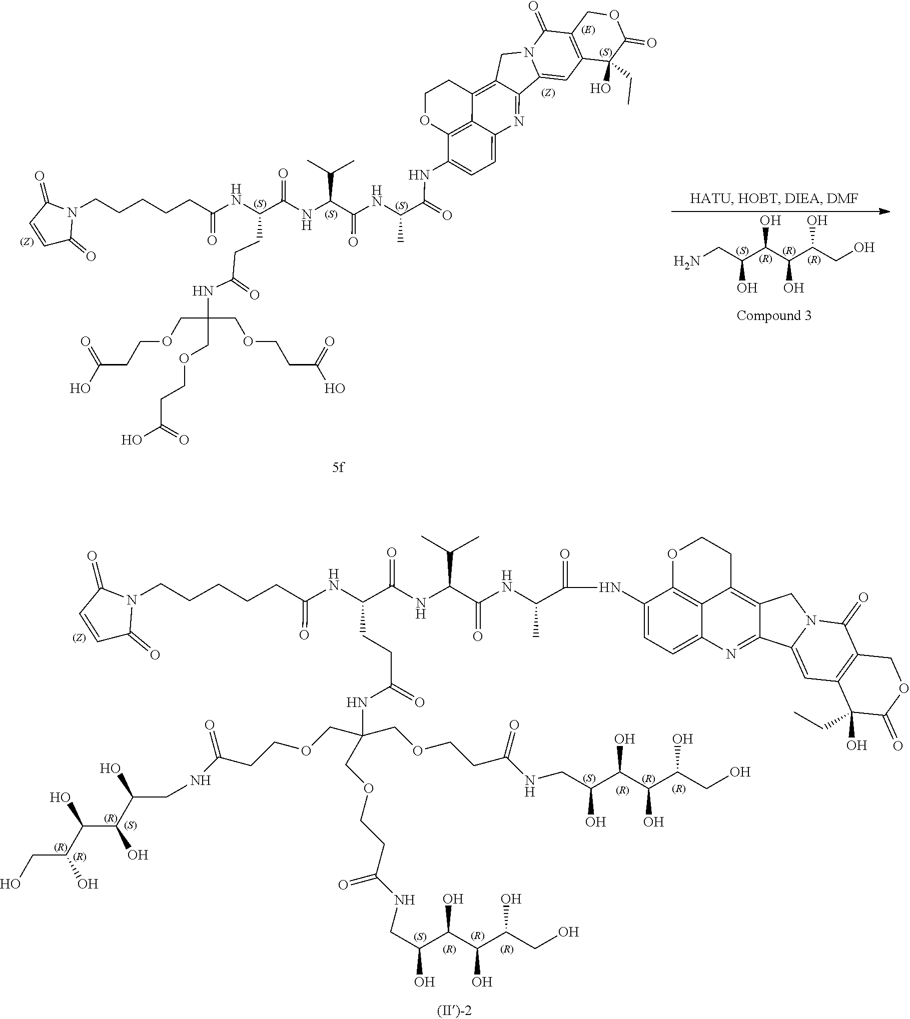

### Step 1: Synthesis of tert-butyl (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoate (Compound 2f)

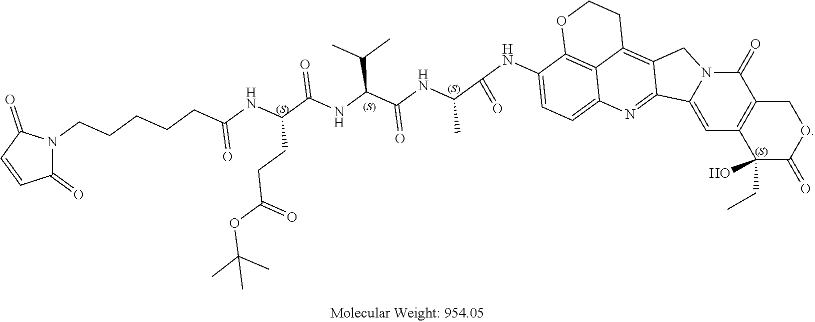

1-methylimidazole (0.043 mL, 0.537 mmol) was added to a mixture of N-[(2S,5S)-12-(2,5-dioxo-2,5-dihydro-1H-pyrrol-1-yl)-5-{3-[(2-methylprop-2-yl)oxy]-3-oxopropyl}-1,4,7-trioxo-2-(prop-2-yl)-3,6-diazadodec-1-yl]-L-alanine (Compound 1f, 166.82 mg, 0.294 mmol) and CPT-2 (115 mg, 0.285 mmol) in DMF (1.9 mL) and then TCFH (86.04 mg, 0.307 mmol) was added to the mixture. Then, the resulting mixture was stirred for 12 hours at 25° C. After the reaction was completed as confirmed by LC-MS, saturated salt water (6 mL) was added to the reaction solution and a large amount of solids were extracted. The resulting mixture was then filtered through a cloth funnel and the solids were washed by water. Afterwards, the residue was dissolved in DCM (30 mL). The organic layers were dried over sodium sulfate, filtered, and the filtrate was concentrated under reduced pressure to obtain the title Compound 2f which was used to next step without purification (155 mg, brown solid, yield: 63.6%).

LC-MS: (ESI) m/z (M+H), 954.5.

### Step 2: Synthesis of (S)-4-(6-(2,5-dioxo-2,5-dihydro-1H-pyrrol-1-yl)hexanamido)-5-(((S)-1-(((S)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-1-oxopropan-2-yl)amino)-3-methyl-1-oxobutan-2-yl)amino)-5-oxopentanoic Acid (Compound 3f)

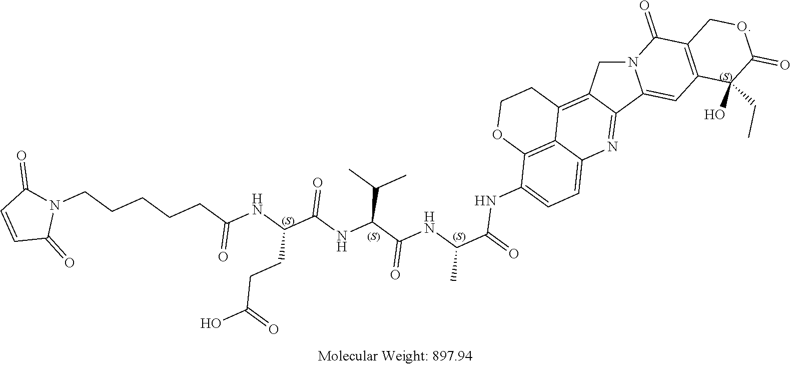

2,2,2-trifluoroacetic acid (0.85 mL, 8.1 mmol) was added to a solution of Compound 2f (155 mg, 0.162 mmol) in DCM (4.25 mL) at 0° C. for 5 minutes and the resulting mixture was naturally warmed up to 15° C. and stirred for 2 hours. After the reaction was completed as confirmed by LC-MS, the mixture was concentrated to yield the title Compound 3f which was used to next step without purification (145.88 mg, yellow solid, quantitative yield).

LC-MS: (ESI) m/z (M+1), 898.6.

### Step 3: Synthesis of tert-butyl (2S,5S,8S)-13,13-bis((3-(tert-butoxy)-3-oxopropoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oate (Compound 4f)

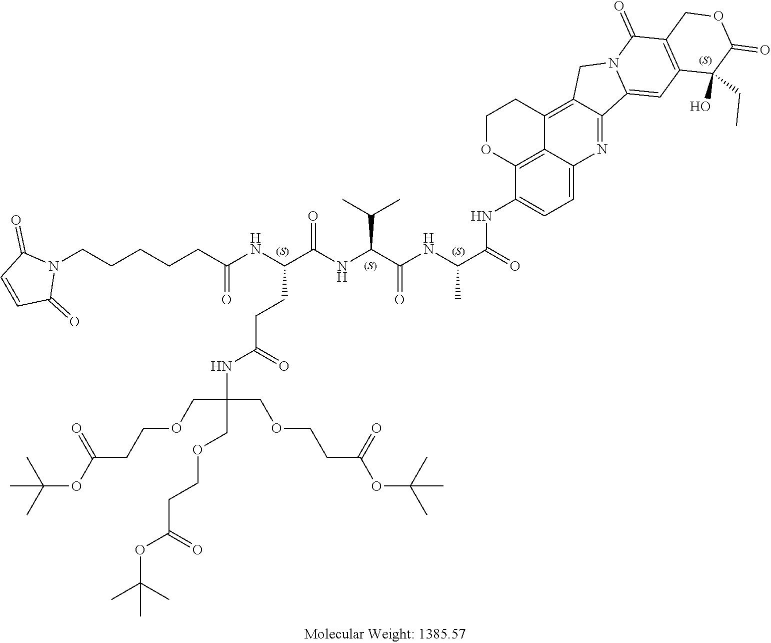

A mixture of Compound 3f (145.88 mg, 0.162 mmol), HATU (74.13 mg, 0.195 mmol), HOBT (26.34 mg, 0.195 mmol) and DIEA (0.081 mL, 0.487 mmol) in DMF (4 mL) was stirred at 0° C. for 5 minutes. 2-methylpropan-2-yl 3-{[9-amino-9-(7, 7-dimethyl-5-oxo-2, 6-dioxaoct-1-yl)-2, 2-dimethyl-4-oxo-3, 7-dioxadec-10-yl]oxy}propanoate (Compound 2, 82.15 mg, 0.162 mmol) was then added to the mixture and the resulting mixture was naturally warmed up to 15° C. and stirred for 1 hour. After the reaction was completed as confirmed by LC-MS, saturated salt water (12 mL) was added to the reaction solution and a large amount of solids were extracted. Then, the mixture was filtered through a cloth funnel and the solids were washed by water followed by the resulting residue being dissolved in DCM (10 mL). Afterwards, the organic layers were dried over sodium sulfate, filtered, and the filtrate was concentrated under reduced pressure to yield the title Compound 4f which was used to next step without purification (188 mg, brown solid, yield: 83.5%).

LC-MS: (ESI) m/z (M+1), 1386.6.

### Step 4: Synthesis of (2S,5S,8S)-13,13-bis((2-carboxyethoxy)methyl)-8-(6-(2,5-dioxo-2,5-dihydro-1H-pyrrol-1-yl)hexanamido)-1-(((S)-9-ethyl-9-hydroxy-10,13-dioxo-1,2,9,10,13,15-hexahydro-12H-pyrano[4,3,2-de]pyrano[3′,4′:6,7]indolizino[1,2-b]quinolin-4-yl)amino)-5-isopropyl-2-methyl-1,4,7,11-tetraoxo-15-oxa-3,6,12-triazaoctadecan-18-oic Acid (Compound 5f)

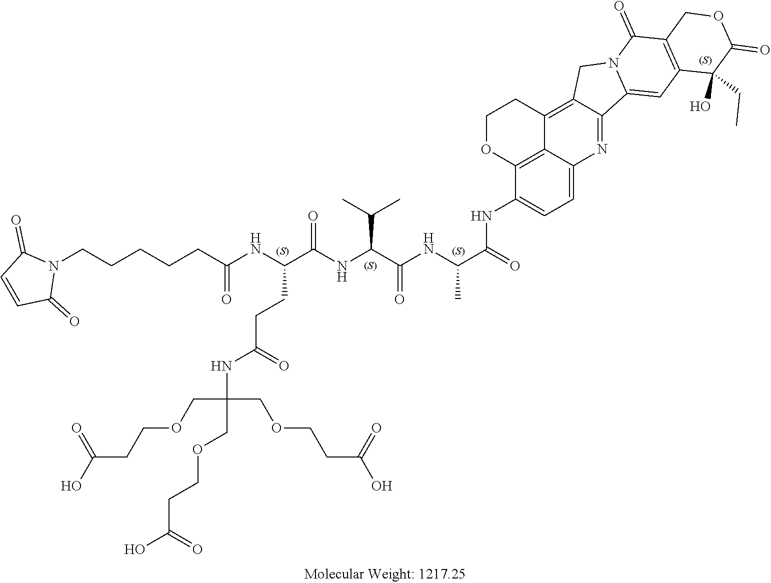

2,2,2-trifluoroacetic acid (0.769 mL, 7.327 mmol) was added to a solution of Compound 4f (188 mg, 0.122 mmol) in DCM (3.18 mL) at 0° C. for 5 minutes. The resulting mixture was then naturally warmed up to 15° C. and stirred for 2 hours. After the reaction was completed as confirmed by LC-MS, the mixture was concentrated to yield Compound 5f which was used to next step without purification (148.65 mg, yellow solid, quantitative yield).

LC-MS: (ESI) m/z (M+1), 1217.6.

### Step 5: Synthesis of Compound (II′)-2

A mixture of Compound 5f (133.785 mg, 0.110 mmol), HATU (158.81 mg, 0.418 mmol), HOBT (56.44 mg, 0.418 mmol) and DIEA (0.145 mL, 0.879 mmol) in DMF (2.8 mL) was stirred at 0° C. for 20 minutes. (2R, 3R, 4R, 5S)-6-aminohexane-1, 2, 3, 4, 5-pentol (Compound 3, 75.67 mg, 0.418 mmol) was then added to the mixture. The resulting mixture was naturally warmed up to 15° C. and stirred for 1 hour before the solvent was removed under vacuum and the residue was purified by Prep-HPLC (TFA) to yield the title compound (II′)-2 (20.0 mg, yellow solid, yield: 12.0%).

LC-MS: (ESI) m/z 1706.8. 1707.8 (M+H).

¹HNMR (400 MHz, DMSO): δ 9.51 (s, 1H), 8.48-8.44 (m, 2H), 8.02 (d, J=7.6 Hz, 1H), 7.78 (t, J=5.2 Hz, 3H), 7.73 (d, J=9.6 Hz, 1H), 7.59 (d, J=8.8 Hz, 1H), 7.32 (s, 1H), 7.13 (s, 1H), 6.99 (s, 2H), 6.49 (s, 1H), 5.44 (s, 2H), 5.28 (s, 2H), 4.64-4.60 (m, 1H), 4.54 (t, J=5.6 Hz, 2H), 4.30-4.20 (m, 4H), 3.61-3.46 (m, 12H), 3.49-3.45 (m, I0H), 3.42-3.36 (m, 17H), 3.33-3.24 (m, 8H), 3.07-2.97 (m, 5H), 2.35-2.32 (m, 6H), 2.13-2.10 (m, 4H), 2.04-2.00 (m, 1H), 1.91-1.84 (m, 3H), 1.71-1.65 (m, 1H), 1.51-1.46 (m, 5H), 1.37 (d, J=7.2 Hz, 3H), 1.24-1.18 (m, 2H), 0.91-0.84 (m, 9H).

### Preparation T-6F7-E-6C4-CPT2 (DAR4 or DAR8):

Purified antibody T-6F7-E-6C4 is treated by under reducing conditions using Histidine to reduce the inter-chain disulfide bonds in the antibody. Then, 5.25 or 12.0 molar ratio of (II′)-2 in water for injection is conjugated via the reactive thiol group of the antibody by incubating for 1.5-2.0 hours at 20° C. The conjugation reaction is quenched by adding L-cysteine. The antibody-drug conjugates T-6F7-E-6C4-CPT2 (DAR4 or DAR8) are dialyzed using Pellicon 3 cassette with Ultracel. The conjugates are eluted from the Pellicon and can be concentrated and formulated using 20 mM Histidine pH6.0.

### In Vitro Killing Activities

Different concentrations of purified antibodies or ADCs were used to treat HCC827 cells, NCI-H292 cells, A431 cells, or Panc 02.03 cells cultured in a cell culture plate, and the killing activities were detected after 7 days of incubation using CellCounting-Lite 2.0 Kit Luminescent cell Viability Assay (Vazyme Biotech Co., Ltd., Cat #: DD1101-02). The results are shown in the table below.

TABLE 20

| | IC50 (nM) | | | |
|---|---|---|---|---|
| | | NCI- | | Panc |
| ADCs | HCC827 | H292 | A431 | 02.03 |
| T-6F7-E-6C4-CPT2 (DAR8) | 0.0598 | 0.6129 | 0.5048 | 14.12 |
| CPT2 | 1.6890 | 0.7275 | 1.3750 | 0.9806 |
| Sacituzumab govitecan | 0.3963 | — | — | 0.6639 |
| Cetuximab | 5.1250 | — | — | NA |

(“NA” means no in vitro killing activity; “—” means not tested)

The above results showed that T-6F7-E-6C4-CPT2 (DAR8) had good in vitro killing activity in HCC827 cells, NCI-H292 cells, A431 cells, and Panc 02.03 cells.

### Internalization of Anti-TROP2/EGFR Bispecific Antibody and ADCs

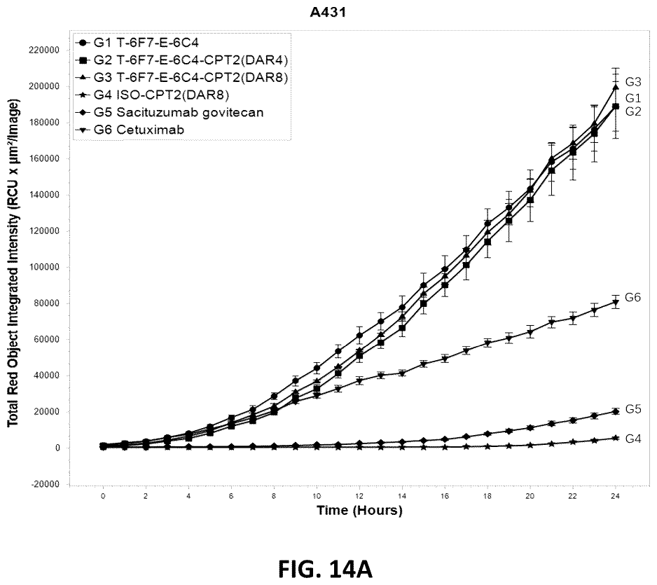

The anti-TROP2/EGFR bispecific antibody and ADCs (as shown in the table below) were used to treat A431 cells or NCI-H292 cells cultured in a cell culture plate, and the internalization activity was monitored over a 24-hour period after incubation using IncuCyte (Sartorius AG, IncuCyte® S3), with images captured every hour. The results are shown in FIGS. 14A-14B, which showed that the endocytosis activities of T-6F7-E-6C4-CPT2(DAR4), T-6F7-E-6C4-CPT2(DAR8) and T-6F7-E-6C4 were better than Sacituzumab govitecan and Cetuximab.

TABLE 21

| Group | Antibodies/ADCs |
|---|---|
| G1 | T-6F7-E-6C4 |
| G2 | T-6F7-E-6C4-CPT2(DAR4) |
| G3 | T-6F7-E-6C4-CPT2(DAR8) |
| G4 | ISO-CPT2(DAR8) |
| G5 | Sacituzumab govitecan |
| G6 | Cetuximab |

### Binding Activities of Anti-TROP2/EGFR Bispecific Antibody and ADCs

This experiment was performed to test the binding activities of the anti-TROP2/EGFR bispecific antibody and ADCs to tumor cell lines.

Specifically, A431 cells or human lung cancer HCC827 cells (ATCC, Cat #: CRL-2868) were transferred to a 96-well plate at a density of 2×10⁵ cells/well, respectively. Serially diluted anti-TROP2/EGFR bispecific antibody or ADCs (the highest concentration: 130 nM, diluted in a 2-fold series for 9 gradients) was added to the 96-well plate, and incubated at 4° C. for 25-30 minutes. Then, the cells were incubated with the secondary antibody Alexa Fluor® 647-conjugated AffiniPure F(ab′)₂ Fragment Goat Anti-Human IgG, Fcγ Fragment Specific (Jackson Immuno Research Laboratories, Inc., Cat #: 109-606-170) at 4° C. in the dark for 25-30 minutes before flow cytometry analysis. The results shown in the table below demonstrate that T-6F7-E-6C4-CPT2(DAR4), T-6F7-E-6C4-CPT2(DAR8), and T-6F7-E-6C4 can bind to A431 cells and HCC827 cells with high affinity.

TABLE 22

EC50 (nM)

| Antibodies/ADCs | A431 | HCC827 |
|---|---|---|
| T-6F7-E-6C4-CPT2(DAR4) | 1.914 | 1.582 |
| T-6F7-E-6C4-CPT2(DAR8) | 2.074 | 1.493 |
| T-6F7-E-6C4 | 2.504 | 1.987 |

### Example 11. Anti-Tumor Activity in Patient-Derived Breast Cancer Xenograft Model

B-NDG mice were engrafted in the right flank with breast cancer patient-derived tumor tissue fragments (2 mm×2 mm×2 mm). Immunofluorescence staining of patient-derived breast tumor fragments was performed and the images were analyzed via HALO 3.2 version. The results showed that EGFR-positive cells and TROP2-positive cells in the tumor fragments were 96.92% and 49.87%, respectively. When the tumors in the mice reached a volume of about 200-300 mm³, the mice were randomly placed into different groups based on the tumor volume. The mice were then injected with PBS or ADCs by i.v. administration. Details of the dosing schedule, route, and frequency are shown in the table below.

TABLE 23

| | | | | | | | Total |
|---|---|---|---|---|---|---|---|
| | No. of | | | | | | No. of |
| Group | mice | ADCs | Dosage | | Route | Frequency | ad [symbol] inistration |
| G1 | 5 | PBS | — | | i.v. | [symbol] | 1 |
| G2 | 5 | T-6F7-E-6C4-ADC | 3 | mg/kg | i.v. | [symbol] | 1 |
| G3 | 5 | T-6F7-E-6C4-CPT2(DAR4) | 3 | mg/kg | i.v. | [symbol] | 1 |
| G4 | 5 | T-6F7-E-6C4-CPT2(DAR4) | 6 | mg/kg | i.v. | [symbol] | 1 |
| G5 | 5 | T-6F7-E-6C4-CPT2(DAR4) | 10 | mg/kg | i.v. | [symbol] | 1 |
| G6 | 5 | T-6F7-E-6C4-CPT2(DAR8) | 3 | mg/kg | i.v. | [symbol] | 1 |
| G7 | 5 | T-6F7-E-6C4-CPT2(DAR8) | 6 | mg/kg | i.v. | [symbol] | 1 |
| G8 | 5 | T-6F7-E-6C4-CPT2(DAR8) | 10 | mg/kg | i.v. | [symbol] | 1 |

[symbol]

indicates data missing or illegible when filed

The table below summarizes the results for this experiment, including the tumor volumes on the day of grouping (Day 0), 17 days after grouping (Day 17) and 35 days after grouping (Day 35); TGI (%); the ratio of tumor-free mice on Day 35; and the statistical differences (P value) of tumor volume between the treatment and control groups.

TABLE 24

| | | | | Tumor- | TGI | P |
|---|---|---|---|---|---|---|
| | Tumor volume (mm³) | | | free | (%) | value |
| Group | Day 0 | Day 17 | Day 35 | (Day 35) | (Day 35) | (Day 35) |
| G1 | 205 ± 20 | 899 ± 132 | 1684 ± 345 | 0/5 | NA | NA |
| G2 | 204 ± 31 | 144 ± 41 | 629 ± 226 | 0/5 | 71.2 | 0.036 |
| G3 | 205 ± 29 | 152 ± 54 | 450 ± 193 | 0/5 | 83.4 | 0.014 |
| G4 | 205 ± 26 | 122 ± 24 | 370 ± 88 | 0/5 | 88.8 | 0.003 |
| G5 | 205 ± 33 | 43 ± 9 | 126 ± 45 | 0/5 | 105.3 | 0.001 |
| G6 | 205 ± 28 | 111 ± 36 | 332 ± 152 | 0/5 | 91.4 | 0.006 |
| G7 | 204 ± 30 | 29 ± 6 | 37 ± 15 | 2/5 | 111.3 | 0.001 |
| G8 | 203 ± 33 | 56 ± 25 | 48 ± 17 | 1/5 | 110.4 | 0.001 |

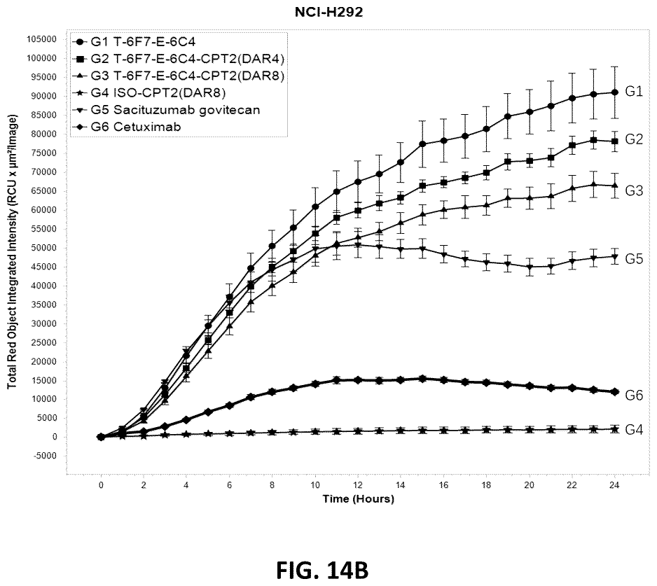

The tumor size in groups treated with PBS or ADCs are shown in FIG. 15. The results showed that T-6F7-E-6C4-CPT2 with DAR4 and DAR8 both exhibited good tumor inhibitory effects in a dose-dependent manner. In addition, T-6F7-E-6C4-CPT2 with DAR4 exhibited better tumor inhibitory effect than T-6F7-E-6C4-ADC at the dosage of 3 mg/kg.

### Example 12. Anti-Tumor Activity in Patient-Derived Pancreatic Cancer Xenograft Model

B-NDG mice were engrafted in the right flank with pancreatic cancer patient-derived tumor tissue fragments (2 mm×2 mm×2 mm). The immunofluorescence staining results showed that EGFR-positive cells and TROP2-positive cells in the pancreatic tumor fragments were 71.08% and 89.09%, respectively. When the tumors in the mice reached a volume of about 200-300 mm³, the mice were randomLy placed into different groups based on the tumor volume. The mice were then injected with PBS or ADCs by i.v. administration. Details are shown in the table below.

TABLE 25

| | | | | | | Total |
|---|---|---|---|---|---|---|
| | No. of | | | | | No. of |
| Group | mice | ADCs | Dosage | Route | Frequency | ad [symbol] inistration |
| G1 | 5 | PBS | — | i.v. | [symbol] | 1 |
| G2 | 5 | ISO-CPT2(DAR4) | 6 mg/kg | i.v. | [symbol] | 1 |
| G3 | 5 | T-6F7-E-6C4-CPT2(DAR4) | 1 mg/kg | i.v. | [symbol] | 1 |
| G4 | 5 | T-6F7-E-6C4-CPT2(DAR4) | 3 mg/kg | i.v. | [symbol] | 1 |
| G5 | 5 | T-6F7-E-6C4-CPT2(DAR4) | 6 mg/kg | i.v. | [symbol] | 1 |
| G6 | 5 | T-6F7-E-6C4-CPT2(DAR8) | 1 mg/kg | i.v. | [symbol] | 1 |
| G7 | 5 | T-6F7-E-6C4-CPT2(DAR8) | 3 mg/kg | i.v. | [symbol] | 1 |
| G8 | 5 | T-6F7-E-6C4-CPT2(DAR8) | 6 mg/kg | i.v. | [symbol] | 1 |

[symbol]

indicates data missing or illegible when filed

The table below summarizes the results for this experiment, including the tumor volumes on the day of grouping (Day 0), 14 days after grouping (Day 14) and 32 days after grouping (Day 32); TGI (%); and the statistical differences (P value) of tumor volume between the treatment and control groups.

TABLE 26

| | | | | TGI | P |
|---|---|---|---|---|---|
| | | | | (%) | value |
| | Tumor volume (mm³) | | | (Day | (Day |
| Group | Day 0 | Day 14 | Day 32 | 32) | 32) |
| G1 | 233 ± 14 | 730 ± 84 | 1676 ± 310 | NA | NA |
| G2 | 233 ± 19 | 454 ± 41 | 1315 ± 163 | 25.0 | 0.294 |
| G3 | 233 ± 14 | 606 ± 109 | 1545 ± 146 | 9.1 | 0.721 |
| G4 | 233 ± 22 | 281 ± 67 | 1066 ± 292 | 42.3 | 0.225 |
| G5 | 233 ± 23 | 181 ± 22 | 807 ± 137 | 60.3 | 0.024 |
| G6 | 233 ± 30 | 531 ± 71 | 1592 ± 235 | 5.9 | 0.833 |
| G7 | 233 ± 19 | 226 ± 42 | 1111 ± 128 | 39.2 | 0.093 |
| G8 | 233 ± 27 | 67 ± 26 | 378 ± 142 | 89.9 | 0.005 |

The results showed that T-6F7-E-6C4-CPT2 with DAR4 and DAR8 both exhibited tumor inhibitory effects in a dose-dependent manner.

### Example 13. Anti-Tumor Activity in SKOV-3 Xenograft Model

The ADCs were tested for their effects on tumor growth in vivo in a xenograft model of ovarian adenocarcinoma. Specifically, about 5×10⁶ SKOV-3 cells (ATCC, Cat #: HTB-77) were injected subcutaneously in B-NDG mice. When the tumors in the mice reached a volume of about 300 mm³, the mice were randomLy placed into different groups based on tumor volume. The mice were then injected with PBS or ADCs by intravenous (i.v.) administration. Details are shown in the table below.

TABLE 27

| | | | | | | | Total |
|---|---|---|---|---|---|---|---|
| | No. of | | | | | | No. of |
| Group | mice | ADCs | Dosage | | Route | Frequency | ad [symbol] inistration |
| G1 | 5 | PBS | — | | i.v. | [symbol] | 2 |
| G2 | 5 | T-6F7-E-6C4-CPT2(DAR4) | 3 | mg/kg | i.v. | [symbol] | 2 |
| G3 | 5 | T-6F7-E-6C4-CPT2(DAR4) | 6 | mg/kg | i.v. | [symbol] | 2 |
| G4 | 5 | T-6F7-E-6C4-CPT2(DAR4) | 10 | mg/kg | i.v. | [symbol] | 2 |
| G5 | 5 | T-6F7-E-6C4-CPT2(DAR8) | 3 | mg/kg | i.v. | [symbol] | 2 |
| G6 | 5 | T-6F7-E-6C4-CPT2(DAR8) | 6 | mg/kg | i.v. | [symbol] | 2 |
| G7 | 5 | T-6F7-E-6C4-CPT2(DAR8) | 10 | mg/kg | i.v. | [symbol] | 2 |

[symbol]

indicates data missing or illegible when filed

The table below summarizes the results for this experiment, including the tumor volumes on the day of grouping (Day 0), 17 days after grouping (Day 17) and 35 days after grouping (Day 35); TGI (%); and the statistical differences (P value) of tumor volume between the treatment and control groups.

TABLE 28

| | | | | TGI | P |
|---|---|---|---|---|---|
| | | | | (%) | value |
| | Tumor volume (mm³) | | | (Day | (Day |
| Group | Day 0 | Day 17 | Day 35 | 35) | 35) |
| G1 | 263 ± 17 | 1112 ± 79 | 2813 ± 243 | NA | NA |
| G2 | 263 ± 11 | 652 ± 59 | 1410 ± 208 | 55.0 | 0.002 |
| G3 | 263 ± 13 | 523 ± 38 | 919 ± 92 | 74.3 | 8.494E−05 |
| G4 | 263 ± 12 | 372 ± 21 | 686 ± 93 | 83.4 | 3.747E−05 |
| G5 | 263 ± 9 | 528 ± 18 | 858 ± 95 | 76.7 | 6.959E−05 |
| G6 | 264 ± 13 | 421 ± 49 | 749 ± 34 | 81.0 | 3.033E−05 |
| G7 | 263 ± 10 | 219 ± 24 | 247 ± 25 | 100.6 | 5.877E−06 |

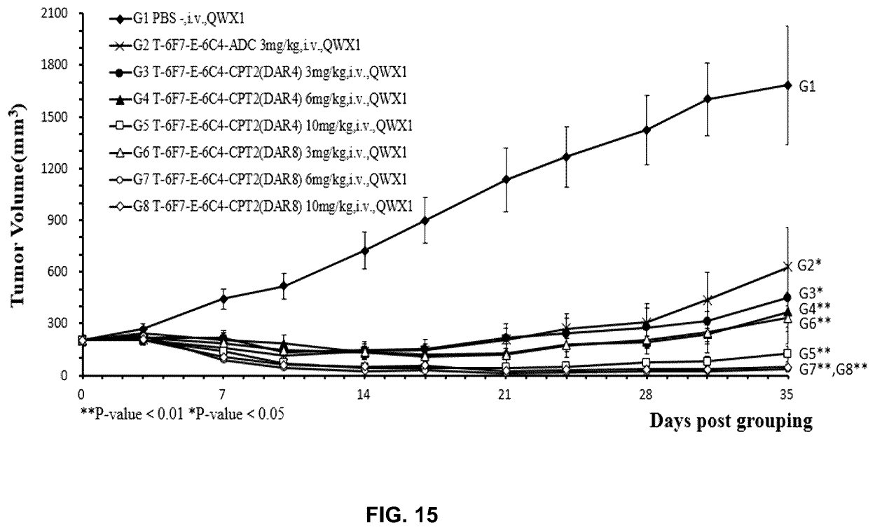

The tumor size in groups treated with PBS or ADCs are shown in FIG. 16. The results showed that T-6F7-E-6C4-CPT2 with DAR4 and DAR8 both exhibited tumor inhibitory effects in ovarian adenocarcinoma model in a dose-dependent manner, and T-6F7-E-6C4-CPT2(DAR8) exhibited better tumor inhibitory effects than T-6F7-E-6C4-CPT2(DAR4).

### Example 14. Anti-Tumor Activity in A431 Xenograft Model

The ADCs were tested for their effects on tumor growth in vivo in a xenograft model of epidermoid carcinoma. Specifically, about 1×10⁶ A431 cells were injected subcutaneously in B-NDG mice. When the tumors in the mice reached a volume of about 200 mm³, the mice were randomLy placed into different groups based on tumor volume. The mice were then injected with PBS, antibody, or ADCs by intravenous (i.v.) administration. Details are shown in the table below.

TABLE 29

| | | | | | | | Total |
|---|---|---|---|---|---|---|---|
| | No. of | Antibodies/ | | | | | No. of |
| Group | mice | ADCs | Dosage | | Route | Frequency | ad [symbol] inistration |
| G1 | 5 | PBS | — | | i.v. | [symbol] | 2 |
| G2 | 5 | ISO-CPT2(DAR4) | 10 | mg/kg | i.v. | [symbol] | 2 |
| G3 | 5 | ISO-CPT2(DAR8) | 10 | mg/kg | i.v. | [symbol] | 2 |
| G4 | 5 | Sacituzumab govitecan | 10 | mg/kg | i.v. | BI [symbol] | 4 |
| G5 | 5 | T-6F7-E-6C4-CPT2(DAR4) | 3 | mg/kg | i.v. | [symbol] | 2 |
| G6 | 5 | T-6F7-E-6C4-CPT2(DAR4) | 6 | mg/kg | i.v. | [symbol] | 2 |
| G7 | 5 | T-6F7-E-6C4-CPT2(DAR4) | 10 | mg/kg | i.v. | [symbol] | 2 |
| G8 | 5 | Cetuximab | 10 | mg/kg | i.v. | BI [symbol] | 4 |
| G9 | 5 | T-6F7-E-6C4-CPT2(DAR8) | 3 | mg/kg | i.v. | [symbol] | 2 |
| G10 | 5 | T-6F7-E-6C4-CPT2(DAR8) | 6 | mg/kg | i.v. | [symbol] | 2 |
| G11 | 5 | T-6F7-E-6C4-CPT2(DAR8) | 10 | mg/kg | i.v. | [symbol] | 2 |

[symbol]

indicates data missing or illegible when filed

The body weights were measured twice a week. During the experiment, body weights of mice in all groups increased and there was no significant difference in body weights among groups, indicating that the tested ADCs were well tolerated and not obviously toxic to the mice.

The table below summarizes the results for this experiment, including the tumor volumes on the day of grouping (Day 0), 17 days after grouping (Day 17) and 31 days after grouping (Day 31); TGI (%); and the statistical differences (P value) of tumor volume between the treatment and control groups.

TABLE 30

| | | | | TGI | P |
|---|---|---|---|---|---|
| | | | | (%) | value |
| | Tumor volume (mm³) | | | (Day | (Day |
| Group | Day 0 | Day 17 | Day 31 | 31) | 31) |
| G1 | 197 ± 10 | 1204 ± 66 | 2079 ± 191 | NA | NA |
| G2 | 197 ± 11 | 946 ± 123 | 1684 ± 258 | 21.0 | 0.253 |
| G3 | 196 ± 3 | 634 ± 69 | 1439 ± 168 | 34.0 | 0.036 |
| G4 | 197 ± 6 | 682 ± 71 | 1554 ± 144 | 27.9 | 0.059 |
| G5 | 197 ± 4 | 601 ± 54 | 1446 ± 175 | 33.6 | 0.040 |
| G6 | 197 ± 7 | 209 ± 23 | 657 ± 79 | 75.5 | 1.247E−04 |
| G7 | 196 ± 5 | 135 ± 13 | 189 ± 29 | 100.4 | 9.814E−06 |
| G8 | 197 ± 10 | 373 ± 23 | 806 ± 59 | 67.6 | 2.145E−04 |
| G9 | 196 ± 4 | 282 ± 31 | 1166 ± 139 | 48.5 | 0.005 |
| G10 | 197 ± 4 | 155 ± 17 | 277 ± 35 | 95.7 | 1.451E−05 |
| G11 | 197 ± 5 | 103 ± 6 | 67 ± 7 | 106.9 | 5.681E−06 |

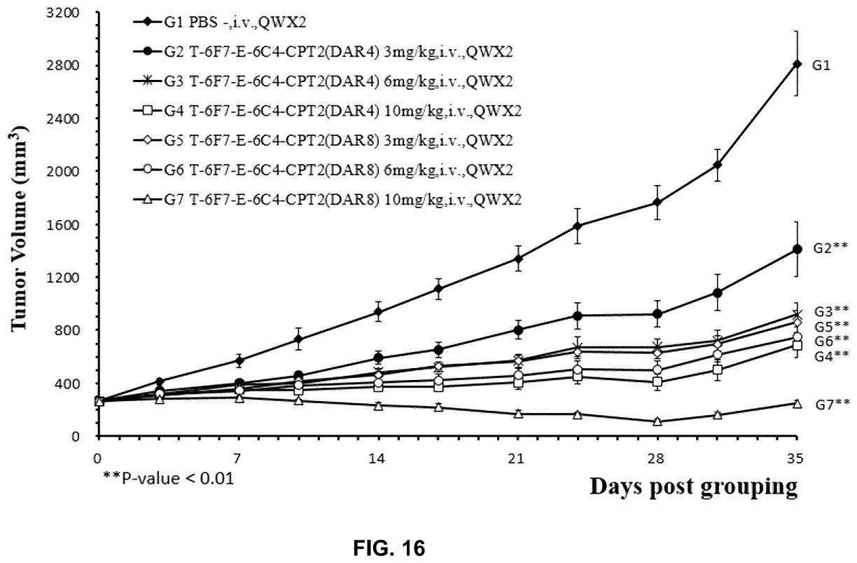

The tumor size in groups treated with PBS, antibody, or ADCs are shown in FIG. 17. The results showed that T-6F7-E-6C4-CPT2 with DAR4 and DAR8 both exhibited better tumor inhibitory effects than Sacituzumab govitecan or Cetuximab, in a dose-dependent manner. Further, the experiment was continued until 49 days after grouping (Day 49), and T-6F7-E-6C4-CPT2 with DAR4 and DAR8 of 6 mg/kg or 10 mg/kg still showed tumor inhibitory effects.

### Example 15. Anti-Tumor Activity in NCI-H292 Xenograft Model

The antibody or ADCs were tested for their effects on tumor growth in vivo in a xenograft model of lung cancer. Specifically, about 5×10⁶ NCI-H292 cells were injected subcutaneously in B-NDG mice. When the tumors in the mice reached a volume of about 300 mm³, the mice were randomLy placed into different groups based on tumor volume. The mice were then injected with PBS, antibody, or ADCs by intravenous (i.v.) administration. Details are shown in the table below.

TABLE 31

| | | | | | | | Total |
|---|---|---|---|---|---|---|---|
| | No. of | Antibodies/ | | | | | No. of |
| Group | mice | ADCs | Dosage | | Route | Frequency | ad [symbol] inistration |
| G1 | 5 | PBS | — | | i.v. | [symbol] | 1 |
| G2 | 5 | ISO-CPT2(DAR4) | 10 | mg/kg | i.v. | [symbol] | 1 |
| G3 | 5 | ISO-CPT2(DAR8) | 10 | mg/kg | i.v. | [symbol] | 1 |
| G4 | 5 | Sacituzumab govitecan | 10 | mg/kg | i.v. | BI [symbol] | 2 |
| G5 | 5 | T-6F7-E-6C4-CPT2(DAR4) | 3 | mg/kg | i.v. | [symbol] | 1 |
| G6 | 5 | T-6F7-E-6C4-CPT2(DAR4) | 6 | mg/kg | i.v. | [symbol] | 1 |
| G7 | 5 | T-6F7-E-6C4-CPT2(DAR4) | 10 | mg/kg | i.v. | [symbol] | 1 |
| G8 | 5 | Cetuximab | 10 | mg/kg | i.v. | BI [symbol] | 2 |
| G9 | 5 | T-6F7-E-6C4-CPT2(DAR8) | 3 | mg/kg | i.v. | [symbol] | 1 |
| G10 | 5 | T-6F7-E-6C4-CPT2(DAR8) | 6 | mg/kg | i.v. | [symbol] | 1 |
| G11 | 5 | T-6F7-E-6C4-CPT2(DAR8) | 10 | mg/kg | i.v. | [symbol] | 1 |

[symbol]

indicates data missing or illegible when filed

The table below summarizes the results for this experiment, including the tumor volumes on the day of grouping (Day 0), 21 days after grouping (Day 21) and at the end of the experiment (Day 39); the survival rate of the mice; TGI (%); and the statistical differences (P value) of tumor volume between the treatment and control groups.

TABLE 32

| | | | | TGI | P |
|---|---|---|---|---|---|
| | | | | (%) | value |
| | Tumor volume (mm³) | | | (Day | (Day |
| Group | Day 0 | Day 21 | Day 39 | 39) | 39) |
| G1 | 297 ± 7 | 1227 ± 99 | 2172 ± 243 | NA | NA |
| G2 | 297 ± 6 | 880 ± 68 | 1723 ± 237 | 23.9 | 0.222 |
| G3 | 297 ± 7 | 361 ± 38 | 1051 ± 93 | 59.8 | 0.003 |
| G4 | 297 ± 11 | 428 ± 51 | 893 ± 62 | 68.2 | 0.001 |
| G5 | 297 ± 9 | 692 ± 109 | 1231 ± 83 | 50.2 | 0.006 |
| G6 | 297 ± 9 | 177 ± 34 | 812 ± 54 | 72.5 | 0.001 |
| G7 | 297 ± 7 | 24 ± 4 | 351 ± 52 | 97.1 | 8.164E−05 |
| G8 | 297 ± 11 | 592 ± 24 | 1390 ± 99 | 41.7 | 0.018 |
| G9 | 297 ± 10 | 499 ± 40 | 1051 ± 34 | 59.8 | 0.002 |
| G10 | 297 ± 8 | 52 ± 3 | 417 ± 61 | 93.6 | 1.126E−04 |
| G11 | 297 ± 8 | 22 ± 6 | 254 ± 25 | 102.3 | 2.242E−04 |

The results showed that T-6F7-E-6C4-CPT2 with DAR4 and DAR8 both exhibited good tumor inhibitory effects in lung cancer model in a dose-dependent manner. In addition, T-6F7-E-6C4-CPT2 with DAR4 and DAR8 both exhibited good tumor inhibitory effects with higher TGI than that of Sacituzumab govitecan or Cetuximab at the dose level of 10 mg/kg.

### Example 16. Anti-Tumor Activity in Patient-Derived Xenograft Models

T-6F7-E-6C4-CPT2(DAR8) was tested for its effect on tumor growth in a head and neck squamous cell carcinoma model, an esophageal cancer model, a colorectal cancer model, or a gastric cancer model. Specifically, BALB/c nude mice were engrafted with patient-derived tumor tissue fragments (2 mm×2 mm×2 mm). When the tumors in the mice reached a volume of about 100-200 mm³, the mice were randomly placed into different groups based on tumor volumes (3 mice in each group). The mice were then injected with saline (G1, control) or 6 mg/kg T-6F7-E-6C4-CPT2(DAR8) (G2) (1 injection in total).

Immunohistochemistry (IHC) staining of different patient-derived tumor tissues was performed, and the table below show the histochemistry score (H-score) of EGFR or TROP2 expression level in the patient-derived tumor tissues. Table 33 below also summarizes the TGI (%) of different patient-derived xenograft models.

TABLE 33

| | | | Days | |
|---|---|---|---|---|
| | H-score | | after | TGI |
| PDX model | EGFR | TROP2 | grouping | (%) |
| Esophageal cancer PDX | 217.11 | 205.27 | Day 21 | 113.59 |
| Head and neck squamous cell | / | / | Day 27 | 117.34 |
| carcinoma PDX | | | | |
| Colorectal cancer PDX1 | 173.83 | 212.69 | Day 25 | 94.41 |
| Colorectal cancer PDX2 | 231.38 | 39.98 | Day 21 | 45.74 |
| Gastric cancer PDX1 | 109.65 | 34.06 | Day 27 | 111.93 |
| Gastric cancer PDX2 | 248.67 | 278.37 | Day 27 | 131.44 |

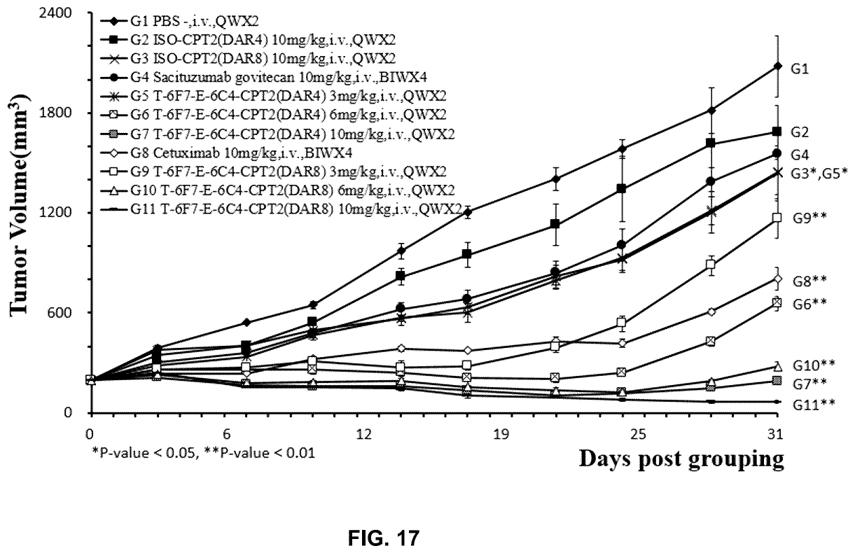

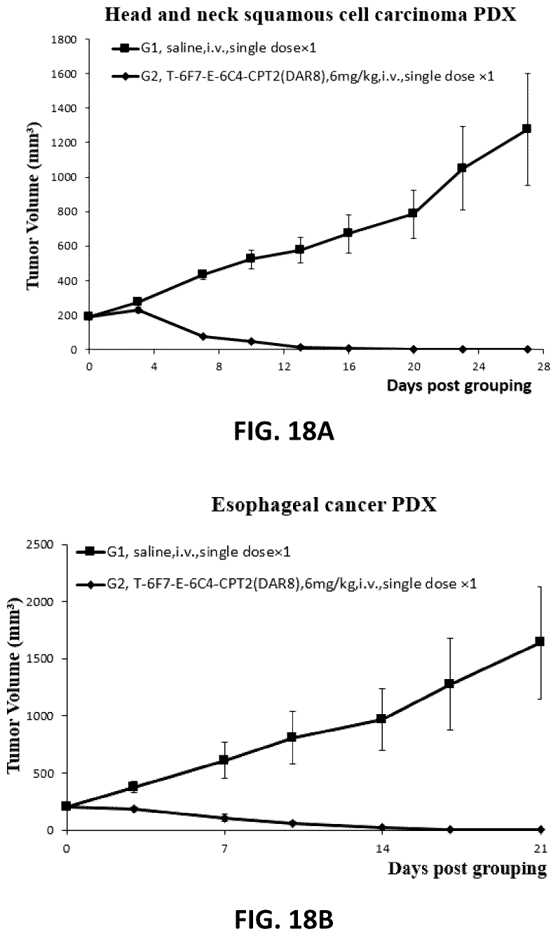

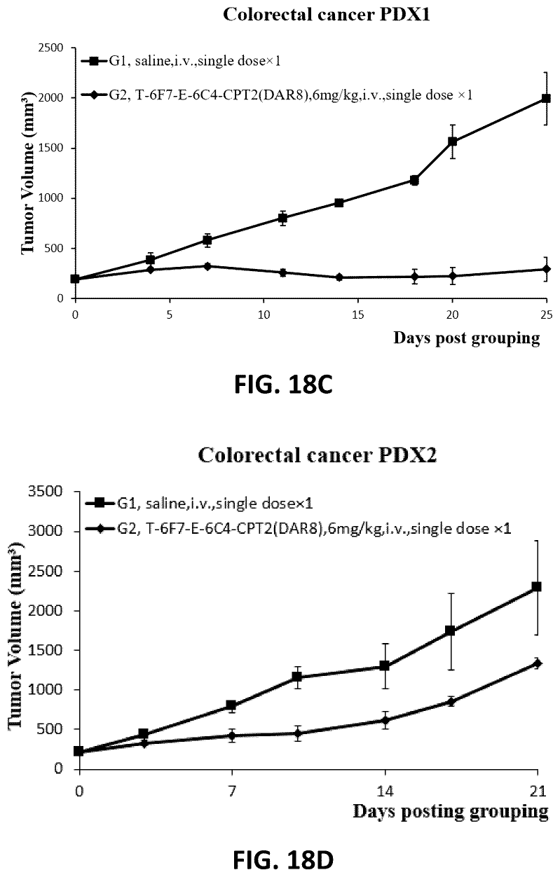

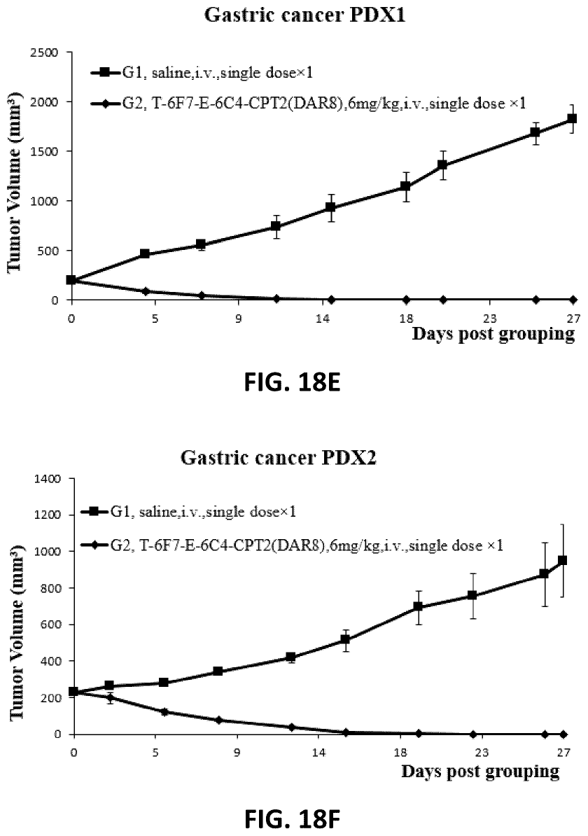

The tumor size in groups treated with saline or T-6F7-E-6C4-CPT2(DAR8) are shown in FIGS. 18A-18F, which showed that T-6F7-E-6C4-CPT2(DAR8) exhibited good tumor growth inhibition effects in head and neck squamous cell carcinoma, esophageal cancer, colorectal cancer, and gastric cancer.

### Example 17. Pharmacokinetic Profiles and Plasma Stability

The pharmacokinetic clearance rates of the anti-EGFR/TROP2 bispecific ADCs were determined in B-NDG mice. Specifically, about 1×10⁶ A431 cells were injected subcutaneously in B-NDG mice. When the tumors in the mice reached a volume of about 300 mm³, the mice were randomly placed into different groups based on tumor volume (3 mice per group) and then administered with PBS (G2), T-6F7-E-6C4-CPT2(DAR4) (G3-G10, 10 mg/kg), or T-6F7-E-6C4-CPT2(DAR8) (G11-G18, 10 mg/kg) by intravenous injection (one administration in total). G1 group was used as a blank control. Blood samples and tumor tissue samples of mice in group G3-G10 and G11-G18 were collected at 15 minutes, 2 hours, 6 hours, 1 day, 3 days, 5 days, 7 days, and 14 days after administration. Blood samples and tumor tissue samples of mice in G1 group were collected 1 hour before administration, while those of mice in G2 group were collected 14 days after administration. These collected samples were used to detect the total antibody levels in serum and tumor tissue by sandwich ELISA as well as free payload by MS (Mass Spectrometry).

The levels of total antibody were determined by sandwich ELISA. Briefly, Goat Anti-Human IgG (H+L) (Jackson ImmunoResearch Inc., Cat #: 109-005-088) was diluted to a final concentration of 2000 ng/mL, added to a 96-well plate (ELISA plate) at 100 μL/well, and then incubated overnight at 2-8° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of blocking buffer (2% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μL/well to each well of the plate, and incubated at 37° C. for 1 hour for determining the concentration of total antibody and payload CPT2. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. Luminescent signals of the plate were measured at 450 nm and 630 nm to calculate the concentrations. The absorbance value and corresponding concentration of the calibration sample prepared by each test product was used to create a standard curve with four parameters (i.e., T_{1/2}, Cₘₐₓ, AUC_{0-21 day}, and CL). The standard curve was used to calculate the antibody or ADC concentration of each serum sample. A drug concentration-time curve was created using the calculated sample concentration at each time point. Phoenix™ WinNolin 8.3 was used to calculate the pharmacokinetic parameters.

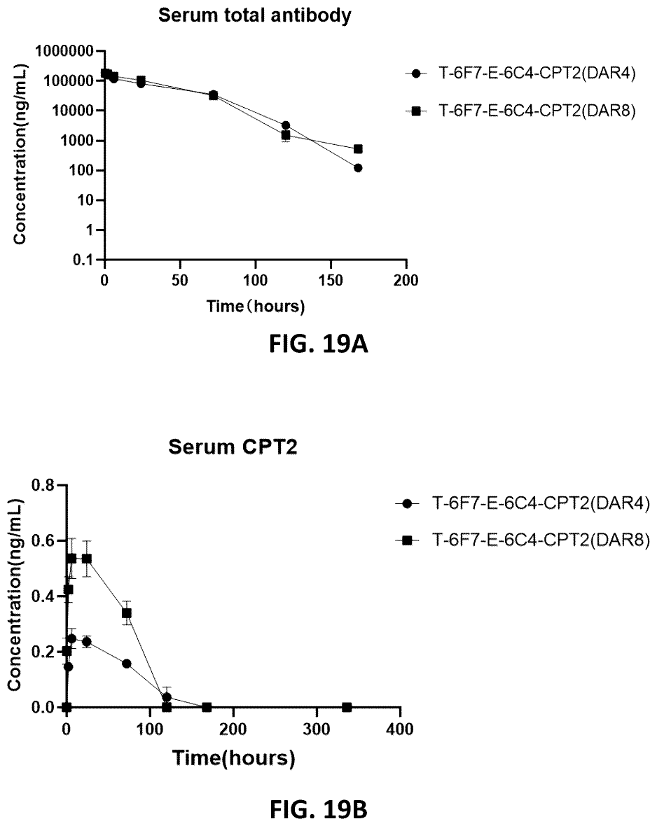

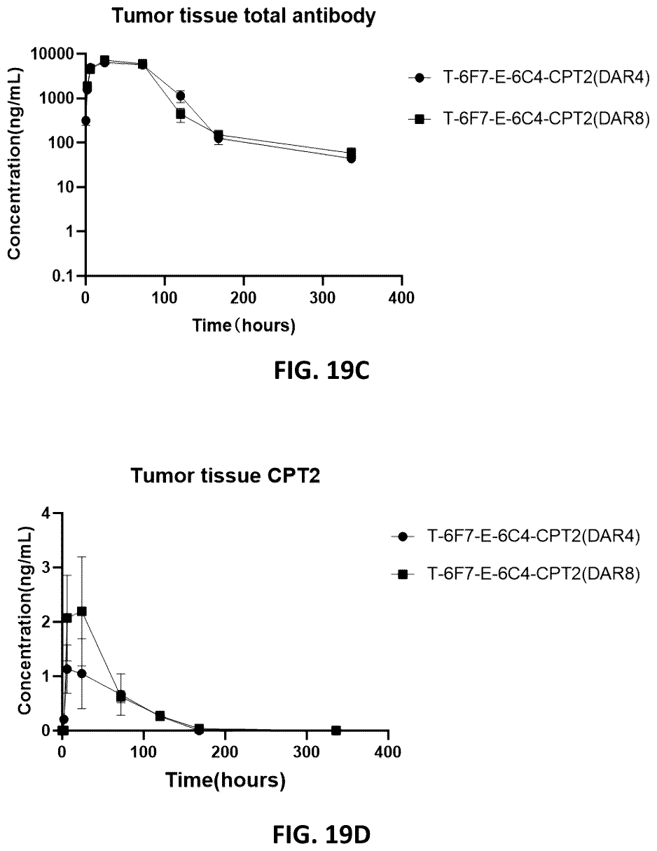

The results are shown in the table below and FIGS. 19A-19D, which showed that T-6F7-E-6C4-CPT2(DAR4) and T-6F7-E-6C4-CPT2(DAR8) exhibited expected PK behavior.

TABLE 34

| | Dose | | Cₘₐₓ | AUC₀₋ₗₐₛₜ | CL |
|---|---|---|---|---|---|
| ADCs | (mg/kg) | LBA | (ng/mL) | (hour*ng/mL) | (mL/hour/kg) |
| T-6F7- | 10 | Serum CPT2 | 0.25 | 21.33 | 2839.57 |
| E-6C4-CPT2 | | Serum antibody | 1206.14 | 42589.25 | 0 |
| (DAR4) | | Tumor tissue CPT2 | 3.96 | 357.1 | 237.47 |
| | | Tumor tissue antibody | 22267.93 | 2136100.75 | 4.66 |
| T-6F7- | 10 | Serum CPT2 | 0.54 | 33.18 | NA |
| E-6C4-CPT2 | | Serum antibody | 181302.67 | 7350021.62 | 1.36 |
| (DAR8) | | Tumor tissue CPT2 | 7.68 | 522.32 | 366.82 |
| | | Tumor tissue antibody | 25055.33 | 2168750.96 | 4.56 |

Cₘₐₓ: Max Concentration;
AUC₀₋ₗₐₛₜ: Area Under the Curve from Time Zero to Last Quantifiable Concentration
CL: Clearance

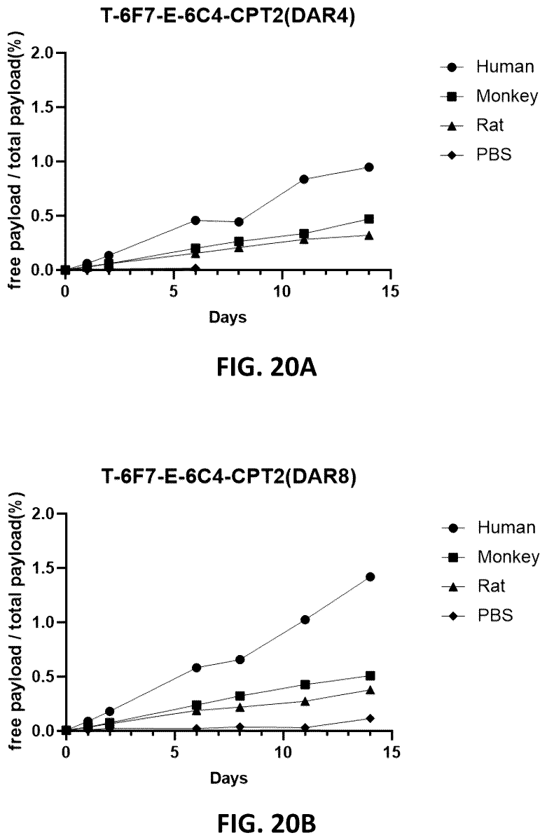

In another experiment, the plasma stability of T-6F7-E-6C4-CPT2(DAR4) and T-6F7-E-6C4-CPT2(DAR8) were determined in human plasma, monkey (Macaca fascicularis) plasma, and rat (SD rat) plasma. Specifically, T-6F7-E-6C4-CPT2(DAR4) or T-6F7-E-6C4-CPT2(DAR8) were added to human, monkey, or rat plasma, respectively, to a terminal concentration of 100 μg/mL. In the control group, the plasma was replaced by PBS with 0.5% BSA. The contents of free payload CPT2 and ADC were determined in 0 day, 1 day, 2 days, 6 days, 8 days, 11 days and 14 days after adding the ADCs, and the ratios of free CPT2 to the total ADC were calculated. The results are shown in FIGS. 20A-20B, which indicated that T-6F7-E-6C4-CPT2(DAR4) and T-6F7-E-6C4-CPT2(DAR8) were relatively stable in human, monkey and rat plasma, with a release rate of free CPT2 no more than 2.0% at the highest.

### Example 18. Toxicology Evaluation

In a preliminary experiment, to investigate the safety and toxicokinetics (TK) profile, T-6F7-E-6C4-CPT2(DAR8) was administered by i.v. injection to cynomolgus monkeys, three times with a 3-week interval (on Day 1, Day 22, and Day 43). The dose formulation is shown in the table below. Then, animals were sacrificed on Day 50 for gross and histopathological examination. Mortality/moribundity, general observations, body weights, food consumption, clinical pathology (hematology, coagulation, serum chemistry, and urinalysis), and gross lesions were evaluated. Blood samples were also collected for TK analysis and the primary TK parameters, e.g., Tmax, Cmax, and AUC (0-t) for payload, total antibody, and ADC were calculated. As a result, it was found that T-6F7-E-6C4-CPT2(DAR8) have a favorable safety profile.

TABLE 35

| | | Dose (mg/kg) | | |
|---|---|---|---|---|
| ADC | Group | Day 1 | Day 22 | Day 43 |
| T-6F7-E-6C4- | G1 | 5 | 20 | 20 |
| CPT2(DAR8) | G2 | 10 | 10 | 10 |
| | G3 | 30 | 30 | 30 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An anti-TROP2/EGFR antibody or antigen-binding fragment thereof, comprising:
a first antigen-binding domain that specifically binds to EGFR; and
a second antigen-binding domain that specifically binds to TROP2,
wherein the first antigen-binding domain comprises a first heavy chain variable region (VH1) and a first light chain variable region (VL1); and the second antigen-binding domain comprises a second heavy chain variable region (VH2) and a second light chain variable region (VL2).

2. The anti-TROP2/EGFR antibody or antigen-binding fragment thereof of claim 1, wherein:
the first heavy chain variable region (VH1) comprises VH1 CDR1, VH1 CDR2 and VH1 CDR3,
wherein the VH1 CDRs 1, 2 and 3 comprise a combination selected from the group consisting of:
(1) the VH1 CDRs 1, 2, 3 amino acid sequences are set forth in SEQ ID NOs: 7-9, respectively;
(2) the VH1 CDRs 1, 2, 3 amino acid sequences are set forth in SEQ ID NOs: 10-12, respectively;
(3) the VH1 CDRs 1, 2, 3 amino acid sequences are set forth in SEQ ID NOs: 16-18, respectively; and
(4) the VH1 CDRs 1, 2, 3 amino acid sequences are set forth in SEQ ID NOs: 19-21, respectively;
and
the first light chain variable region (VL1) comprises VL1 CDR1, VL1 CDR2 and VL1 CDR3,
wherein the VL1 CDRs 1, 2 and 3 comprise amino acid sequences set forth in SEQ ID NOs: 1-3, respectively.

3. The anti-TROP2/EGFR antibody or antigen-binding fragment thereof of claim 1, wherein:
the second heavy chain variable region (VH2) comprises VH2 CDR1, VH2 CDR2 and VH2 CDR3,
wherein the VH2 CDRs 1, 2 and 3 comprise a combination selected from the group consisting of:
(1) the VH2 CDRs 1, 2, 3 amino acid sequences are set forth in SEQ ID NOs: 4-6, respectively, and
(2) the VH2 CDRs 1, 2, 3 amino acid sequences are set forth in SEQ ID NOs: 13-15, respectively;
and
the second light chain variable region (VL2) comprises VL2 CDR1, VL2 CDR2 and VL2 CDR3,
wherein the VH2 CDRs 1, 2, and 3 amino acid sequences are set forth in SEQ ID NOs: 1-3, respectively.

4. The anti-TROP2/EGFR antibody or antigen-binding fragment thereof of claim 1, wherein:
(1) the VH1 CDRs 1, 2, 3 amino acid sequences are set forth in SEQ ID NOs: 7-9, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 4-6, respectively, and the VL2 CDRs 1, 2, 3 amino acid sequences are set forth in SEQ ID NOs: 1-3, respectively;
(2) the VH1 CDRs 1, 2, 3 amino acid sequences are set forth in SEQ ID NOs: 7-9, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 13-15, respectively, and the VL2 CDRs 1, 2, 3 amino acid sequences are set forth in SEQ ID NOs: 1-3, respectively;
(3) the VH1 CDRs 1, 2, 3 amino acid sequences are set forth in SEQ ID NOs: 16-18, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 4-6, respectively, and the VL2 CDRs 1, 2, 3 amino acid sequences are set forth in SEQ ID NOs: 1-3, respectively;
(4) the VH1 CDRs 1, 2, 3 amino acid sequences are set forth in SEQ ID NOs: 16-18, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 13-15, respectively, and the VL2 CDRs 1, 2, 3 amino acid sequences are set forth in SEQ ID NOs: 1-3, respectively;
(5) the VH1 CDRs 1, 2, 3 amino acid sequences are set forth in SEQ ID NOs: 10-12, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 4-6, respectively, and the VL2 CDRs 1, 2, 3 amino acid sequences are set forth in SEQ ID NOs: 1-3, respectively;
(6) the VH1 CDRs 1, 2, 3 amino acid sequences are set forth in SEQ ID NOs: 10-12, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 13-15, respectively, and the VL2 CDRs 1, 2, 3 amino acid sequences are set forth in SEQ ID NOs: 1-3, respectively;
(7) the VH1 CDRs 1, 2, 3 amino acid sequences are set forth in SEQ ID NOs: 19-21, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 4-6, respectively, and the VL2 CDRs 1, 2, 3 amino acid sequences are set forth in SEQ ID NOs: 1-3, respectively; or
(8) the VH1 CDRs 1, 2, 3 amino acid sequences are set forth in SEQ ID NOs: 19-21, respectively, and the VL1 CDRs 1, 2, 3 amino acid sequences are set forth in SEQ ID NOs: 1-3, respectively, and the VH2 CDRs 1, 2, 3 amino acid sequences are set forth in SEQ ID NOs: 13-15, respectively, and the VL2 CDRs 1, 2, 3 amino acid sequences are set forth in SEQ ID NOs: 1-3, respectively.

5. The anti-TROP2/EGFR antibody or antigen-binding fragment thereof of claim 1, wherein:
the first heavy chain variable region comprises a sequence that is at least 80%, 85%, 90%, 95%, 99%, or 100% identical to SEQ ID NO: 23, the first light chain variable region comprises a sequence that is at least 80%, 85%, 90%, 95%, 99%, or 100% identical to SEQ ID NO: 22, or
the first heavy chain variable region comprises a sequence that is at least 80%, 85%, 90%, 95%, 99%, or 100% identical to SEQ ID NO: 24, the first light chain variable region comprises a sequence that is at least 80%, 85%, 90%, 95%, 99%, or 100% identical to SEQ ID NO: 22; and
the second heavy chain variable region comprises a sequence that is at least 80%, 85%, 90%, 95%, 99%, or 100% identical to SEQ ID NO: 25, and the second light chain variable region comprises a sequence that is at least 80%, 85%, 90%, 95%, 99%, or 100% identical to SEQ ID NO: 22.

6. The anti-TROP2/EGFR antibody or antigen-binding fragment thereof of claim 1, wherein:
the VH1 comprises an amino acid sequence that is at least 90% identical to a selected VH sequence, and the VL1 comprises an amino acid sequence that is at least 90% identical to a selected VL sequence, wherein the selected VH sequence and the selected VL sequence are one of the following:
(1) the selected VH sequence is SEQ ID NO: 23, and the selected VL sequence is SEQ ID NO: 22; and
(2) the selected VH sequence is SEQ ID NO: 24, and the selected VL sequence is SEQ ID NO: 22;
and/or
the VH2 comprises an amino acid sequence that is at least 90% identical to SEQ ID NO: 25 and the VL2 comprises an amino acid sequence that is at least 90% identical to SEQ ID NO: 22.

7. The anti-TROP2/EGFR antibody or antigen-binding fragment thereof of claim 1, wherein:
the VH1 comprises VH CDR1, VH CDR2, and VH CDR3 that are identical to VH CDR1, VH CDR2, and VH CDR3 of a selected VH sequence; and the VL1 comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 23, and the selected VL sequence is SEQ ID NO: 22; and
(2) the selected VH sequence is SEQ ID NO: 24, and the selected VL sequence is SEQ ID NO: 22;
and/or
the VH2 comprises VH CDR1, VH CDR2, and VH CDR3 that are identical to VH CDR1, VH CDR2, and VH CDR3 of SEQ ID NO: 25; and the VL2 comprises VL CDR1, VL CDR2, and VL CDR3 that are identical to VL CDR1, VL CDR2, and VL CDR3 of SEQ ID NO: 22.

8. The anti-TROP2/EGFR antibody or antigen-binding fragment thereof of claim 1, wherein:
the first antigen-binding domain specifically binds to human or monkey EGFR; and/or the second antigen-binding domain specifically binds to human or monkey TROP2;
the first antigen-binding domain is human or humanized; and/or the second antigen-binding domain is human or humanized;
the antibody is a multi-specific antibody;
the first antigen-binding domain is a single-chain variable fragment (scFv); and/or the second antigen-binding domain is a scFv; and/or
the first light chain variable region and the second light chain variable region are identical.

9. An anti-TROP2/EGFR antibody or antigen-binding fragment thereof that cross-competes with the anti-TROP2/EGFR antibody or antigen-binding fragment thereof of claim 1.

10. A nucleic acid comprising a polynucleotide encoding the anti-TROP2/EGFR antibody or antigen-binding fragment thereof of claim 1.

11. An anti-TROP2/EGFR antibody-drug conjugate (ADC) comprising a therapeutic agent covalently bound to the anti-TROP2/EGFR antibody or antigen-binding fragment thereof of claim 1.

12. The ADC of claim 11, wherein the therapeutic agent is a cytotoxic or cytostatic agent.

13. The ADC of claim 11, wherein the therapeutic agent is selected from the group consisting of MMAE, MMAF,

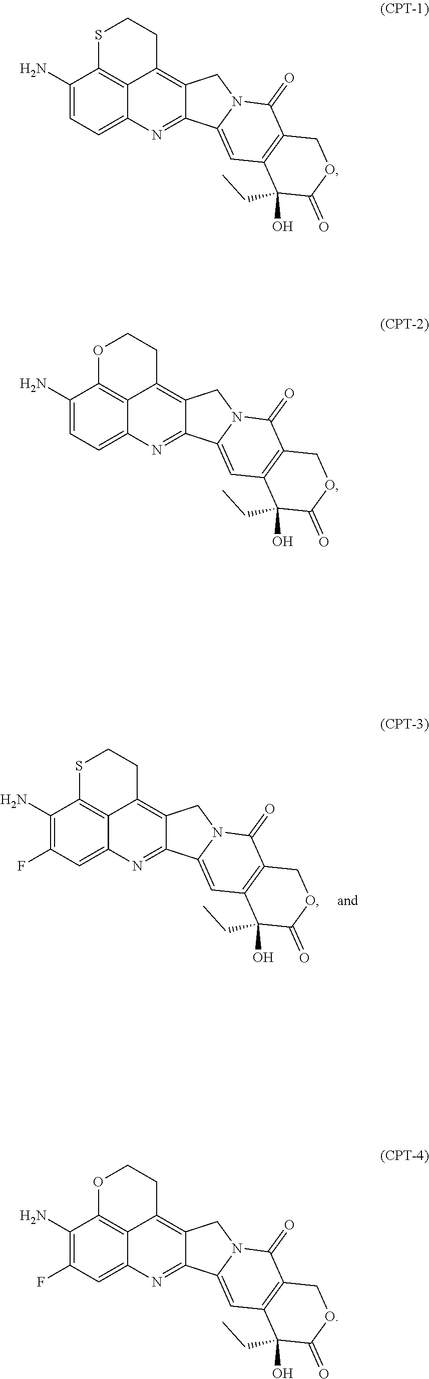 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

14. The ADC of claim 11, wherein the therapeutic agent is linked to the antibody or antigen-binding fragment thereof via a linker, wherein the linker has a structure of:

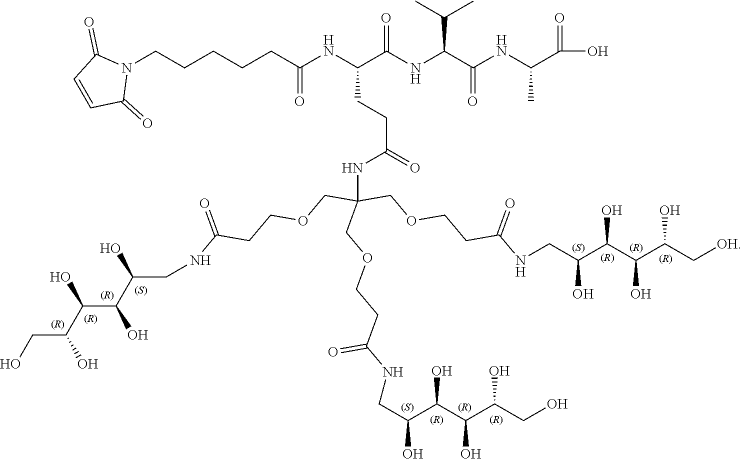

15. The ADC of claim 11, wherein the antibody-drug conjugate has a structure of: 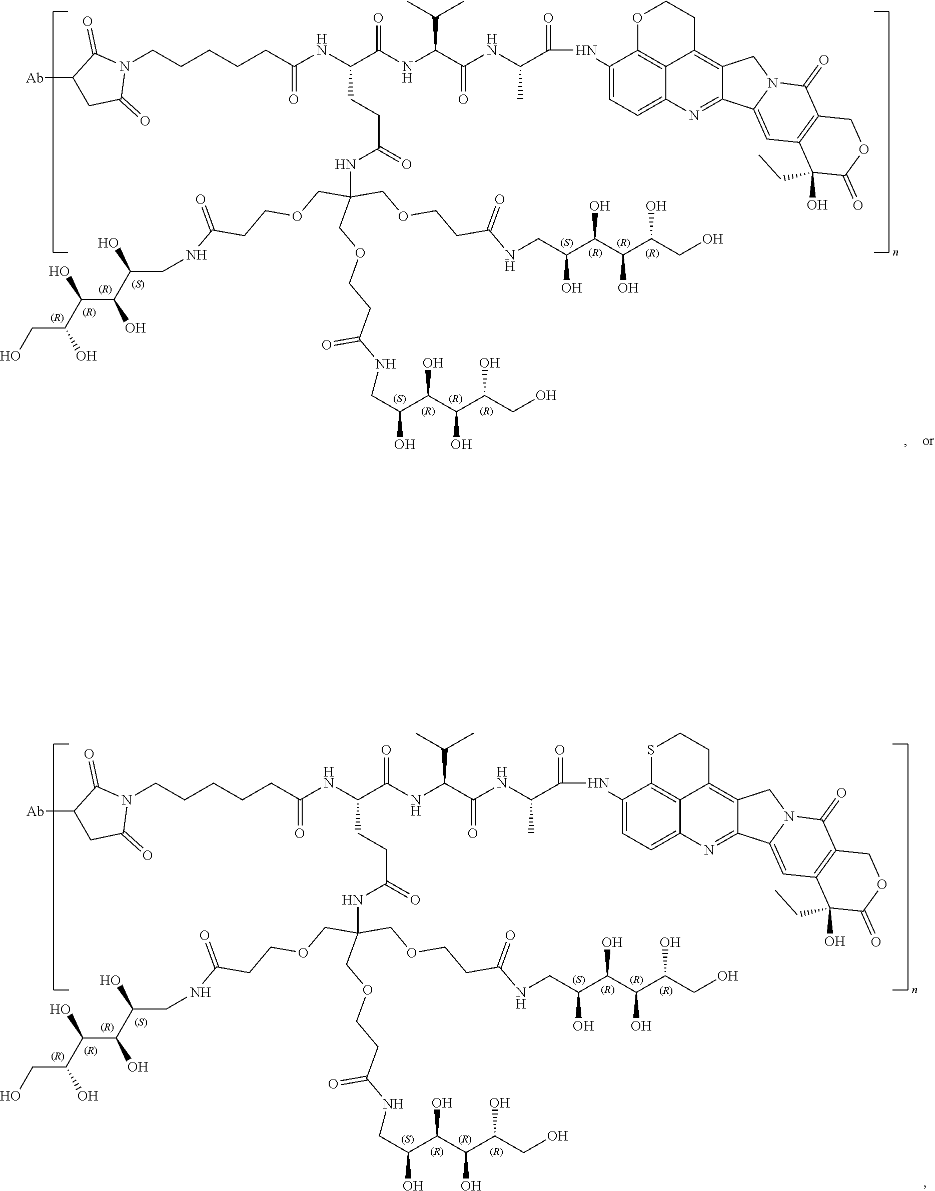
wherein n=1-8; wherein “Ab” represents the antibody or antigen-binding fragment thereof.

16. A method of treating a subject having cancer, the method comprising administering a therapeutically effective amount of a composition comprising the anti-TROP2/EGFR antibody or antigen-binding fragment thereof of claim 1 to the subject, wherein the subject has a cancer expressing EGFR and/or TROP2.

17. The method of claim 16, wherein the cancer is a solid tumor, lung cancer, gastric cancer, skin cancer, colorectal cancer, breast cancer, head and neck cancer, ovarian cancer, prostate cancer, thyroid cancer, pancreatic cancer, CNS cancer, liver cancer, nasopharynx cancer, brain cancer, colon cancer, bladder cancer, oral squamous cell carcinoma, cervical cancer, or esophageal cancer.

18. The method of claim 16, wherein the method further comprises administering an anti-PD1 antibody or a chemotherapy to the subject.

19. A pharmaceutical composition comprising a pharmaceutically acceptable carrier and the anti-TROP2/EGFR antibody or antigen-binding fragment thereof of claim 1.

20. The ADC of claim 11, wherein the drug-to-antibody ratio (DAR) is about 4 or 8.


SEQUENCE LISTING (36 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 7 aa, synthetic construct
SGYYYWG

SEQ ID NO: 5 - protein, 16 aa, synthetic construct
SIYYIGTTYYIPSLKS

SEQ ID NO: 6 - protein, 13 aa, synthetic construct
QPITVAGHDAFDI

SEQ ID NO: 7 - protein, 5 aa, synthetic construct
SYEMN

SEQ ID NO: 8 - protein, 17 aa, synthetic construct
YISSAGSTIYYADSVKG

SEQ ID NO: 9 - protein, 13 aa, synthetic construct
VWYSSGWSGCFAY

SEQ ID NO: 10 - protein, 7 aa, synthetic construct
SGDYYWS

SEQ ID NO: 11 - protein, 16 aa, synthetic construct
YIYYSGSTYYNPSLKS

SEQ ID NO: 12 - protein, 10 aa, synthetic construct
ERVYSSSLDY

SEQ ID NO: 13 - protein, 9 aa, synthetic construct
GGSIDSGYY

SEQ ID NO: 14 - protein, 5 aa, synthetic construct
YYIGT

SEQ ID NO: 15 - protein, 13 aa, synthetic construct
QPITVAGHDAFDI

SEQ ID NO: 16 - protein, 7 aa, synthetic construct
GFTFSSY

SEQ ID NO: 17 - protein, 6 aa, synthetic construct
SSAGST

SEQ ID NO: 18 - protein, 13 aa, synthetic construct
VWYSSGWSGCFAY

SEQ ID NO: 19 - protein, 9 aa, synthetic construct
GGSINSGDY

SEQ ID NO: 20 - protein, 5 aa, synthetic construct
YYSGS

SEQ ID NO: 21 - protein, 10 aa, synthetic construct
ERVYSSSLDY

SEQ ID NO: 22 - protein, 107 aa, synthetic construct [sequence omitted]

SEQ ID NO: 23 - protein, 122 aa, synthetic construct
QVQLVQSGGGLVQPGGSLRLSCAASGFTFSSYEMNWVRQAPGMGLEWVSYISSAGSTIYYADSVKGRFTISRDNAMNSLYLEMNSLRAEDTAVYYCVRVWYSSGWSGCFAYWGQGSLVTVSS

SEQ ID NO: 24 - protein, 120 aa, synthetic construct
QVQLQQWGPGLVKPSQTLSLTCTVSGGSINSGDYYWSWIRQPPGKGLESIGYIYYSGSTYYNPSLKSRVTISADTSKNQFSLKLTSVTAADTAVYYCARERVYSSSLDYWGQGTLVTVSS

SEQ ID NO: 25 - protein, 123 aa, synthetic construct
EVQLVQSGPGLVKPSETLSLTCTVSGGSIDSGYYYWGWIRQPPGKGLEWIGSIYYIGTTYYIPSLKSRVTISVDTSKNRFSLKLTSVTAADTAVYYCARQPITVAGHDAFDIWGQGTMVTVSS

SEQ ID NO: 26 - protein, 107 aa, synthetic construct [sequence omitted]

SEQ ID NO: 27 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 451 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
QVQLQESGPGLVKPSETLSLTCTVSGFSLSNYDVHWVRQAPGKGLEWLGVIWSGGNTDYNTPFTSRLTISVDTSKNQFSLKLSSVTAADTAVYYCARALDYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
EIVLTQSPDFQSVTPKEKVTITCRASQSIGTNIHWYQQKPDQSPKLLIKYASESISGIPSRFSGSGSGTDFTLTINSLEAEDAATYYCQQNNEWPTSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC